(19) United States
(12) Patent Application Publication
(10) Pub. No.: US 2024/0336612 A1
(43) Pub. Date: Oct. 10, 2024

(54) BRM Targeting Compounds And Associated Methods Of Use

(71) Applicant: Prelude Therapeutics Incorporated, Wilmington, DE (US)

(72) Inventors: Liang Lu, Hockessin, DE (US); Andrew Paul Combs, Kennett Square, PA (US)

(21) Appl. No.: 18/578,913

(22) PCT Filed: Jul. 12, 2022
(86) PCT No.: PCT/US2022/036821

§ 371 Date: Jan. 12, 2024

Related U.S. Application Data
(60) Provisional application No. 63/221,122, filed on Jul. 13, 2021.

(51) Int. Cl. C07D 471/20; A61K 31/5025

(52) U.S. Cl. CPC C07D 471/20; A61K 31/5025

(57) ABSTRACT

The present disclosure provides bifunctional compounds comprising a target protein binding moiety and a E3 ubiquitin ligase binding moiety, and associated methods of use.

### TECHNICAL FIELD OF THE INVENTION

The description provides bifunctional compounds comprising a target protein binding moiety and a E3 ubiquitin ligase binding moiety, and associated methods of use. The bifunctional compounds are useful as modulators of targeted ubiquitination, especially with respect to Switch/Sucrose Non-Fermentable (SWI/SNF)-Related, Matrix-Associated, Actin-Dependent Regulator of Chromatin, Subfamily A, Member 2 (SMARCA2) (i.e. BRAHMA or BRM), which are degraded and/or otherwise inhibited by bifunctional compounds according to the present disclosure.

### BACKGROUND OF THE INVENTION

The human SWItch/Sucrose Non-Fermentable (SWI/SNF) complexes are ATP-dependent chromatin remodelers. These large complexes play important roles in essential cellular processes, such as transcription, DNA repair and replication by regulating DNA accessibility.

Mutations in the genes encoding up to 20 canonical SWI/SNF subunits are observed in nearly 20% of all human cancers with the highest frequency of mutations observed in rhabdoid tumors, female cancers (including ovarian, uterine, cervical and endometrial), lung adenocarcinoma, gastric adenocarcinoma, melanoma, esophageal, and renal clear cell carcinoma.

SMARCA2 (BRM) and SMARCA4 (BRG1) are the subunits containing catalytic ATPase domains and they are essential for the function of SWI/SNF in perturbation of histone-DNA contacts, thereby providing access points to transcription factors and cognate DNA elements that facilitate gene activation and repression.

SMARCA2 and SMARCA4 shares a high degree of homology (up to 75%). SMARCA4 is frequently mutated in primary tumors (i.e., deleted or inactivated), particularly in lung cancer (12%), melanoma, liver cancer and pancreatic cancer. SMARCA2 is one of the top essential genes in SMARCA4-mutant (deleted) cancer cell line. This is because SMARCA4 deleted cancer cells exclusively rely on SMARCA2 ATPase activity for their chromatin remodeling activity for cellular functions such as cell proliferation, survival and growth. Thus, targeting SMARCA2 may be promising therapeutic approach in SMARCA4-related or deficient cancers (genetic synthetic lethality).

Previous studies have demonstrated the strong synthetic lethality using gene expression manipulation such as RNAi; downregulating SMARCA2 gene expression in SMARCA4 mutated cancer cells results in suppression of cancer cell proliferation. However, SMARCA2/4 bromodomain inhibitors (e.g. PFI-3) exhibit none to minor effects on cell proliferation inhibition [Vangamudi et al. Cancer Res 2015]. This phenotypic discrepancy between gene expression downregulation and small molecule-based approach lead us to investigating protein degradation bispecific molecules in SMARCA4 deficient cancers.

SMARCA2 is also reported to play roles in multiple myeloma expressing t(4;14) chromosomal translocation [Chooi et al. Cancer Res abstract 2018]. SMARCA2 interacts with NSD2 and regulates gene expression such as PRL3 and CCND1. SMARCA2 gene expression downregulation with shRNA reduces cell cycle S phase and suppresses cell proliferation of t(4;14) MM cells.

Therapeutic compounds that inhibit SMARCA2 and/or SMARCA4 are needed.

### SUMMARY OF THE INVENTION

The present disclosure is directed to compounds of Formula (I):

A′-L-B (I)

or a pharmaceutically acceptable salt or solvate thereof, wherein
A′ is a heterocycle that binds SMARCA2;
L is a bond or chemical moiety that links A′ and B;
B is a VHL binding moiety;
wherein A′ is defined as formula (II)

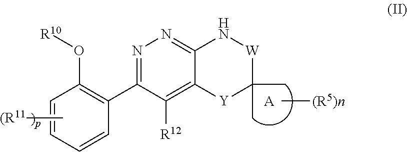

wherein
ring A is a carbocycle or a heterocycle;
W is bond or (CR¹R²)ₘ;
Y is a bond, (CR¹R²)ₘ, —O—(CR¹R²)ₘ, —S—(CR¹R²)ₘ, —S(O)x-(CR¹R²)ₘ, or —NR^{a}—(CR¹R²)ₘ;
m is 0, 1, 2 or 3;
n is 0, 1, 2, 3, or 4;
R¹⁰ is H, —C(O)R^{f}, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
p is 1, 2, 3 or 4;
each R¹¹ is independently H, D, halogen, —OH, —OR¹², —CN, —NO₂, —C₁-C₆alkyl, —C₂-C₆alkenyl, —C₂-C₆alkynyl;
each R¹² is independently H, D, halogen, C₁-C₄ alkoxide, C₁-C₄ alkyl, haloalkyl or CN;
each R¹, R², or R⁵ is independently H, D, halogen, —C₁-C₆ alkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl, C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)OR^{c}OR^{b}, —S(O)₂R^{b}, —S(O)₂NR^{c}R^{d}, or SiR^{b}₃; or R¹ and R² taken together with the carbon atom they are attached to form —C═O.
each R^{b}, is independently H, D, —C₁-C₆ alkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl;
or R^{c} and R^{d}, together with the atom to which they are both attached, form a monocyclic or multicyclic heterocycloalkyl, or a monocyclic or multicyclic heterocyclo-alkenyl group; and
wherein when L is a not a bond, L is a chemical moiety represented by the formula:

-(A)_{q}-,
wherein:
q is an integer from 1 to 14
each A is independently selected from the group consisting of CR^{1a}R^{1b}, O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR(CR^{1a}R^{1b})₁₋₄, NR^{1c}C(═NCN)NR^{1d}NR^{1c}C(═NCN), NR^{1c}C(═CNO₂)NR^{1d}, 3-11 membered cycloalkyl, optionally substituted with 0-6 R^{1a} and/or R^{1b} groups, 3-11 membered heterocyclyl optionally substituted with 0-6 R^{1a} and/or R^{1b} groups, aryl optionally substituted with 0-6 R^{1a} and/or R^{1b} groups, or heteroaryl optionally substituted with 0-6 R^{1a} and/or R^{1b} groups;
wherein R^{1a}, R^{1b}, R^{1c}, R^{1d} and R^{1e} are each independently, —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and where R^{1a} or R^{1b}, each independently may be optionally linked to other groups to form cycloalkyl and/or heterocyclyl moiety, optionally substituted with 0-4 R^{1e} groups;
or a pharmaceutically acceptable salt or solvate thereof.

### DETAILED DESCRIPTION OF THE INVENTION

Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (e.g., to at least one) of the grammatical object of the article unless the context clearly indicates otherwise. By way of example, “an element” means one element or more than one element.

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are co-administered in combination with at least one additional bioactive agent, especially including an anticancer agent. In particularly preferred aspects, the co-administration of compounds results in synergistic activity and/or therapy, including anticancer activity.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives, including prodrug and/or deuterated forms thereof where applicable, in context. Deuterated small molecules contemplated are those in which one or more of the hydrogen atoms contained in the drug molecule have been replaced by deuterium.

Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder.

The term “ubiquitin ligase” refers to a family of proteins that facilitate the transfer of ubiquitin to a specific substrate protein, targeting the substrate protein for degradation. For example, an E3 ubiquitin ligase protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead be altered in their cellular location or function, for example, via binding other proteins that have domains capable of binding ubiquitin. Further complicating matters, different lysines on ubiquitin can be targeted by an E3 to make chains. The most common lysine is Lys48 on the ubiquitin chain. This is the lysine used to make polyubiquitin, which is recognized by the proteasome.

As used herein, the term “alkyl”, by itself or as part of another substituent, means, unless otherwise stated, a straight or branched chain hydrocarbon radical having up to twelve carbon atoms. In some embodiments, the number of carbon atoms is designated (i.e., C₁-C₈ means one to eight carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. Alkyl groups may be optionally substituted as provided herein. In some embodiments, the alkyl group is a C₁-C₆ alkyl; in some embodiments, it is a C₁-C₄ alkyl.

When a range of carbon atoms is used herein, for example, C₁-C₆, all ranges, as well as individual numbers of carbon atoms are encompassed. For example, “C₁-C₃” includes C₁-C₃, C₁-C₂, C₂-C₃, C₁, C₂, and C₃.

The term “optionally substituted”, as used in combination with a substituent defined herein, means that the substituent may, but is not required to be, substituted with one or more suitable functional groups or other substituents as provided herein. For example, a substituent may be optionally substituted with one or more of: halo, cyano, C₁₋₆ alkyl, C₃₋₆ cycloalkyl, C₂₋₆ alkenyl, C₂₋₆ alkynyl, halo(C₁₋₆)alkyl, C₁₋₆ alkoxy, halo(C₁₋₆ alkoxy), C₁₋₆ alkylthio, C₁₋₆ alkylamino, NH₂, NH(C₁₋₆ alkyl), N(C₁₋₆ alkyl)₂, NH(C₁₋₆alkoxy), N(C₁₋₆ alkoxy)₂, —C(O)NHC₁₋₆ alkyl, —C(O)N(C₁₋₆ alkyl)₂, —C(O)NH₂, —C(O)C₁₋₆ alkyl, —C(O)₂C₁₋₆ alkyl, —NHCO(C₁₋₆ alkyl), —N(C₁₋₆ alkyl)CO(C₁₋₆ alkyl), —S(O)C₁₋₆ alkyl, —S(O)₂C₁₋₆ alkyl, oxo, 6-12 membered aryl, benzyl, pyridinyl, pyrazolyl, thiazolyl, isothiazolyl, or other 5 to 12 membered heteroaryl groups. In some embodiments, each of the above optional substituents are themselves optionally substituted by one or two groups.

The term “cycloalkyl” as used herein refers to a 3-12 membered cyclic alkyl group, and includes bridged and spirocycles (e.g., adamantine). Cycloalkyl groups may be fully saturated or partially unsaturated. The term “cycloalkyl” also includes multiple condensed ring systems (e.g., ring systems comprising 2, 3 or 4 rings) wherein a single cycloalkyl ring (as defined above) can be condensed with one or more groups selected from heterocycles, carbocycles, aryls, or heteroaryls to form the multiple condensed ring system. Such multiple condensed ring systems may be optionally substituted with one or more (e.g., 1, 2, 3 or 4) oxo groups on the carbocycle or heterocycle portions of the multiple condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a cycloalkyl) can be at any position of the cycloalkylic ring. Examples of cycloalkyl groups include cyclopropyl, cyclobutyl, cyclopentyl, cycloheptyl, cyclohexyl, cycloheptyl, cyclooctyl, indenyl, bicyclo[2.2.1]heptanyl, bicyclo[3.1.1]heptanyl, bicyclo[4.1.0]heptanyl, spiro[3.3]heptanyl, and spiro[3.4]octanyl. In some embodiments, the cycloalkyl group is a 3-7 membered cycloalkyl.

The term “akenyl” as used herein refers to C₂-C₁₂ alkyl group that contains at least one carbon-carbon double bond. In some embodiments, the alkenyl group is optionally substituted. In some embodiments, the alkenyl group is a C₂-C₆ alkenyl.

The term “akynyl” as used herein refers to C₂-C₁₂ alkyl group that contains at least one carbon-carbon triple bond. In some embodiments, the alkenyl group is optionally substituted. In some embodiments, the alkynyl group is a C₂-C₆ alkynyl.

The terms “alkoxy,” “alkylamino” and “alkylthio”, are used in their conventional sense, and refer to those alkyl groups attached to the remainder of the molecule via an oxygen atom (“oxy”), an amino group (“amino”) or thio group. The term “alkylamino” includes mono- di-alkylamino groups, the alkyl portions can be the same or different.

The terms “halo” or “halogen”, by itself or as part of another substituent, means a fluorine, chlorine, bromine, or iodine atom.

The term “heteroalkyl” refers to an alkyl group in which one or more carbon atom has been replaced by a heteroatom selected from S, O, P and N. Exemplary heteroalkyls include alkyl ethers, secondary and tertiary alkyl amines, alkyl amides, alkyl sulfides, and the like. The group may be a terminal group or a bridging group. As used herein reference to the normal chain when used in the context of a bridging group refers to the direct chain of atoms linking the two terminal positions of the bridging group.

The term “aryl” as used herein refers to a single, all carbon aromatic ring or a multiple condensed all carbon ring system wherein at least one of the rings is aromatic. For example, in certain embodiments, an aryl group has 6 to 12 carbon atoms. Aryl includes a phenyl radical. Aryl also includes multiple condensed ring systems (e.g., ring systems comprising 2, 3 or 4 rings) having about 9 to 12 carbon atoms in which at least one ring is aromatic and wherein the other rings may be aromatic or not aromatic. Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is to be understood that the point of attachment of a multiple condensed ring system, as defined above, can be at any position of the aromatic ring. Non-limiting examples of aryl groups include, but are not limited to, phenyl, indenyl, naphthyl, 1, 2, 3,4-tetrahydronaphthyl, and the like.

The term “heteroaryl” as used herein refers to a single aromatic ring that has at least one atom other than carbon in the ring, wherein the atoms are selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from heteroaryls (to form for example a naphthyridinyl such as 1,8-naphthyridinyl), heterocycles, (to form for example a 1, 2, 3, 4-tetrahydronaphthyridinyl such as 1,2,3,4-tetrahydro-1,8-naphthyridinyl), carbocycles (to form for example 5,6,7,8-tetrahydroquinolyl) and aryls (to form for example indazolyl) to form the multiple condensed ring system. Thus, a heteroaryl (a single aromatic ring or multiple condensed ring system) has about 1-20 carbon atoms and about 1-6 heteroatoms within the heteroaryl ring. A heteroaryl (a single aromatic ring or multiple condensed ring system) can also have about 5 to 12 or about 5 to 10 members within the heteroaryl ring. Multiple condensed ring systems may be optionally substituted with one or more (e.g., 1, 2, 3 or 4) oxo groups on the carbocycle or heterocycle portions of the condensed ring. The rings of a multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heteroaryl) can be at any position of the heteroaryl ring. It is also to be understood that the point of attachment for a heteroaryl or heteroaryl multiple condensed ring system can be at any suitable atom of the heteroaryl ring including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, triazolyl, 4,5,6,7-tetrahydro-TH-indazole and 3b,4,4a,5-tetrahydro-1H-cyclopropa[3,4]cyclo-penta[1,2-c]pyrazole. In one embodiment the term “heteroaryl” refers to a single aromatic ring containing at least one heteroatom. For example, the term includes 5-membered and 6-membered monocyclic aromatic rings that include one or more heteroatoms. Non-limiting examples of heteroaryl include but are not limited to pyridyl, furyl, thiazole, pyrimidine, oxazole, and thiadiazole.

The term “heterocyclyl” or “heterocycle” as used herein refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from heterocycles (to form for example a 1,8-decahydronapthyridinyl), carbocycles (to form for example a decahydroquinolyl) and aryls to form the multiple condensed ring system. Thus, a heterocycle (a single saturated or single partially unsaturated ring or multiple condensed ring system) has about 2-20 carbon atoms and 1-6 heteroatoms within the heterocycle ring. Such multiple condensed ring systems may be optionally substituted with one or more (e.g., 1, 2, 3 or 4) oxo groups on the carbocycle or heterocycle portions of the multiple condensed ring. The rings of the multiple condensed ring system can be connected to each other via fused, spiro and bridged bonds when allowed by valency requirements. It is to be understood that the individual rings of the multiple condensed ring system may be connected in any order relative to one another. Accordingly, a heterocycle (a single saturated or single partially unsaturated ring or multiple condensed ring system) has about 3-20 atoms including about 1-6 heteroatoms within the heterocycle ring system. It is also to be understood that the point of attachment of a multiple condensed ring system (as defined above for a heterocylyl) can be at any position of the heterocyclic ring. It is also to be understood that the point of attachment for a heterocycle or heterocycle multiple condensed ring system can be at any suitable atom of the heterocyclic ring including a carbon atom and a heteroatom (e.g., a nitrogen). In one embodiment the term heterocycle includes a C₂₋₂₀ heterocycle. In one embodiment the term heterocycle includes a C₂₋₇ heterocycle. In one embodiment the term heterocycle includes a C₂₋₅ heterocycle. In one embodiment the term heterocycle includes a C₂₋₄ heterocycle. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one N-methylpiperidine, imidazolidine, pyrazolidine, butyrolactam, valerolactam, imidazolidinone, hydantoin, dioxolane, phthalimide, 1,4-dioxane, thiomorpholine, thiomorpholine-S-oxide, thiomorpholine-S,S-oxide, pyran, 3-pyrroline, thiopyran, pyrone, tetrhydrothiophene, quinuclidine, tropane, 2-azaspiro[3.3]heptane, (1R,5S)-3-azabicyclo[3.2.1]octane, (1s,4s)-2-azabicyclo[2.2.2]octane, (1R,4R)-2-oxa-5-azabicyclo[2.2.2]octane and pyrrolidin-2-one. In one embodiment the term “heterocycle” refers to a monocyclic, saturated or partially unsaturated, 3-8 membered ring having at least one heteroatom. For example, the term includes a monocyclic, saturated or partially unsaturated, 4, 5, 6, or 7 membered ring having at least one heteroatom. Non-limiting examples of heterocycle include aziridine, azetidine, pyrrolidine, piperidine, piperidine, piperazine, oxirane, morpholine, and thiomorpholine. The term “9- or 10-membered heterobicycle” as used herein refers to a partially unsaturated or aromatic fused bicyclic ring system having at least one heteroatom. For example, the term 9- or 10-membered heterobicycle includes a bicyclic ring system having a benzo ring fused to a 5-membered or 6-membered saturated, partially unsaturated, or aromatic ring that contains one or more heteroatoms.

As used herein, the term “heteroatom” is meant to include oxygen (O), nitrogen (N), sulfur (S) and silicon (Si). The nitrogen and sulfur can be in an oxidized form when feasible.

As used herein, the term “chiral” refers to molecules which have the property of non-superimposability of the mirror image partner, while the term “achiral” refers to molecules which are superimposable on their mirror image partner.

As used herein, the term “stereoisomers” refers to compounds which have identical chemical constitution, but differ with regard to the arrangement of the atoms or groups in space, e.g., enantiomers, diastereomers, tautomers.

The term “patient” or “subject” is used throughout the specification to describe an animal, preferably a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The term “effective” is used to describe an amount of a compound, composition or component which, when used within the context of its intended use, effects an intended result. The term effective subsumes all other effective amount or effective concentration terms, which are otherwise described or used in the present application.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

“Treating” or “treatment” of any disease or disorder refers, in one embodiment, to ameliorating the disease or disorder (e.g., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

In an aspect, the present disclosure is directed to compounds of Formula (I):

A′-L-B (I)

or a pharmaceutically acceptable salt or solvate thereof, wherein
A′ is a heterocycle that binds SMARCA2;
L is a bond or chemical moiety that links A′ and B;
B is a VHL binding moiety;
wherein A′ is defined as formula (II)

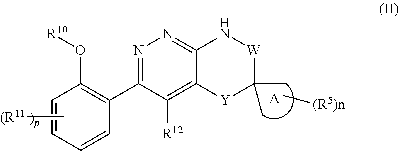

wherein
ring A is a carbocycle or a heterocycle;
W is bond or (CR¹R²)ₘ;
Y is a bond, (CR¹R²)ₘ, —O—(CR¹R²)ₘ, —S—(CR¹R²)ₘ, —S(O)x-(CR¹R²)ₘ, or —NR^{a}—(CR¹R²)ₘ;
m is 0, 1, 2 or 3;
n is 0, 1, 2, 3, or 4;
R¹⁰ is H, —C(O)R^{f}, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
p is 1, 2, 3 or 4;
each R¹¹ is independently H, D, halogen, —OH, —OR¹², —CN, —NO₂, —C₁-C₆alkyl, —C₂-C₆alkenyl, —C₂-C₆alkynyl;
each R¹² is independently H, D, halogen, C₁-C₄ alkoxide, C₁-C₄ alkyl, haloalkyl or CN;
each R¹, R², or R⁵ is independently H, D, halogen, —C₁-C₆ alkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl, C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)OR^{c}OR^{b}, —S(O)₂R^{b}, —S(O)₂NR^{c}R^{d}, or SiR^{b}₃; or R¹ and R² taken together with the carbon atom they are attached to form —C═O.
each R^{a} and R^{b}, is independently H, D, —C₁-C₆ alkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl;
or R^{c} and R^{d}, together with the atom to which they are both attached, form a monocyclic or multicyclic heterocycloalkyl, or a monocyclic or multicyclic heterocyclo-alkenyl group; and
wherein when L is a not a bond, L is a chemical moiety represented by the formula:

-(A)_{q}-,
wherein:
q is an integer from 1 to 14
each A is independently selected from the group consisting of CR^{1a}R^{1b}, O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR(CR^{1a}R^{1b})₁₋₄, NR^{1c}C(═NCN)NR^{1d}NR^{1c}C(═NCN), NR^{1c}C(═CNO₂)NR^{1d}, 3-11 membered cycloalkyl, optionally substituted with 0-6 R^{1a} and/or R^{1b} groups, 3-11 membered heteocyclyl optionally substituted with 0-6 R^{1a} and/or R^{1b} groups, aryl optionally substituted with 0-6 R^{1a} and/or R^{1b} groups, or heteroaryl optionally substituted with 0-6 R^{1a} and/or R^{1b} groups;
wherein R^{1a}, R^{1b}, R^{1c}, R^{1d} and R^{1e} are each independently, —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)₂, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and where R^{1a} or R^{1b}, each independently may be optionally linked to other groups to form cycloalkyl and/or heterocyclyl moiety, optionally substituted with 0-4 R^{1e} groups;
or a pharmaceutically acceptable salt or solvate thereof.

In some aspects, the compounds of Formula I include a protein targeting moiety (PTM) as shown in formula (II). In some aspects, the moiety of A′ defined as formula (II)

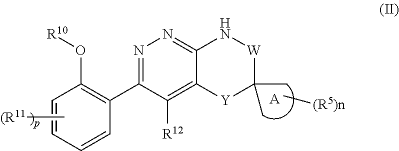

wherein
ring A is a carbocycle or a heterocycle;
W is bond or (CR¹R²)ₘ;
Y is a bond, (CR¹R²)ₘ, —O—(CR¹R²)ₘ, —S—(CR¹R²)ₘ, —S(O)x-(CR¹R²)ₘ, or —NR^{a}—(CR¹R²)ₘ;
m is 0, 1, 2 or 3;
n is 0, 1, 2, 3, or 4;
R¹⁰ is H, —C(O)R^{f}, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
p is 1, 2, 3 or 4;
each R¹¹ is independently H, D, halogen, —OH, —OR¹², —CN, —NO₂, —C₁-C₆alkyl, —C₂-C₆alkenyl, —C₂-C₆alkynyl;
each R¹² is independently H, D, halogen, C₁-C₄ alkoxide, C₁-C₄ alkyl, haloalkyl or CN;
each R¹, R², or R⁵ is independently H, D, halogen, —C₁-C₆ alkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl, C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)OR^{c}OR^{b}, —S(O)₂R^{b}, —S(O)₂NR^{c}R^{d}, or SiR^{b}₃; or R¹ and R² taken together with the carbon atom they are attached to form —C═O.
each R^{b}, is independently H, D, —C₁-C₆ alkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl;
or R^{c} and R^{d}, together with the atom to which they are both attached, form a monocyclic or multicyclic heterocycloalkyl, or a monocyclic or multicyclic heterocyclo-alkenyl group; and
wherein L is attached to A′ directly via ring A or is attached to A′ via an R¹, R² or R⁵ group.

According to the disclosure, ring A is a ring spirofused to the fused heterobicycle of formula II.

In some embodiments, ring A in Formula II is an optionally substituted 3-8 membered cycloalkyl ring or an optionally substituted 3-8 membered heterocyclic ring.

In some embodiments, ring A in Formula II is an optionally substituted 3-8 membered cycloalkyl ring.

In some embodiments, ring A in Formula II is an optionally substituted 3-8 membered cycloalkyl ring wherein the optional substituents are hydroxy, halogen, alkoxy, alkyl, haloalkyl, amino, alkylamino, or cyano.

In other embodiments, ring A in Formula II is an optionally substituted 3-8 membered heterocyclic ring.

In some embodiments, ring A in Formula II is an optionally substituted 3-8 membered heterocyclic ring wherein the optional substituents are hydroxy, halogen, alkoxy, alkyl, haloalkyl, amino, alkylamino, cyano.

In some embodiments, n in Formula II is 0-4. In some embodiments, n=0. In other embodiments, n=1. In other embodiments, n=2. In other embodiments, n=3. In other embodiments, n=4.

In some embodiments, m in Formula II is 0-3. In some embodiments, m=0. In other embodiments, m=1. In other embodiments, m=2. In other embodiments, m=3.

In some embodiments, p in Formula II is 1-4. In some embodiments, p=1. In other embodiments, p=2. In other embodiments, p=3. In other embodiments, p=4.

In some embodiments, W in Formula II is a bond or (CR¹R²)ₘ. In some embodiments, W is a bond. In some embodiments, W is (CR¹R²)ₘ. In some embodiments, W in Formula II is optionally substituted —CH₂—. In other embodiments, W in Formula II is —CH₂—.

In some embodiments, Y is Formula II is a bond, (CR¹R²)ₘ, —O—(CR¹R²)ₘ, —S—(CR¹R²)ₘ, —S(O)x-(CR¹R²)ₘ, or —NR^{a}—(CR¹R²)ₘ. In some embodiments, Y is a bond. In some embodiments, Y is (CR¹R²)ₘ. In some embodiments, Y is —O—(CR¹R²)ₘ. In some embodiments, Y is —S—(CR¹R²)ₘ. In some embodiments, Y is —S(O)x-(CR¹R²)ₘ. In some embodiments, Y is —NR^{a}—(CR¹R²)ₘ. In some embodiments, Y is —NR^{a}—. In some embodiments, Y is —NH.

In some embodiments, R¹⁰ in Formula II is H, —C(O)R^{f}, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl. In some embodiments, R¹⁰ is H. In some embodiments, R¹⁰ is —C(O)R^{f}. In some embodiments, R¹⁰ is —P(O)(OR^{g})₂.

In some embodiments, each R¹¹ in Formula II is independently H, D, halogen, —OH, —OR¹², —CN, —NO₂, —C₁-C₆alkyl, —C₂-C₆alkenyl, —C₂-C₆alkynyl. In some embodiments, R¹¹ is H. In some embodiments, R¹¹ is D. In some embodiments, R¹¹ is halogen. In some embodiments, R¹¹ is —OH. In some embodiments, R¹¹ is —OR¹². In some embodiments, R¹¹ is —CN. In some embodiments, R¹¹ is —NO₂. In some embodiments, R¹¹ is —C₁-C₆alkyl. In some embodiments, R¹¹ is —C₂-C₆alkenyl. In some embodiments, R¹¹ is —C₂-C₆alkynyl.

In some embodiments, each R¹² in Formula II is independently H, D, halogen, C₁-C₄ alkoxide, C₁-C₄ alkyl, haloalkyl or CN. In some embodiments, R¹² is H. In some embodiments, R¹² is D. In some embodiments, R¹² is halogen. In some embodiments, R¹² is —CN. In some embodiments, R¹² is C₁-C₄ alkoxide. In some embodiments, R¹² is C₁-C₄ alkyl. In some embodiments, R¹² is haloalkyl.

In some embodiments, each R¹ in Formula II is independently H, D, halogen, —C₁-C₆ alkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl, C(O)R^{b}, —C(O)OR^{c}, —C(O)NR^{c}R^{d}, —C(═NR)NR^{b}R^{c}, —C(═NOR)NR^{b}R^{c}, —C(═NCN)NR^{b}R^{c}, —P(OR^{c})₂, —P(O)OR^{c}OR^{b}, —S(O)₂R^{b}, —S(O)₂NR^{c}R^{d}, or SiR^{b}₃.

In some embodiments, R¹ is H. In some embodiments, R¹ is D. In some embodiments, R¹ is halogen. In some embodiments, R¹ is C₁-C₆ alkyl. In some embodiments, R¹ is C₁-C₆ alkyl. In some embodiments, R¹ is —C₂-C₆ alkenyl. In some embodiments, R¹ is —C₂-C₆ alkynyl. In some embodiments, R¹ is C₀-C₁alk-aryl. In some embodiments, R¹ is C₀-C₁alk-heteroaryl. In some embodiments, R¹ is heterocycloalkyl. In some embodiments, R¹ is heterocycloalkenyl. In some embodiments, R¹ is C(O)R^{b}. In some embodiments, R¹ is —C(O)OR^{c}. In some embodiments, R¹ is —C(O)NR^{c}R^{d}.

In some embodiments, each R² in Formula II is independently H, D, halogen, —C₁-C₆ alkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl, C(O)R^{b}, —C(O)OR^{c}, —C(O)NR^{c}R^{d}, —C(═NR)NR^{b}R^{c}, —C(═NOR)NR^{b}R^{c}, —C(═NCN)NR^{b}R^{c}, —P(OR^{c})₂, —P(O)OR^{c}OR^{b}, —S(O)₂R^{b}, —S(O)₂NR^{c}R^{d}, or SiR^{b}₃.

In some embodiments, R² is H. In some embodiments, R² is D. In some embodiments, R² is halogen. In some embodiments, R² is C₁-C₆ alkyl. In some embodiments, R² is C₁-C₆ alkyl. In some embodiments, R² is —C₂-C₆ alkenyl. In some embodiments, R² is —C₂-C₆ alkynyl. In some embodiments, R² is C₀-C₁alk-aryl. In some embodiments, R² is C₀-C₁alk-heteroaryl. In some embodiments, R² is heterocycloalkyl. In some embodiments, R² is heterocycloalkenyl. In some embodiments, R² is C(O)R^{b}. In some embodiments, R² is —C(O)OR. In some embodiments, R² is —C(O)NR^{c}R^{d}.

In some embodiments, R¹ and R² can be taken together with the carbon atom they are attached to form —C═O.

In some embodiments, each R⁵ in Formula II is independently H, D, halogen, —C₁-C₆ alkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl, C(O)R^{b}, —C(O)OR^{c}, —C(O)NR^{c}R^{d}, —C(═NR)NR^{b}R^{c}, —C(═NOR)NR^{b}R^{c}, —C(═NCN)NR^{b}R^{c}, —P(OR^{c})₂, —P(O)OR^{c}OR^{b}, —S(O)₂R^{b}, —S(O)₂NR^{c}R^{d}, or SiR^{b}₃.

In some embodiments, R⁵ is H. In some embodiments, R⁵ is D. In some embodiments, R⁵ is halogen. In some embodiments, R⁵ is C₁-C₆ alkyl. In some embodiments, R⁵ is C₁-C₆ alkyl. In some embodiments, R⁵ is —C₂-C₆ alkenyl. In some embodiments, R⁵ is —C₂-C₆ alkynyl. In some embodiments, R⁵ is C₀-C₁alk-aryl. In some embodiments, R⁵ is C₀-C₁alk-heteroaryl. In some embodiments, R⁵ is heterocycloalkyl. In some embodiments, R⁵ is heterocycloalkenyl. In some embodiments, R⁵ is C(O)R^{b}. In some embodiments, R⁵ is —C(O)OR^{C}. In some embodiments, R⁵ is —C(O)NR^{c}R^{d}.

In some embodiments, each R^{a} in Formula II is independently H, D, —C₁-C₆ alkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl.

In some embodiments, R^{a} is H. In some embodiments, R^{a} is D. In some embodiments, R^{a} is —C₁-C₆ alkyl. In some embodiments, R^{a} is —C₂-C₆ alkenyl. In some embodiments, R^{a} is —C₂-C₆ alkynyl. In some embodiments, R^{a} is C₀-C₁alk-aryl. In some embodiments, R^{a} is cycloalkyl. In some embodiments, R^{a} is cycloalkenyl. In some embodiments, R^{a} is C₀-C₁alk-heteroaryl. In some embodiments, R^{a} is heterocycloalkyl. In some embodiments, R^{a} is heterocycloalkenyl.

In some embodiments, each R^{b} in Formula II is independently H, D, —C₁-C₆ alkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl.

In some embodiments, R^{b} is H. In some embodiments, R^{b} is D. In some embodiments, R^{b} is —C₁-C₆ alkyl. In some embodiments, R^{b} is —C₂-C₆ alkenyl. In some embodiments, R^{b} is —C₂-C₆ alkynyl. In some embodiments, R^{b} is C₀-C₁alk-aryl. In some embodiments, R^{b} is cycloalkyl. In some embodiments, R^{b} is cycloalkenyl. In some embodiments, R^{b} is C₀-C₁alk-heteroaryl. In some embodiments, R^{b} is heterocycloalkyl. In some embodiments, R^{b} is heterocycloalkenyl.

In some embodiments, each R^{c} in Formula II is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl.

In some embodiments, R^{c} is H. In some embodiments, R^{c} is D. In some embodiments, R^{c} is —C₁-C₁₀ alkyl. In some embodiments, R^{c} is —C₂-C₆ alkenyl. In some embodiments, R^{c} is —C₂-C₆ alkynyl. In some embodiments, R^{c} is —OC₁-C₆alkyl. In some embodiments, R^{c} is —O— cycloalkyl. In some embodiments, R^{c} is aryl. In some embodiments, R^{c} is heteroaryl. In some embodiments, R^{c} is cycloalkyl. In some embodiments, R^{c} is cycloalkenyl. In some embodiments, R^{c} is heterocycloalkyl. In some embodiments, R^{c} is heterocycloalkenyl.

In some embodiments, each R^{d} in Formula II is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl.

In some embodiments, R^{d} is H. In some embodiments, R^{d} is D. In some embodiments, R^{d} is —C₁-C₁₀ alkyl. In some embodiments, R^{d} is —C₂-C₆ alkenyl. In some embodiments, R^{d} is —C₂-C₆ alkynyl. In some embodiments, R^{d} is —OC₁-C₆alkyl. In some embodiments, R^{d} is —O— cycloalkyl. In some embodiments, R^{d} is aryl. In some embodiments, R^{d} is heteroaryl. In some embodiments, R^{d} is cycloalkyl. In some embodiments, R^{d} is cycloalkenyl. In some embodiments, R^{d} is heterocycloalkyl.

In some embodiments, R^{c} and R^{d}, together with the atom to which they are both attached, form a monocyclic or multicyclic heterocycloalkyl, or a monocyclic or multicyclic heterocyclo-alkenyl group.

In some aspects, the disclosure is directed to compounds of formula III, formula IV, formula V, formula VI, or formula VII:

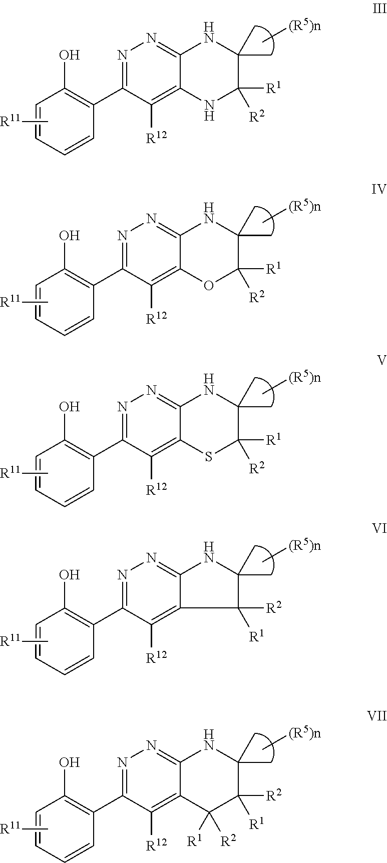

or a pharmaceutically acceptable salt or solvate thereof, wherein L is directly attached to ring A in any of the above formulae or wherein L is attached to ring A by an R⁵ group in any of the above formulae; or wherein L is attached to A′ by an R¹ or R² group in any of the above formulae.

In some embodiments, the compounds are compounds of formula III. In some embodiments, the compounds are compounds of formula IV. In some embodiments, the compounds are compounds of formula V. In some embodiments, the compounds are compounds of formula VI. In some embodiments, the compounds are compounds of formula VII.

In some aspects, the disclosure is directed to compounds of formula VIII, formula IX, formula X, formula XI, or formula XII.

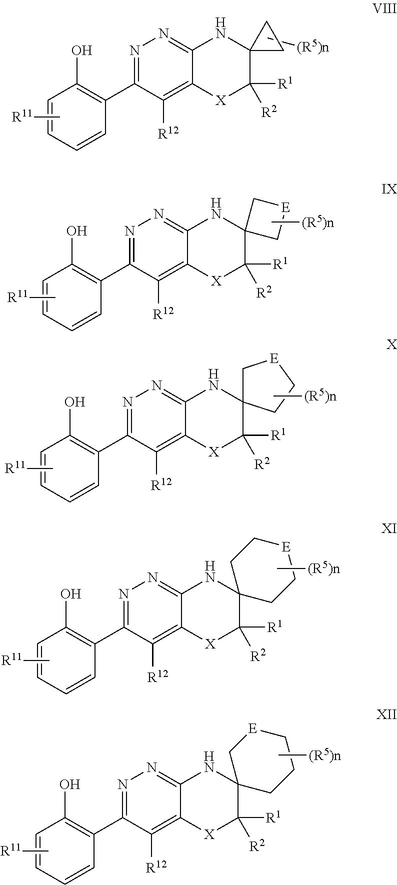

or a pharmaceutically acceptable salt or solvate thereof,
wherein E is CR¹R², O, C(O)NR^{c}R^{d}, N-L-B, C(R¹)(L-B), or NR^{b}; wherein when E is not N-L-B, C(R¹)(L-B), L is attached to ring A by an R¹, R², or R⁵ group included in E in any of the above formulae; or wherein L is attached to A′ by an R¹ or R² group in any of the above formulae; and wherein X is a bond, NR^{a}, O, S or CR¹R².

In some embodiments, the compounds are compounds of formula VIII

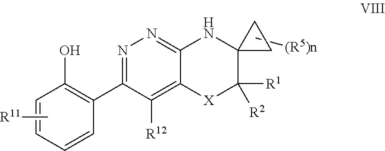

or a pharmaceutically acceptable salt or solvate thereof,
wherein L is directly attached to ring A or L is attached to ring A by an R⁵ group in the above formula; or wherein L is attached to A′ by an R¹ or R² group in the above formula and wherein X is a bond, NR^{a}, O, S or CR¹R².

In some embodiments, X in formula VIII is a bond. In some embodiments, X in formula VIII is NR^{a}. In some embodiments, X in formula VIII is O. In some embodiments, X in formula VIII is S. In some embodiments, X in formula VIII is CR¹R².

In some embodiments, L in formula VIII is attached to A′ by an R¹ group. In some embodiments, L in formula VIII is attached to A′ by an R² group. In some embodiments, L in formula VIII is attached to ring A by an R⁵ group. In some embodiments, L in formula VIII is directly attached to ring A.

In some embodiments, the compounds are compounds of formula IX

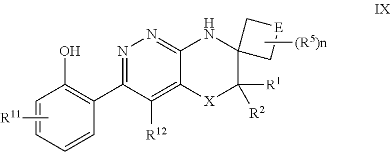

or a pharmaceutically acceptable salt or solvate thereof;
wherein E is CR¹R², O, C(O)NR^{c}R^{d}, N-L-B, C(R¹)(L-B), or NR^{b}; wherein when E is not N-L-B, C(R¹)(L-B), L is attached to ring A by an R¹, R² or R⁵ group included in E in the above formula; or wherein L is attached to A′ by an R¹ or R² group in the above formula; and wherein X is a bond, NR^{a}, O, S or CR¹R².

In some embodiments, E in formula IX is CR¹R². In some embodiments, E in formula IX is O. In some embodiments, E in formula IX is C(O)NR^{C}R^{d}. In some embodiments, E in formula IX is N-L-B. In some embodiments, E in formula IX is C(R¹)(L-B). In some embodiments, E in formula IX is NR^{b}.

In some embodiments, when E in formula IX is not N-L-B, C(R¹)(L-B), L in formula IX is attached to ring A by an R⁵ group. In some embodiments, when E in formula IX is not N-L-B, C(R¹)(L-B), L in formula IX is attached to ring A by an R¹ group included in E. In some embodiments, when E in formula IX is not N-L-B, C(R¹)(L-B), L in formula IX is attached to ring A by an R² group included in E. In some embodiments, when E in formula IX is not N-L-B, C(R¹)(L-B), L in formula IX is attached to A′ by an R¹ group. In some embodiments, when E in formula IX is not N-L-B, C(R¹)(L-B), L in formula IX is attached to A′ by an R² group.

In some embodiments, X in formula IX is a bond. In some embodiments, X in formula IX is NR^{a}. In some embodiments, X in formula IX is O. In some embodiments, X in formula IX is S. In some embodiments, X in formula IX is CR¹R².

In some embodiments, the compounds are compounds of formula X

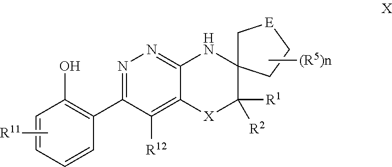

or a pharmaceutically acceptable salt or solvate thereof,
wherein E is CR¹R², O, C(O)NR^{c}R^{d}, N-L-B, C(R¹)(L-B), or NR^{b}; wherein when E is not N-L-B, C(R¹)(L-B), L is attached to ring A by an R¹, R² or R⁵ group included in E in the above formula; or wherein L is attached to A′ by an R¹ or R² group in the above formula; and wherein X is a bond, NR^{a}, O, S or CR¹R².

In some embodiments, E in formula X is CR¹R². In some embodiments, E in formula X is O. In some embodiments, E in formula X is C(O)NR^{c}R^{d}. In some embodiments, E in formula X is N-L-B. In some embodiments, E in formula X is C(R¹)(L-B). In some embodiments, E in formula X is NR^{b}.

In some embodiments, when E in formula X is not N-L-B, C(R¹)(L-B), L in formula X is attached to ring A by an R⁵ group. In some embodiments, when E in formula X is not N-L-B, C(R¹)(L-B), L in formula X is attached to ring A by an R¹ group included in E. In some embodiments, when E in formula X is not N-L-B, C(R¹)(L-B), L in formula X is attached to ring A by an R² group included in E. In some embodiments, when E in formula X is not N-L-B, C(R¹)(L-B), L in formula X is attached to A′ by an R¹ group. In some embodiments, when E in formula X is not N-L-B, C(R¹)(L-B), L in formula X is attached to A′ by an R² group.

In some embodiments, X in formula X is a bond. In some embodiments, X in formula X is NR^{a}. In some embodiments, X in formula X is O. In some embodiments, X in formula X is S. In some embodiments, X in formula X is CR¹R².

In some embodiments, the compounds are compounds of formula XI

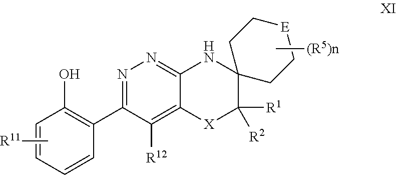

or a pharmaceutically acceptable salt or solvate thereof;
wherein E is CR¹R², O, C(O)NR^{c}R^{d}, N-L-B, C(R¹)(L-B), or NR^{b}; wherein when E is not N-L-B, C(R¹)(L-B), L is attached to ring A by an R¹, R² or R⁵ group not included in E in the above formula; or wherein L is attached to A′ by an R¹ or R² group included in E in the above formula; and wherein X is a bond, NR^{a}, O, S or CR¹R².

In some embodiments, E in formula XI is CR¹R². In some embodiments, E in formula XI is O. In some embodiments, E in formula XI is C(O)NR^{c}R^{d}. In some embodiments, E in formula IX is N-L-B. In some embodiments, E in formula XI is C(R¹)(L-B). In some embodiments, E in formula XI is NR^{b}.

In some embodiments, when E in formula XI is not N-L-B, C(R¹)(L-B), L in formula XI is attached to ring A by an R⁵ group. In some embodiments, when E in formula XI is not N-L-B, C(R¹)(L-B), L in formula XI is attached to ring A by an R¹ group included in E. In some embodiments, when E in formula XI is not N-L-B, C(R¹)(L-B), L in formula XI is attached to ring A by an R² group included in E. In some embodiments, when E in formula XI is not N-L-B, C(R¹)(L-B), L in formula XI is attached to A′ by an R¹ group. In some embodiments, when E in formula XI is not N-L-B, C(R¹)(L-B), L in formula XI is attached to A′ by an R² group.

In some embodiments, X in formula XI is a bond. In some embodiments, X in formula XI is NR^{a}. In some embodiments, X in formula XI is O. In some embodiments, X in formula XI is S. In some embodiments, X in formula XI is CR¹R².

In some embodiments, the compounds are compounds of formula XII

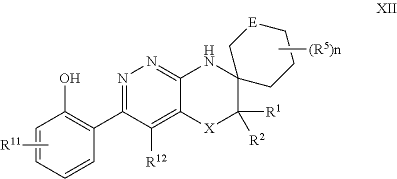

or a pharmaceutically acceptable salt or solvate thereof;
wherein E is CR¹R², O, C(O)NR^{c}R^{d}, N-L-B, C(R¹)(L-B), or NR^{b}; wherein when E is not N-L-B, C(R¹)(L-B), L is attached to ring A by an R¹, R² or R⁵ group included in E in any of the above formulae; or wherein L is attached to A′ by an R¹ or R² group in the above formula and wherein X is a bond, NR^{a}, O, S or CR¹R².

In some embodiments, E in formula XII is CR¹R². In some embodiments, E in formula XII is O. In some embodiments, E in formula XII is C(O)NR^{C}R^{d}. In some embodiments, E in formula IX is N-L-B. In some embodiments, E in formula XII is C(R¹)(L-B). In some embodiments, E in formula XII is NR^{b}.

In some embodiments, when E in formula XII is not N-L-B, C(R¹)(L-B), L in formula XII is attached to ring A by an R⁵ group. In some embodiments, when E in formula XII is not N-L-B, C(R¹)(L-B), L in formula XII is attached to ring A by an R¹ group included in E. In some embodiments, when E in formula XII is not N-L-B, C(R¹)(L-B), L in formula XII is attached to ring A by an R² group included in E. In some embodiments, when E in formula XII is not N-L-B, C(R¹)(L-B), L in formula XII is attached to A′ by an R¹ group. In some embodiments, when E in formula XII is not N-L-B, C(R¹)(L-B), L in formula XII is attached to A′ by an R² group.

In some embodiments, X in formula XII is a bond. In some embodiments, X in formula XII is NR^{a}. In some embodiments, X in formula XII is O. In some embodiments, X in formula XII is S. In some embodiments, X in formula XII is CR¹R².

In some aspects, the disclosure is directed to compounds of formula XIII, formula XIV, formula XV, formula XVI, formula XVII, formula XVIII, formula XIX, formula XX, formula XXI, or formula XXII.

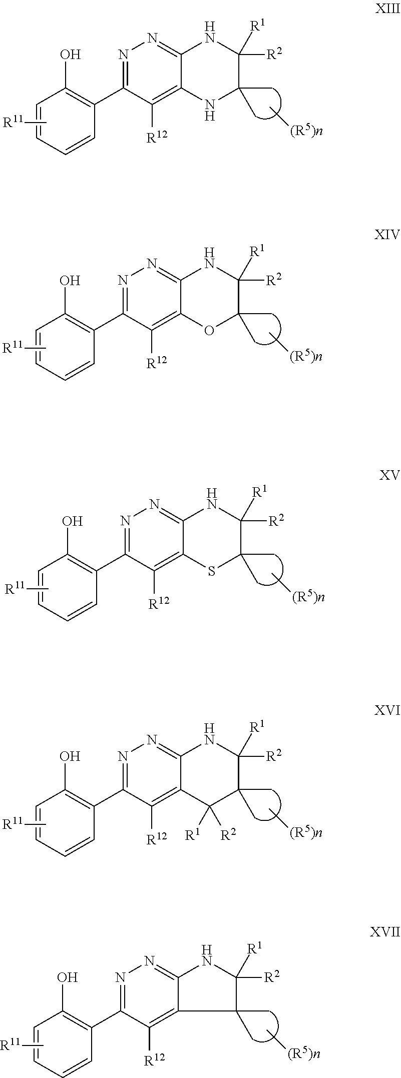

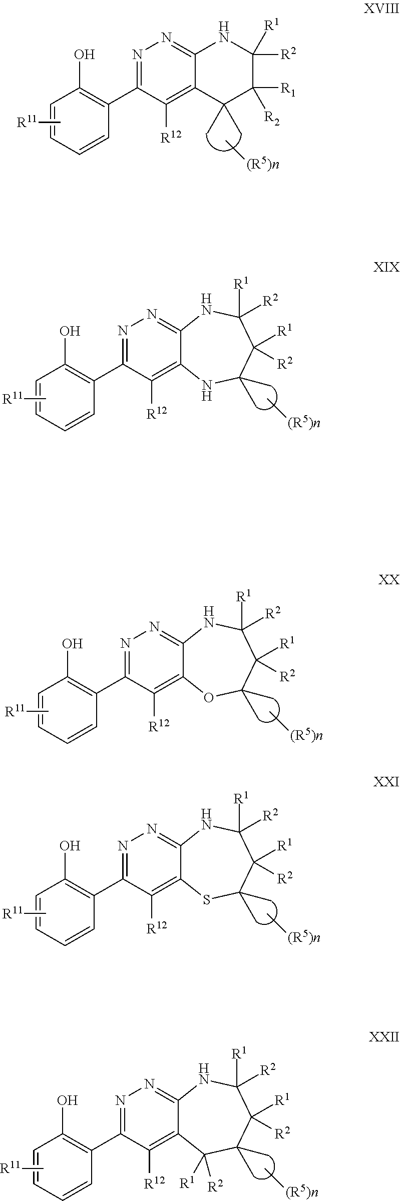

or a pharmaceutically acceptable salt or solvate thereof; wherein L is directly attached to ring A in any of the above formulae or wherein L is attached to ring A by an R⁵ group in any of the above formulae; or wherein L is attached to A′ by an R¹ or R² group in any of the above formulae.

In some embodiments, the compounds are compounds of formula XIII. In some embodiments, the compounds are compounds of formula XIV. In some embodiments, the compounds are compounds of formula XV. In some embodiments, the compounds are compounds of formula XVI. In some embodiments, the compounds are compounds of formula XVII. In some embodiments, the compounds are compounds of formula XVIII. In some embodiments, the compounds are compounds of formula XIX. In some embodiments, the compounds are compounds of formula XX. In some embodiments, the compounds are compounds of formula XXI. In some embodiments, the compounds are compounds of formula XXII.

In some aspects, the disclosure is directed to compounds of formula XXIII, formula XXIV, formula XXV, formula XXVI, or formula XXVII.

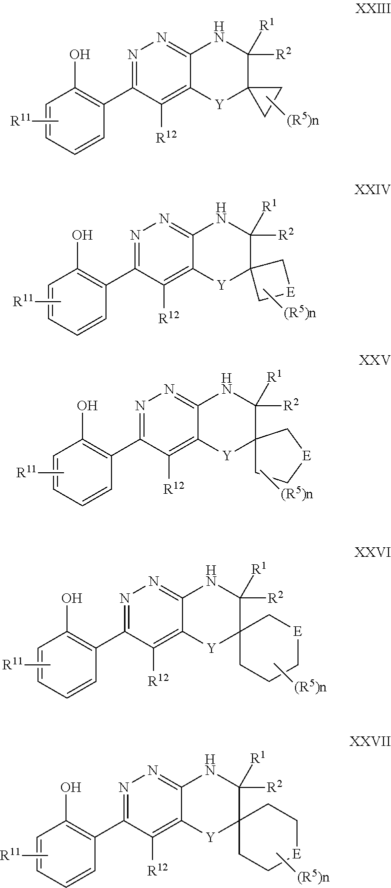

or a pharmaceutically acceptable salt or solvate thereof;
wherein E is CR¹R², O, C(O)NR^{c}R^{d}, -L-B, C(R¹)(L-B), or NR^{b}; wherein when E is not N-L-B or C(R¹)(L-B), L is attached to ring A by an R¹, R², or R⁵ group included in E in any of the above formulae; or wherein L is attached to A′ by an R¹ or R² group in any of the above formulae; and wherein Y is a bond, NR^{a}, O, S or CR¹R².

In some embodiments, the compounds are compounds of formula XXIII

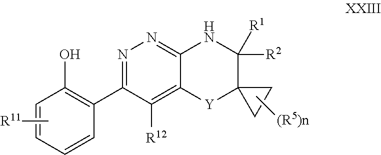

or a pharmaceutically acceptable salt or solvate thereof,
wherein L is directly attached to ring A or L is attached to ring A by an R⁵ group in the above formula; or wherein L is attached to A′ by an R¹ or R² group in the above formula and wherein Y is a bond, NR^{a}, O, S or CR¹R².

In some embodiments, Y in formula XXIII is a bond. In some embodiments, Y in formula XXIII is NR^{a}. In some embodiments, Y in formula XXIII is O. In some embodiments, Y in formula XXIII is S. In some embodiments, Y in formula XXIII is CR¹R².

In some embodiments, L in formula XXIII is attached to A′ by an R¹ group. In some embodiments, L in formula XXIII is attached to A′ by an R² group. In some embodiments, L in formula XXIII is attached to ring A by an R⁵ group. In some embodiments, L in formula XXIII is directly attached to ring A.

In some embodiments, the compounds are compounds of formula XXIV

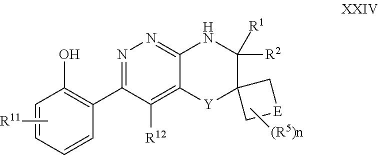

or a pharmaceutically acceptable salt or solvate thereof;
wherein E is CR¹R², O, C(O)NR^{c}R^{d}, N-L-B, C(R¹)(L-B), or NR^{b}; wherein when E is not N-L-B, C(R¹)(L-B), L is attached to ring A by an R¹, R² or R⁵ group included in E in the above formula; or wherein L is attached to A′ by an R¹ or R² group in the above formula; and wherein Y is a bond, NR^{a}, O, S or CR¹R².

In some embodiments, E in formula XXIV is CR¹R². In some embodiments, E in formula XXIV is O. In some embodiments, E in formula XXIV is C(O)NR^{c}R^{d}. In some embodiments, E in formula XXIV is N-L-B. In some embodiments, E in formula XXIV is C(R¹)(L-B). In some embodiments, E in formula XXIV is NR^{b}.

In some embodiments, when E in formula XXIV is not N-L-B, C(R¹)(L-B), L in formula XXIV is attached to ring A by an R⁵ group. In some embodiments, when E in formula XXIV is not N-L-B, C(R¹)(L-B), L in formula XXIV is attached to ring A by an R¹ group included in E. In some embodiments, when E in formula XXIV is not N-L-B, C(R¹)(L-B), L in formula XXIV is attached to ring A by an R² group included in E. In some embodiments, when E in formula XXIV is not N-L-B, C(R¹)(L-B), L in formula XXIV is attached to A′ by an R¹ group. In some embodiments, when E in formula XXIV is not N-L-B, C(R¹)(L-B), L in formula XXIV is attached to A′ by an R² group.

In some embodiments, Y in formula XXIV is a bond. In some embodiments, Y in formula XXIV is NR^{a}. In some embodiments, Y in formula XXIV is O. In some embodiments, Y in formula XXIV is S. In some embodiments, Y in formula XXIV is CR¹R².

In some embodiments, the compounds are compounds of formula XXV

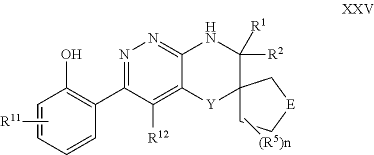

or a pharmaceutically acceptable salt or solvate thereof;
wherein E is CR¹R², O, C(O)NR^{c}R^{d}, N-L-B, C(R¹)(L-B), or NR^{b}; wherein when E is not N-L-B, C(R¹)(L-B), L is attached to ring A by an R¹, R² or R⁵ group included in E in the above formula; or wherein L is attached to A′ by an R¹ or R² group in the above formula; and wherein Y is a bond, NR^{a}, O, S or CR¹R².

In some embodiments, E in formula XXV is CR¹R². In some embodiments, E in formula XXV is O. In some embodiments, E in formula XXV is C(O)NR^{c}R^{d}. In some embodiments, E in formula XXV is N-L-B. In some embodiments, E in formula XXV is C(R¹)(L-B). In some embodiments, E in formula XXV is NR^{b}.

In some embodiments, when E in formula XXV is not N-L-B, C(R¹)(L-B), L in formula XXV is attached to ring A by an R⁵ group. In some embodiments, when E in formula XXV is not N-L-B, C(R¹)(L-B), L in formula XXV is attached to ring A by an R¹ group included in E. In some embodiments, when E in formula XXV is not N-L-B, C(R¹)(L-B), L in formula XXV is attached to ring A by an R² group included in E. In some embodiments, when E in formula XXV is not N-L-B, C(R¹)(L-B), L in formula XXV is attached to A′ by an R¹ group. In some embodiments, when E in formula XXV is not N-L-B, C(R¹)(L-B), L in formula XXV is attached to A′ by an R² group.

In some embodiments, Y in formula XXV is a bond. In some embodiments, Y in formula XXV is NR^{a}. In some embodiments, Y in formula XXV is O. In some embodiments, Y in formula XXV is S. In some embodiments, Y in formula XXV is CR¹R².

In some embodiments, the compounds are compounds of formula XXVI

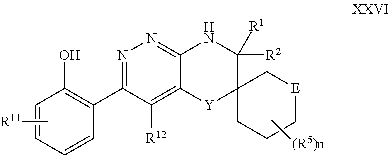

or a pharmaceutically acceptable salt or solvate thereof,
wherein E is CR¹R², O, C(O)NR^{c}R^{d}, N-L-B, C(R¹)(L-B), or NR^{b}; wherein when E is not N-L-B, C(R¹)(L-B), L is attached to ring A by an R¹, R² or R⁵ group in the above formula; or wherein L is attached to A′ by an R¹ or R² group in the above formula; and wherein Y is a bond, NR^{a}, O, S or CR¹R².

In some embodiments, E in formula XXVI is CR¹R². In some embodiments, E in formula XXVI is O. In some embodiments, E in formula XXVI is C(O)NR^{c}R^{d}. In some embodiments, E in formula XXVI is N-L-B. In some embodiments, E in formula XXVI is C(R¹)(L-B). In some embodiments, E in formula XXVI is NR^{b}.

In some embodiments, when E in formula XXVI is not N-L-B, C(R¹)(L-B), L in formula XXVI is attached to ring A by an R⁵ group. In some embodiments, when E in formula XXVI is not N-L-B, C(R¹)(L-B), L in formula XXVI is attached to ring A by an R¹ group included in E. In some embodiments, when E in formula XXVI is not N-L-B, C(R¹)(L-B), L in formula XXVI is attached to ring A by an R² group included in E. In some embodiments, when E in formula XXVI is not N-L-B, C(R¹)(L-B), L in formula XXVI is attached to A′ by an R¹ group. In some embodiments, when E in formula XXVI is not N-L-B, C(R¹)(L-B), L in formula XXVI is attached to A′ by an R² group.

In some embodiments, Y in formula XXVI is a bond. In some embodiments, Y in formula XXVI is NR^{a}. In some embodiments, Y in formula XXVI is O. In some embodiments, Y in formula XXVI is S. In some embodiments, Y in formula XXVI is CR¹R².

In some embodiments, the compounds are compounds of formula XXVII

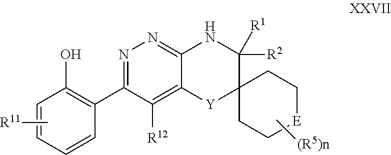

or a pharmaceutically acceptable salt or solvate thereof;
wherein E is CR¹R², O, C(O)NR^{c}R^{d}, N-L-B, C(R¹)(L-B), or NR^{b}; wherein when E is not N-L-B, C(R¹)(L-B), L is attached to ring A by an R¹, R² or R⁵ group included in E in the above formula; or wherein L is attached to A′ by an R¹ or R² group in the above formula; and wherein Y is a bond, NR^{a}, O, S or CR¹R².

In some embodiments, E in formula XXVII is CR¹R². In some embodiments, E in formula XXVII is O. In some embodiments, E in formula XXVII is C(O)NR^{c}R^{d}. In some embodiments, E in formula XXVII is N-L-B. In some embodiments, E in formula XXVII is C(R¹)(L-B). In some embodiments, E in formula XXVII is NR^{b}.

In some embodiments, when E in formula XXVII is not N-L-B, C(R¹)(L-B), L in formula XXVII is attached to ring A by an R⁵ group. In some embodiments, when E in formula XXVII is not N-L-B, C(R¹)(L-B), L in formula XXVII is attached to ring A by an R¹ group included in E. In some embodiments, when E in formula XXVII is not N-L-B, C(R¹)(L-B), L in formula XXVII is attached to ring A by an R² group included in E. In some embodiments, when E in formula XXVII is not N-L-B, C(R¹)(L-B), L in formula XXVII is attached to A′ by an R¹ group. In some embodiments, when E in formula XXVII is not N-L-B, C(R¹)(L-B), L in formula XXVII is attached to A′ by an R² group.

In some embodiments, Y in formula XXVII is a bond. In some embodiments, Y in formula XXVII is NR^{a}. In some embodiments, Y in formula XXVII is O. In some embodiments, Y in formula XXVII is S. In some embodiments, Y in formula XXVII is CR¹R².

In some embodiments, the compounds are compounds of formula XXVIII:

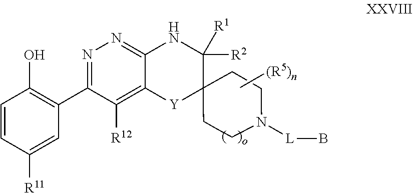

or a pharmaceutically acceptable salt or solvate thereof,
wherein o is 0, 1 or 2; and wherein Y is a bond, NR^{a}, O, S or CR¹R².

In some embodiments, o in formula XXVIII is 0. In some embodiments, o in formula XXVIII is 1. In some embodiments, o in formula XXVIII is 2.

In some embodiments, Y in formula XXVIII is a bond. In some embodiments, Y in formula XXVIII is NR^{a}. In some embodiments, Y in formula XXVIII is NH. In some embodiments, Y in formula XXVIII is O. In some embodiments, Y in formula XXVIII is S. In some embodiments, Y in formula XXVIII is CR¹R². In some embodiments, Y in formula XXVIII is CH₂.

In some embodiments, R¹² in formula XXVIII is H. In some embodiments, R¹¹ in formula XXVIII is H. In some embodiments, R¹¹ in formula XXVIII is F. In some embodiments, R¹ in formula XXVIII is H. In some embodiments, R² in formula XXVIII is H. In some embodiments, R⁵ in formula XXVIII is H. In some embodiments, R¹ and R² in formula XXVIII combine to form a carbonyl. In some embodiments, each of R¹, R², R⁵, R¹¹ and R¹² in formula XXVIII is H.

In some embodiments, the compounds are compounds of formula XXIX:

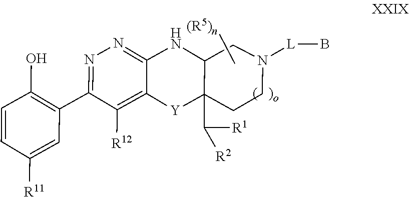

or a pharmaceutically acceptable salt or solvate thereof,
wherein o is 0, 1 or 2; and wherein Y is a bond, NR^{a}, O, S or CR¹R².

In some embodiments, o in formula XXIX is 0. In some embodiments, o in formula XXIX is 1. In some embodiments, o in formula XXIX is 2.

In some embodiments, Y in formula XXIX is a bond. In some embodiments, Y in formula XXIX is NR^{a}. In some embodiments, Y in formula XXIX is NH. In some embodiments, Y in formula XXIX is O. In some embodiments, Y in formula XXIX is S. In some embodiments, Y in formula XXIX is CR¹R². In some embodiments, Y in formula XXIX is CH₂.

In some embodiments, R¹² in formula XXIX is H. In some embodiments, R¹¹ in formula XXIX is H. In some embodiments, R¹¹ in formula XXIX is F. In some embodiments, R¹ in formula XXIX is H. In some embodiments, R² in formula XXIX is H. In some embodiments, R⁵ in formula XXIX is H. In some embodiments, R¹ and R² in formula XXIX combine to form a carbonyl. In some embodiments, each of R¹, R², R⁵, R¹¹ and R¹² in formula XXIX is H.

In some aspects, L in formula I, or in any other of formulae of II-XXIX, is a covalent bond, or chemical moiety that links A′ and B.

In some embodiments, L in formula I, or in any other of formulae of II-XXIX, is a covalent bond.

In other embodiments, L in formula I, or in any other of formulae of II-XXIX, is a chemical moiety that links A′ and B.

Chemical moieties that are used to link A′ and B moieties are known in the art. These moieties are sometimes referred to as “linkers” in the art. In some embodiments, L in formula I, or in any other of formulae of II-XXIX, is a chemical moiety that is used to link A′ and B that is known in the art.

In some embodiments, L in formula I, or in any other of formulae of II-XXIX, is a chemical moiety that is used to link A′ and B as described in U.S. Patent Application Publication No. 2019/0300521, the entirety of which is incorporated by reference herein.

In other embodiments, L in formula I, or in any other of formulae of II-XXIX, is a chemical moiety that is used to link A′ and B as described in U.S. Patent Application Publication No. 2019/0255066, the entirety of which is incorporated by reference herein.

In other embodiments, L in formula I, or in any other of formulae of II-XXIX, is a chemical moiety that is used to link A′ and B as described in WO 2019/084030, the entirety of which is incorporated by reference herein.

In other embodiments, L in formula I, or in any other of formulae of II-XXIX, is a chemical moiety that is used to link A′ and B as described in WO 2019/084026, the entirety of which is incorporated by reference herein.

In some embodiments, L in formula I, or in any other of formulae of II-XXIX, is a chemical structural unit represented by the formula:

-(A)_{q}-,

wherein:
q is an integer from 1 to 14;
each A is independently selected from the group consisting of CR^{1a}R^{1b}, O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR(CR^{1a}R^{1b})₁₋₄, NR^{1c}C(═NCN)NR^{1d}NR^{1c}C(═NCN), NR^{1c}C(═CNO₂)NR^{1d}, 3-11 membered cycloalkyl, optionally substituted with 0-6 R^{1a} and/or R^{1b} groups, 3-11 membered heteocyclyl optionally substituted with 0-6 R^{1a} and/or R^{1b} groups, aryl optionally substituted with 0-6 R^{1a} and/or R^{1b} groups, heteroaryl optionally substituted with 0-6 R^{1a} and/or R^{1b} groups,
and R^{1a}, R^{1b}, R^{1c}, R^{1d} and R^{1e} are each independently, —H, D, -halo, —C₁-C₈alkyl, —C₁-C₆haloalkyl, —O—C₁-C₈alkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; or where the context permits, R^{1a} or R^{1b}, are linked to other groups, or to each other, to form a cycloalkyl and/or a heterocyclyl moiety, optionally substituted with 0-4 R^{1e} groups.

In these embodiments, q represents the number of connected A groups. For example, when q=1, -(A)_{q}- is -A₁-; when q=2, -(A)_{q}- is -A₁-A₂-; when q=3, -(A)_{q}- is -A₁-A₂-A₃-; when q=4, -(A)_{q}- is -A₁-A₂-A₃-A₄-; when q=5, -(A)_{q}- is -A₁-A₂-A₃-A₄-A₅-; when q=6, -(A)_{q}- is -A₁-A₂-A₃-A₄-A₅-A₆-; when q=7, -(A)_{q}- is -A₁-A₂-A₃-A₄-A₅-A₆-A₇-; when q=8, -(A)_{q}- is -A₁-A₂-A₃-A₄-A₅-A₆-A₇-A₈-; when q=9, -(A)_{q}- is -A₁-A₂-A₃-A₄-A₅-A₆-A₇-A₈-A₉-; when q=10, -(A)_{q}- is -A₁-A₂-A₃-A₄-A₅-A₆-A₇-A₈-A₉-A₁₀-; when q=11, -(A)_{q}- is -A₁-A₂-A₃-A₄-A₅-A₆-A₇-A₈-A₉-A₁₀-A₁₁-; when q=12, -(A)_{q}- is -A₁-A₂-A₃-A₄-A₅-A₆-A₇-A₈-A₉-A₁₀-A₁₁-A₁₂-; when q=13, -(A)_{q}- is -A₁-A₂-A₃-A₄-A₅-A₆-A₇-A₈-A₉-A₁₀-A₁₁-A₁₂-A₁₃-; and when q=14, -(A)_{q}- is -A₁-A₂-A₃-A₄-A₅-A₆-A₇-A₈-A₉-A₁₀-A₁₁-A₁₂-A₁₃-A₁₄-.

In some embodiments, q=4 and L is a chemical moiety represented by the formula: -A₁-A₂-A₃-A₄-, wherein each of A₁₋₄ is independently selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{b})₁₋₄, —(CR^{1a}R^{1b})₁₋₄S(CR^{1a}R^{1b})₁₋₄, —(CR^{1a}R^{1b})₁₋₄NR(CR^{1a}R^{1b})₁₋₄, optionally substituted 3-11 membered cycloalkyl, 3-11 membered heterocyclyl, aryl, and heteroaryl;
wherein R^{1a} and R^{1b} are each independently selected from the group consisting of —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and
R^{1c} and R^{1d} are each independently selected from the group consisting of H, D, optionally substituted C₁₋₄ alkyl, C₃₋₈ cyclcoalkyl, C₃₋₈ heterocyclcoalkyl, aryl, or heteroaryl.

In other embodiments, q=3 and L is a chemical moiety represented by the formula: -A₁-A₂-A₃-, wherein each of A₁₋₃ is independently selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR(CR^{1a}R^{1b})₁₋₄, optionally substituted 3-11 membered cycloalkyl, 3-11 membered heterocyclyl, aryl, and heteroaryl;
wherein R^{1a} and R^{1b} are each independently selected from the group consisting of —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and
R^{1c} and R^{1d} are each independently selected from the group consisting of H, D, optionally substituted C₁₋₄ alkyl, C₃₋₈ cyclcoalkyl, C₃₋₈ heterocyclcoalkyl, aryl, or heteroaryl.

In other embodiments, q=2 and L is a chemical moiety represented by the formula: -A₁-A₂-, wherein each of A₁₋₂ is independently selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR(CR^{1a}R^{1b})₁₋₄, optionally substituted 3-11 membered cycloalkyl, 3-11 membered heterocyclyl, aryl, and heteroaryl;
wherein R^{1a} and R^{1b} are each independently selected from the group consisting of —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and
R^{1c} and R^{1d} are each independently selected from the group consisting of H, D, optionally substituted C₁₋₄ alkyl, C₃₋₈ cyclcoalkyl, C₃₋₈ heterocyclcoalkyl, aryl, or heteroaryl.

In other embodiments, q=1 and L is a chemical moiety represented by the formula: -A₁-, wherein A₁ is selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR(CR^{1a}R^{1b})₁₋₄, optionally substituted 3-11 membered cycloalkyl, 3-11 membered heterocyclyl, aryl, and heteroaryl;
wherein R^{1a} and R^{1b} are each independently selected from the group consisting of —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and
R^{1c} and R^{1d} are each independently selected from the group consisting of H, D, optionally substituted C₁₋₄ alkyl, C₃₋₈ cyclcoalkyl, C₃₋₈ heterocyclcoalkyl, aryl, or heteroaryl.

In some embodiments, L is a covalent bond, 3-11 membered cycloalkyl optionally substituted with 0-6 R^{1a} and/or R^{1b} groups, 3-11 membered heterocyclyl optionally substituted with 0-6 R^{1a} and/or R^{1b} groups, —(CR^{1a}R^{1b})₁₋₅, —(CR^{1a}═CR^{1b})—, —(CR^{1a}R^{1b})₁₋₅-A- wherein A is O, S, or NR^{1c}, —(CR^{1a}R^{1b})₁₋₅-A-(CR^{1a}R^{1b})₁₋₅— wherein A is O, S, or NR^{1c}, —(CR^{1a}R^{1b})₁₋₅-A-(CR^{1a}R^{1b})¹⁻⁵-A- wherein A is O, S, or NR^{1c}, —(CR^{1a}R^{1b})₁₋₅—(CR^{1a}═CR^{1b})—(CR^{1a}R^{1b})₁₋₅—, —(CR^{1a}R^{1b})₁₋₅—(CR^{1a}═CR^{1b})—(CR^{1a}R^{1b})₁₋₅-A- wherein A is O, S, or NR^{1c}, —(CR^{1a}R^{1b})₁₋₅—(C≡C)—(CR^{1a}R^{1b})₁₋₅, —(CR^{1a}R^{1b})₁₋₅—(C≡C)—(CR^{1a}R^{1b})₁₋₅-A- wherein A is O, S, or NR^{1c}, —(C≡C)—(CR^{1a}R^{1b})₁₋₅-A-(CR^{1a}R^{1b})₁₋₅— wherein A is O, S, or NR^{1c}, —(C≡C)—(CR^{1a}R^{1b})₁₋₅, —(CR^{1a}R^{1b})₁₋₅-(3-11 membered cycloalkyl optionally substituted with 0-6 R^{1a} and/or R^{1b} groups)-, —(CR^{1a}R^{1b})₁₋₅-(3-11 membered heterocyclyl optionally substituted with 0-6 R^{1a} and/or R^{1b} groups)-, -(3-11 membered cycloalkyl optionally substituted with 0-6 R^{1a} and/or R^{1b} groups)-(CR^{1a}R^{1b})₁₋₅—, -(3-11 membered heterocyclyl optionally substituted with 0-6 R^{1a} and/or R^{1b} groups) —(CR^{1a}R^{1b})₁₋₅—, —(CR^{1a}R^{1b})₁₋₅-(3-11 membered cycloalkyl optionally substituted with 0-6 R^{1a} and/or R^{1b} groups)-A-, —(CR^{1a}R^{1b})₁₋₅-(3-11 membered heterocyclyl optionally substituted with 0-6 R^{1a} and/or R^{1b} groups)-A-, —(CR^{1a}R^{1b})₁₋₅-(3-11 membered cycloalkyl optionally substituted with 0-6 R^{1a} and/or R^{1b} groups)-(CR^{1a}R^{1b})₁₋₅, —(CR^{1a}R^{1b})₁₋₅-(3-11 membered cycloalkyl optionally substituted with 0-6 R^{1a} and/or R^{1b} groups)-A- wherein A is O, S, or NR^{1c}, —(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}, —(CR^{1a}R^{1b})₁₋₅-A-(3-11 membered cycloalkyl optionally substituted with 0-6 R^{1a} and/or R^{1b} groups)- wherein A is O, S, or NR^{1c}, —(CR^{1a}R^{1b})₁₋₅-(3-11 membered heterocyclyl optionally substituted with 0-6 R^{1a} and/or R^{1b} groups)-(CR^{1a}R^{1b})₁₋₅, —(CR^{1a}R^{1b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}, —(CR^{1a}R^{1b})₁₋₅-(3-11 membered heterocyclyl optionally substituted with 0-6 R^{1a} and/or R^{1b} groups)-A- wherein A is O, S, or NR^{1c}, —(CR^{1a}R^{1b})₁₋₅-A-(3-11 membered heterocyclyl optionally substituted with 0-6 R^{1a} and/or R^{1b} groups)- wherein A is O, S, or NR^{1c}, —(CR^{1a}R^{1b})₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}, —(CR^{1a}R^{1b})₁₋₅-A-(3-11 membered cycloalkyl optionally substituted with 0-6 R^{1a} and/or R^{1b} groups)- wherein A is O, S, or NR^{1c}, —(CR^{1a}R^{1b})₁₋₅-A-(3-11 membered cycloalkyl optionally substituted with 0-6 R^{1a} and/or R^{1b} groups)-(CR^{1a}R^{1b})₁₋₅-A- wherein each A is independently O, S, or NR^{1c}, —(CR^{1a}R^{1b})₁₋₅-A-(3-11 membered heterocyclyl optionally substituted with 0-6 R^{1a} and/or R^{1b} groups)-(CR^{1a}R^{1b})₁₋₅-A- wherein each A is independently O, S, or NR^{1c}, —(CR^{1a}R^{1b})₁₋₅-A-(CR^{1a}R^{1b})₁₋₅-A- wherein A is O, S, or NR^{1c}, —(CR^{1a}R^{1b})₁₋₅-A-(CR^{1a}R^{1b})₁₋₅-A-(CR^{1a}R^{1b})₁₋₅-A-(CR^{1a}R^{1b})₁₋₅-A- wherein A is O, S, or NR^{1c}, —(CR^{1a}R^{1b})₁₋₅-A-(CO) wherein A is O, S, or NR^{1c}, —(CR^{1a}R^{1b})₁₋₅—(CR^{1a}═CR^{1b})—(CR^{1a}R^{1b})₁₋₅-A-(CO)— wherein A is O, S, or NR^{1c}, —(CR^{1a}R^{1b})₁₋₅—(C≡C)—(CR^{1a}R^{1b})₁₋₅-A-(CO)— wherein A is O, S, or NR^{1c}, —(CR^{1a}R^{1b})₁₋₅-(3-11 membered cycloalkyl optionally substituted with 0-6 R^{1a} and/or R^{1b} groups)-(CR^{1a}R^{1b})₁₋₅-A-(CO)— wherein A is O, S, or NR^{1c}, —(CR^{1a}R^{1b})₁₋₅-A-(CO)-(3-11 membered cycloalkyl optionally substituted with 0-6 R^{1a} and/or R^{1b} groups)- wherein A is O, S, or NR^{1c}, —(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)— wherein A is O, S, or NR^{1c}, —(CR^{1a}R^{1b})₁₋₅-A-(CO)-(3-11 membered heterocyclyl optionally substituted with 0-6 R^{1a} and/or R^{1b} groups)- wherein A is O, S, or NR^{1c}, —(CR^{1a}R^{1b})₁₋₅-A-(3-11 membered cycloalkyl optionally substituted with 0-6 R^{1a} and/or R^{1b} groups)-A-(CO)— wherein each A is independently O, S, or NR^{1c}, -(3-11 membered cycloalkyl optionally substituted with 0-6 R^{1a} and/or R^{1b} groups)-CO—(CR^{1a}R^{1b})₁₋₅-A- wherein A is O, S, or NR^{1c}, —(CR^{1a}R^{1b})₁₋₅-(3-11 membered cycloalkyl optionally substituted with 0-6 R^{1a} and/or R^{1b}groups)-(CR¹R^{1b})₁₋₅-A-(CO)— wherein A is O, S, or NR^{1c}, —(CR^{1a}R^{1b})₁₋₅-(3-11 membered heterocyclyl optionally substituted with 0-6 R^{1a} and/or R^{1b} groups)-(CR^{1a}R^{1b})₁₋₅-A-(CO)- wherein A is O, S, or NR^{1c}, -(3-11 membered cycloalkyl optionally substituted with 0-6 R^{1a} and/or R^{1b} groups)-(CR^{1a}R^{1b})₁₋₅—, or -(3-11 membered heterocyclyl optionally substituted with 0-6 R^{1a} and/or R^{1b} groups)-(CR^{1a}R^{1b})₁₋₅—.

In some embodiments, L is —CR^{1a}═CR^{1b}—, such as, for example, —CH═CH—.

In some embodiments, L is —(CR^{1a}R^{1b})₁₋₅, for example —(CH₂)₁₋₅—, —CH₂—, —CH₂CH₂CH₂— and the like.

In some embodiments, L is —(CR^{1a}R^{1b})₁₋₅-A- wherein A is O, S, or NR^{1c}, such as for example, —(CH₂)₁₋₅—O—, —(CH₂)₁₋₅—S—, —(CH₂)₁₋₅—NH—, or —(CH₂)₀₋₂—(C(CH₃)₂)—(CH₂)₀₋₂—O—.

In other embodiments, L is —(CR^{1a}R^{1b})₁₋₅-A-(CR^{1a}R^{1b})₁₋₅— wherein A is O, S, or NR^{1c}, such as, for example, —(CH₂)₁₋₅—O—(CH₂)₁₋₅—, —(CH₂)₁₋₅—S—(CH₂)₁₋₅—, —(CH₂)₁₋₅—NH—(CH₂)₁₋₅—.

In some embodiments, L is —(C≡C)—(CR^{1a}R^{1b})₁₋₅, such as, for example, —(C≡C)—(CH₂)₂—, and the like.

In some embodiments, L is —(CR^{1a}R^{1b})₁₋₅-(3-11 membered cycloalkyl optionally substituted with 0-6 R^{1a} and/or R^{1b} groups)-, such as, for example, —CH₂-cyclobutyl-.

In some embodiments, L is —(CR^{1a}R^{1b})₁₋₅-(3-11 membered cycloalkyl optionally substituted with 0-6 R^{1a} and/or R^{1b} groups)-(CR^{1a}R^{1b})₁₋₅, such as, for example, —CH₂-cyclobutyl-CH₂— and the like.

In some embodiments, L is —(CR^{1a}R^{1b})₁₋₅-(3-11 membered heterocyclyl optionally substituted with 0-6 R^{1a} and/or R^{1b} groups)-(CR^{1a}R^{1b})₁₋₅, such as, for example, —CH₂-azetidinyl-CH₂—.

In some embodiments, L is —(CR^{1a}R^{1b})₁₋₅-(3-11 membered heterocyclyl optionally substituted with 0-6 R^{1a} and/or R^{1b} groups)-, such as, for example, —CH₂-azetidinyl-.

In some embodiments, L is -(3-11 membered heterocyclyl optionally substituted with 0-6 R^{1a} and/or R^{1b} groups)-(CR^{1a}R^{1b})₁₋₅—, such as, for example, -azetidinyl-CH₂—, -pyrolidnyl-CH₂—, -piperidinyl-CH₂—, and the like.

In some embodiments, L is —(CR^{1a}R^{1b})₁₋₅-(3-11 membered cycloalkyl optionally substituted with 0-6 R^{1a} and/or R^{1b} groups)-(CR^{1a}R^{1b}) is-A- wherein A is O, S, or NR^{1c}, such as, for example, —CH₂-cyclopropyl-CH₂—O—, and the like.

In some embodiments, L is —(CR^{1a}R^{1b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}, such as, for example, —CH₂-piperidinyl-CH₂CH₂—O—, and the like.

In some embodiments, L is —(CR^{1a}R^{1b})₁₋₅-(3-11 membered heterocyclyl optionally substituted with 0-6 R^{1a} and/or R^{1b} groups)-A- wherein A is O, S, or NR^{1c}, such as, for example, —CH₂-azetidinyl-O—, and the like.

In some embodiments, L is —(CR^{1a}R^{1b})₁₋₅-A-(3-11 membered heterocyclyl optionally substituted with 0-6 R^{1a} and/or R^{1b} groups)- wherein A is O, S, or NR^{1c}, such as, for example, —CH₂—O-azetidinyl-, —CH₂—NH-azetidinyl-, and the like.

In other embodiments, L is —(CR^{1a}R^{1b})₁₋₅-A-(3-11 membered cycloalkyl optionally substituted with 0-6 R^{1a} and/or R^{1b} groups)- wherein A is O, S, or NR^{1c}, such as —CH₂—O-cyclobutylene-, —CH₂—NH-cyclobutylene-, and the like.

In some embodiments, L is —(CR^{1a}R^{1b})₁₋₅-A-(CR^{1a}R^{1b})₁₋₅-A- wherein A is O, S, or NR_{1c}, such as, for example, —CH₂—O—CH₂CH₂—O—.

In some aspects, the B group in the compounds of the disclosure is a small molecule E3 Ubiquitin Ligase binding moiety that binds a Von Hippel-Lindau E3 Ubiquitin Ligase (VHL). Such ULM moieties that bind to VHL are known to those of skill in the art. Methods of determining whether a small molecule binds a Von Hippel-Lindau E3 Ubiquitin Ligase are known in the art.

In some embodiments, the B group is a previously described E3 Ubiquitin Ligase binding moiety.

In some embodiments, the B group is an E3 Ubiquitin Ligase binding moiety described in U.S. Patent Application Publication No. 2019/0300521, the entirety of which is incorporated by reference herein.

In other embodiments, the B group is an E3 Ubiquitin Ligase binding moiety described in U.S. Patent Application Publication No. 2019/0255066, the entirety of which is incorporated by reference herein.

In other embodiments, the B group is an E3 Ubiquitin Ligase binding moiety described in WO 2019/084030, the entirety of which is incorporated by reference herein.

In other embodiments, the B group is an E3 Ubiquitin Ligase binding moiety described in WO 2019/084026, the entirety of which is incorporated by reference herein.

In some embodiments, the B group is a moiety having the Formula B-I

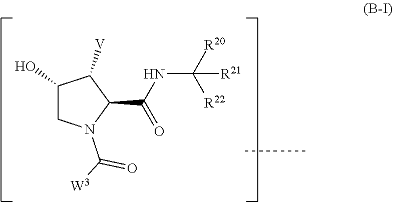

wherein
the dashed line ( [symbol] ) indicates the position of attachment of B-I to L;
V is H or F;
R²⁰ is optionally substituted phenyl, optionally substituted napthyl, or an optionally substituted 5-10 membered heteroaryl;
one of R²¹ or R²² is H, D, haloalkyl, optionally substituted alkyl, optionally substituted cycloalkyl, optionally substituted heterocycloalkyl, —COR^{g}, CONR^{h1}R^{h2}; the other of R²¹ or R²² is H or D;
or R²¹ and R²², together with the carbon atom to which they are both attached, form an optionally substituted 3-5 membered cycloalkyl, heterocyclyl;
W³ is an optionally substituted aryl, optionally substituted heteroaryl, or

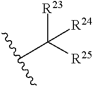

R²³ and R²⁴ are independently H, D, optionally substituted alkyl, optionally substituted cycloalkyl, or optionally substituted haloalkyl,
or R²³, R²⁴, and the carbon atom to which they are attached form an optionally substituted cycloalkyl or optionally substituted heterocyclyl;
R²⁵ is an optionally substituted heterocyclyl, optionally substituted heteroaryl, optionally substituted aryl, CONRⁱR^{j}, NRⁱR^{j},

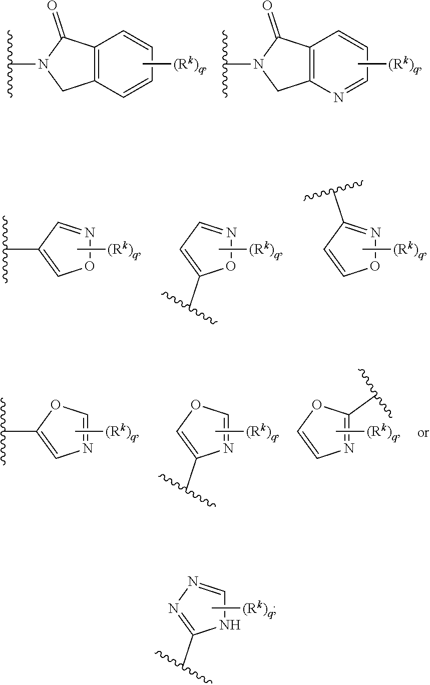

Rⁱ is selected from H or optionally substituted alkyl;
R^{j} is selected from H, —C(O)—* wherein * is a point of attachment to L, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (cycloalkyl)carbonyl, optionally substituted (heterocyclyl) carbonyl, or optionally substituted aralkyl;
each R^{k} is independently H, halo, optionally substituted alkoxy, cyano, optionally substituted alkyl, haloalkyl, or haloalkoxy;
each R^{g} is independently selected from H, optionally substituted alkyl or NR^{h1}R^{h2}.
each R^{h1} and R^{h2} is independently H, D, optionally substituted alkyl,
or R^{h1} and R^{h2} together with the nitrogen atom to which they are attached form a 4-7 membered heterocyclyl; and
q is 0, 1, 2, 3, or 4.

In some embodiments of B-I, V is H.

In other embodiments of B-I, V is F.

In some embodiments of B-I, R²⁰ is optionally substituted phenyl having the formula:

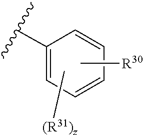

wherein
R³⁰ is H, D, halo, —CN, —OH, —NO₂, —NR^{h1}R^{h2}, —OR^{h1}, —CONR^{h1}R^{h2}, —NR^{h1}COR^{h2}, —SO₂NR^{h1}R^{h2}, —NR^{h1}SO₂R^{h2}, optionally substituted alkyl, optionally substituted alkoxyl, optionally substituted haloalkyl, optionally substituted haloalkoxy; optionally substituted aryl; optionally substituted heteroaryl; optionally substituted cycloalkyl; or optionally substituted heterocyclyl;
R³¹ is H, D, halo, CN, optionally substituted alkyl, optionally substituted haloalkyl, hydroxy, or optionally substituted haloalkoxy; and
z is 0, 1, 2, 3, or 4.

In some embodiments of B-I, R²⁰ is optionally substituted phenyl, R³⁰ is an optionally substituted heteroaryl.

In some embodiments of B-I, R²⁰ is optionally substituted phenyl, R³⁰ is

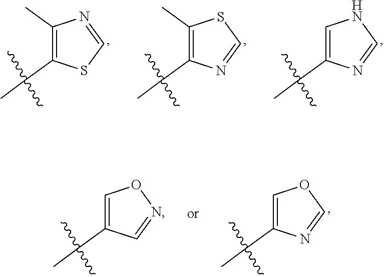

each optionally substituted.

In other embodiments of B-I, R²⁰ is optionally substituted phenyl, R³⁰ is

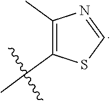 [SMILES: *c1scnc1C]

In other embodiments, R²⁰ is

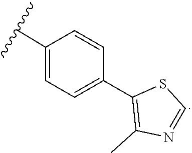 [SMILES: *c1ccc(-c2scnc2C)cc1]

In some embodiments of B-I, R²⁰ is optionally substituted phenyl, R³¹ is hydroxy, halogen, —NH(C₁-C₄alkyl), or C₁-C₆alkoxy, and z is 0, 1, 2, 3, or 4.

In some embodiments of B-I, one of R²¹ or R²² is H, and the other of R²¹ or R²² is H or optionally substituted alkyl.

In other embodiments of B-I, one of R²¹ or R²² is H, and the other of R²¹ or R²² is optionally substituted C₁-C₆alkyl.

In other embodiments of B-I, one of R²¹ or R²² is H, and the other of R²¹ or R²² is C₁-C₆alkyl.

In other embodiments of B-I, one of R²¹ or R²² is H, and the other of R²¹ or R²² is —CH₃.

In other embodiments of B-I, both R²¹ and R²² are H.

In some embodiments of B-I, W³ is

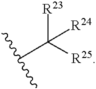

In some embodiments of B-I, R²³ is H.

In some embodiments of B-I, R²⁴ is H, or optionally substituted alkyl.

In some embodiments of B-I, R²⁴ is H.

In some embodiments of B-I, R²⁴ is optionally substituted alkyl.

In some embodiments of B-I, R²⁴ is optionally substituted C₁-C₆alkyl.

In some embodiments of B-I, R²⁴ is C₁-C₆alkyl.

In some embodiments of B-I, R²⁴ is C₁-C₆alk-OH, C₁-C₆alk-NH₂, —C₁-C₆alk-CONH—*, or —C₁-C₆alk-NHCO—* wherein * is a point of attachment to L.

In some embodiments of B-I, R²⁴ is -t-butyl or -isopropyl.

In some embodiments of B-I, R²⁵ is NR^{a}R^{b}.

In some embodiments of B-I, Rⁱ is H or optionally substituted alkyl.

In some embodiments of B-I, Rⁱ is H.

In some embodiments of B-I, R^{j} is H, optionally substituted alkyl, —C(O)—* wherein * is a point of attachment to L, optionally substituted (cycloalkyl)carbonyl, or optionally substituted alkylcarbonyl.

In some embodiments of B-I, R^{j} is optionally substituted alkylcarbonyl.

In some embodiments of B-I, R^{j} is —C(O)—* wherein * is a point of attachment to L.

In some embodiments of B-I, R²⁵ is CONRⁱR^{j}.

In some embodiments of B-I, R²⁵ is

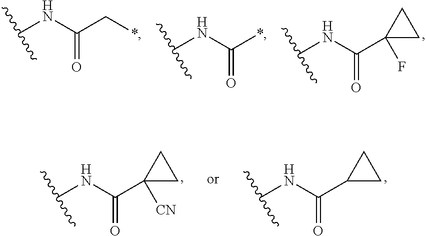

wherein * is a point of attachment to L.

In some embodiments of B-I, R²⁵ is

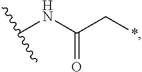

wherein * is a point of attachment to R¹.

In some embodiments of B-I, R²⁵ is

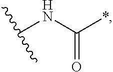

wherein * is a point of attachment to R¹.

In some embodiments of B-I, R²⁵ is

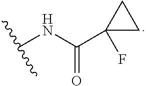 [SMILES: *NC(=O)C1(F)CC1]

In some embodiments of B-I, R²⁵ is —NH—* wherein * is a point of attachment to R¹.

In some embodiments of B-I, R²⁵ is optionally substituted heteroaryl.

In some embodiments of B-I, R²⁵ is

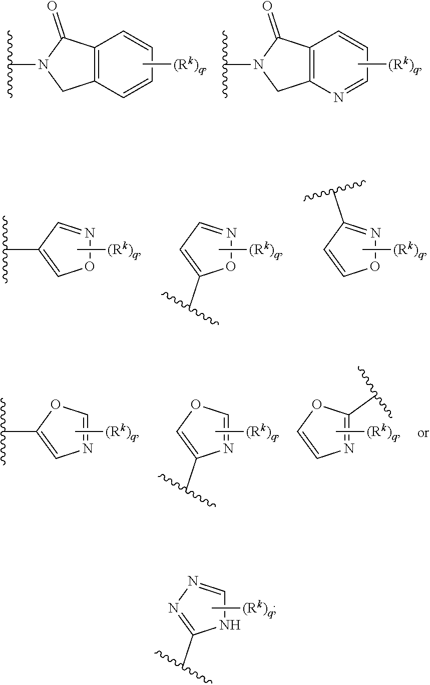

wherein each R^{k} is independently halo, optionally substituted alkoxy, cyano, optionally substituted alkyl, haloalkyl, or haloalkoxy, and q is 0, 1, or 2.

In some embodiments, R²⁵ is

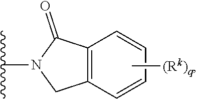

wherein each R^{k} is independently halo, optionally substituted alkoxy, cyano, optionally substituted alkyl, haloalkyl, or haloalkoxy, and q is 0, 1, or 2.

In some embodiments, R²⁵ is

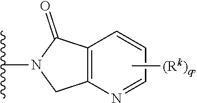

wherein each R^{k} is independently halo, optionally substituted alkoxy, cyano, optionally substituted alkyl, haloalkyl, or haloalkoxy, and q is 0, 1, or 2.

In some embodiments, R²⁵ is

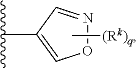

wherein each R^{k} is independently halo, optionally substituted alkoxy, cyano, optionally substituted alkyl, haloalkyl, or haloalkoxy, and q is 0, 1, or 2.

In some embodiments, R²⁵ is

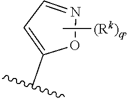

wherein each R^{k} is independently halo, optionally substituted alkoxy, cyano, optionally substituted alkyl, haloalkyl, or haloalkoxy, and q is 0, 1, or 2.

In some embodiments, R²⁵ is

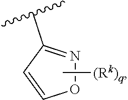

wherein each R^{k} is independently halo, optionally substituted alkoxy, cyano, optionally substituted alkyl, haloalkyl, or haloalkoxy, and q is 0, 1, or 2.

In some embodiments, R²⁵ is

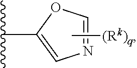

wherein each R^{k} is independently halo, optionally substituted alkoxy, cyano, optionally substituted alkyl, haloalkyl, or haloalkoxy, and q is 0, 1, or 2.

In some embodiments, R²⁵ is

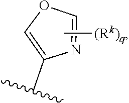

wherein each R^{k} is independently halo, optionally substituted alkoxy, cyano, optionally substituted alkyl, haloalkyl, or haloalkoxy, and q is 0, 1, or 2.

In some embodiments, R²⁵ is

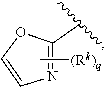

wherein each R^{k} is independently halo, optionally substituted alkoxy, cyano, optionally substituted alkyl, haloalkyl, or haloalkoxy, and q is 0, 1, or 2.

In some embodiments, R²⁵ is

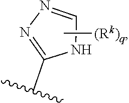

wherein each R^{k} is independently halo, optionally substituted alkoxy, cyano, optionally substituted alkyl, haloalkyl, or haloalkoxy, and q is 0, 1, or 2.

In some embodiments, B-I is a compound of formula:

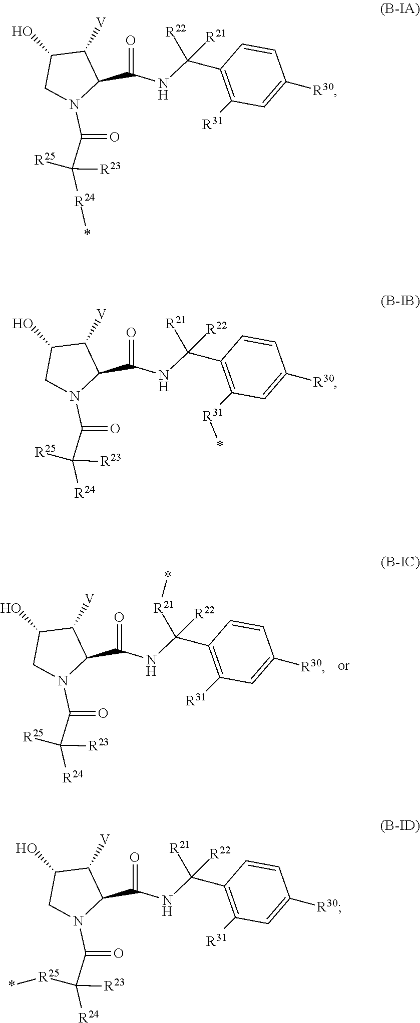

wherein * is a point of attachment to L.

In some embodiments of B-IA, B-IB B-IC, or B-ID, R³⁰ is optionally substituted

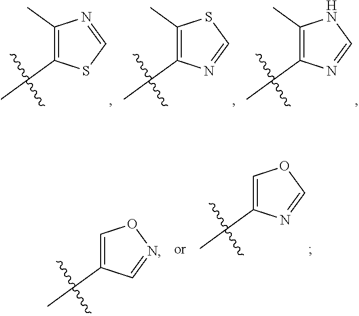

and
and R³¹ is H, D, hydroxy, halogen, aminoC₁₋₄alkyl, or C₁₋₄alkyloxy.

In some aspects, the compounds of Formula I are those having the formula IA-7 or IA-8:

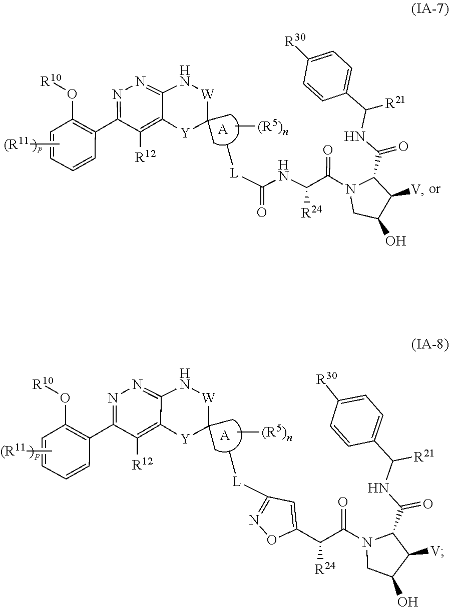

wherein R¹¹, R¹⁰, R⁵, R¹², R³⁰, R²⁴, R²¹, Y, W, V, p and n are as defined previously.

In some aspects, the compounds of Formula I are those having the formula IA-9, IA-10, IA-11 or IA-12:

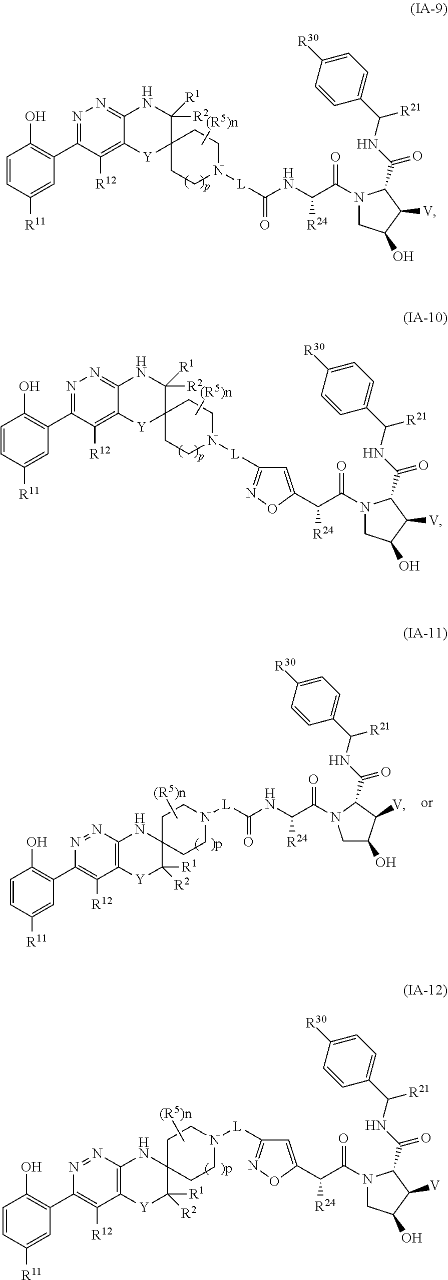

wherein R¹¹, R¹, R², R⁵, R¹², R³⁰, R²⁴, R²¹, Y, V, p and n are as defined previously.

In some embodiments of the compound of Formula IA-7, IA-8, IA-9, IA-10, IA-11 or IA-12, n=1. In other embodiments of the compound of Formula IA-7, IA-8, IA-9, IA-10, IA-11 or IA-12, n=2. In other embodiments of the compound of Formula IA-7, IA-8, IA-9, IA-10, IA-11 or IA-12, n=3.

In some embodiments of the compound of Formula IA-7, IA-8, IA-9, IA-10, IA-11 or IA-12, p=0. In some embodiments of the compound of Formula IA-7, IA-8, IA-9, IA-10, IA-11 or IA-12, p=1. In some embodiments of the compound of Formula IA-7, IA-8, IA-9, IA-10, IA-11 or IA-12, p=2.

In some embodiments, Y in formula IA-7, IA-8, IA-9, IA-10, IA-11 or IA-12 is a bond. In some embodiments, Y in formula IA-7, IA-8, IA-9, IA-10, IA-11 or IA-12 is NR^{a}. In some embodiments, Y in formula IA-7, IA-8, IA-9, IA-10, IA-11 or IA-12 is NH. In some embodiments, Y in formula IA-7, IA-8, IA-9, IA-10, IA-11 or IA-12 is O. In some embodiments, Y in formula IA-7, IA-8, IA-9, IA-10, IA-11 or IA-12 is S. In some embodiments, Y in formula IA-7, IA-8, IA-9, IA-10, IA-11 or IA-12 is CR¹R². In some embodiments, Y in formula IA-7, IA-8, IA-9, IA-10, IA-11 or IA-12 is CH₂.

In some embodiments, R¹² in formula IA-7, IA-8, IA-9, IA-10, IA-11 or IA-12 is H. In some embodiments, R¹¹ in formula IA-7, IA-8, IA-9, IA-10, IA-11 or IA-12 is H. In some embodiments, R¹¹ in formula IA-7, IA-8, IA-9, IA-10, IA-11 or IA-12 is F. In some embodiments, R¹ in formula IA-7, IA-8, IA-9, IA-10, IA-11 or IA-12 is H. In some embodiments, R² in formula IA-7, IA-8, IA-9, IA-10, IA-11 or IA-12 is H. In some embodiments, R¹ in formula IA-7, IA-8, IA-9, IA-10, IA-11 or IA-12 is H. In some embodiments, R¹ and R² in formula IA-7, IA-8, IA-9, IA-10, IA-11 or IA-12 combine to form a carbonyl. In some embodiments, each of R¹, R², R⁵, R¹¹ and R¹² in formula IA-7, IA-8, IA-9, IA-10, IA-11 or IA-12 is H.

In some embodiments of the compound of Formula IA-7, IA-8, IA-9, IA-10, IA-11 or IA-12, V=H. In other embodiments of the compound of Formula IA-7, IA-8, IA-9, IA-10, IA-11 or IA-12, V=F.

In some embodiments, R³⁰ in formula IA-7, IA-8, IA-9, IA-10, IA-11 or IA-12 is H. In some embodiments, R²¹ in formula IA-7, IA-8, IA-9, IA-10, IA-11 or IA-12 is H. In some embodiments, R²⁴ in formula IA-7, IA-8, IA-9, IA-10, IA-11 or IA-12 is H. In some embodiments, each of R³⁰, R²¹, and R²⁴ in formula IA-7, IA-8, IA-9, IA-10, IA-11 or IA-12 is H.

It will be apparent that the compounds of the invention, including all subgenera described herein, may have multiple stereogenic centers. As a result, there exist multiple stereoisomers (enantiomers and diastereomers) of the compounds (and subgenera described herein). The present disclosure contemplates and encompasses each stereoisomer of any compound of encompassed by the disclosure as well as mixtures of said stereoisomers.

Pharmaceutically acceptable salts and solvates of the compounds of the disclosure (including all subgenera described herein) are also within the scope of the disclosure.

Isotopic variants of the compounds of the disclosure (including all subgenera described herein) are also contemplated by the present disclosure.

### Pharmaceutical Compositions and Methods of Administration

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time. In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.00010% to approximately 50%, approximately 0.0010% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.0010% to approximately 10%, approximately 0.010% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 10%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition of the invention typically contains an active ingredient (e.g., a compound of the disclosure) of the present invention or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizers and adjuvants.

Described below are non-limiting exemplary pharmaceutical compositions and methods for preparing the same.

### Pharmaceutical Compositions for Oral Administration.

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (e.g., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP -phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof, polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-lOoleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, E-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%), 100%, or up to about 200%> by weight, based on the combined weight of the drug, and other excipients. If desired, very small amounts of solubilizer may also be used, such as 5%>, 2%>, 1%) or even less. Typically, the solubilizer may be present in an amount of about 1%> to about 100%, more typically about 5%> to about 25%> by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical Compositions for Topical (e.g. Transdermal) Delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins, 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation. Compounds can also be administered intraadiposally or intrathecally.

In some embodiments, the compounds or pharmaceutical composition of the present invention are administered by intravenous injection.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose. Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; U.S. Pat. Nos. 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly. The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc. Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

In certain embodiment, the present invention provides a pharmaceutical composition comprising a compound of bispecific formula, or pharmaceutically acceptable salt thereof.

In certain embodiment, the present invention provides a pharmaceutical composition comprising a compound of bispecific formula for use in degrading a target protein in a cell.

In certain embodiment, a method of degrading a target protein comprising administering to a cell therapeutically effective amount of a bispecific compound, or pharmaceutically acceptable salt, wherein the compound is effective for degrading the target protein.

In certain embodiment, the present invention provides a pharmaceutical composition comprising a compound of bispecific formula, for use in treating or preventing of a disease or disorder in which SMARCA2 and/or SMARCA4 plays a role.

In certain embodiment, the present invention provides a pharmaceutical composition comprising a compound of bispecific formula, for use in treating or preventing of a disease or disorder in which SWI/SNF mutations plays a role.

In certain embodiment, target proteins are SMARCA2, SMARCA4 and/or PBT.

In certain embodiment, target protein complex is SWI/SNF in a cell.

In certain embodiment, diseases or disorders dependent on SMARCA2 or SMARCA4 include cancers.

In certain embodiment, diseases or disorders dependent on SWI/SNF complex include cancers.

Exemplary cancers which may be treated by the present compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present disclosure include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

In certain further embodiment, the cancer is a SMARCA2 and/or SMARAC4-dependent cancer.

In certain embodiment, the present invention provides a pharmaceutical composition comprising a compound of bispecific formula for use in the diseases or disorders dependent upon SMARCA2 and/or SMARCA4 is cancer.

Compounds of the disclosure, as well as pharmaceutical compositions comprising them, can be administered to treat any of the described diseases, alone or in combination with a medical therapy. Medical therapies include, for example, surgery and radiotherapy (e.g., gamma-radiation, neutron beam radiotherapy, electron beam radiotherapy, proton therapy, brachytherapy, systemic radioactive isotopes).

In other aspects, compounds of the disclosure, as well as pharmaceutical compositions comprising them, can be administered to treat any of the described diseases, alone or in combination with one or more other agents.

In other methods, the compounds of the disclosure, as well as pharmaceutical compositions comprising them, can be administered in combination with agonists of nuclear receptors agents.

In other methods, the compounds of the disclosure, as well as pharmaceutical compositions comprising them, can be administered in combination with antagonists of nuclear receptors agents.

In other methods, the compounds of the disclosure, as well as pharmaceutical compositions comprising them, can be administered in combination with an anti-proliferative agent.

### Combination Therapies

For treating cancer and other proliferative diseases, the compounds of the invention can be used in combination with chemotherapeutic agents, agonists or antagonists of nuclear receptors, or other anti-proliferative agents. The compounds of the invention can also be used in combination with a medical therapy such as surgery or radiotherapy, e.g., gamma-radiation, neutron beam radiotherapy, electron beam radiotherapy, proton therapy, brachytherapy, and systemic radioactive isotopes. Examples of suitable chemotherapeutic agents include any of: abarelix, aldesleukin, alemtuzumab, alitretinoin, allopurinol, all-trans retinoic acid, altretamine, anastrozole, arsenic trioxide, asparaginase, azacitidine, bendamustine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panobinostat, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinstat and zoledronate.

In some embodiments, the compounds of the invention can be used in combination with a therapeutic agent that targets an epigenetic regulator. Examples of epigenetic regulators include bromodomain inhibitors, the histone lysine methyltransferase inhibitors, histone arginine methyl transferase inhibitors, histone demethylase inhibitors, histone deacetylase inhibitors, histone acetylase inhibitors, and DNA methyltransferase inhibitors. Histone deacetylase inhibitors include, e.g., vorinostat. Histone arginine methyl transferase inhibitors include inhibitors of protein arginine methyltransferases (PRMTs) such as PRMT5, PRMT1 and PRMT4. DNA methyltransferase inhibitors include inhibitors of DNMT1 and DNMT3.

For treating cancer and other proliferative diseases, the compounds of the invention can be used in combination with targeted therapies, including JAK kinase inhibitors (e.g. Ruxolitinib), PI3 kinase inhibitors including PI3K-delta selective and broad spectrum PI3K inhibitors, MEK inhibitors, Cyclin Dependent kinase inhibitors, including CDK4/6 inhibitors and CDK9 inhibitors, BRAF inhibitors, mTOR inhibitors, proteasome inhibitors (e.g. Bortezomib, Carfilzomib), HDAC inhibitors (e.g. panobinostat, vorinostat), DNA methyl transferase inhibitors, dexamethasone, bromo and extra terminal family member (BET) inhibitors, BTK inhibitors (e.g. ibrutinib, acalabrutinib), BCL2 inhibitors (e.g. venetoclax), dual BCL2 family inhibitors (e.g. BCL2/BCLxL), PARP inhibitors, FLT3 inhibitors, or LSD1 inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), or PDR001. In some embodiments, the anti-PD-1 monoclonal antibody is nivolumab or pembrolizumab. In some embodiments, the anti-PD1 antibody is pembrolizumab. In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, durvalumab, or BMS-935559. In some embodiments, the inhibitor of an immune checkpoint molecule is an inhibitor of CTLA-4, e.g., an anti-CTLA-4 antibody. In some embodiments, the anti-CTLA-4 antibody is ipilimumab.

In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

Compounds of the present invention include, but are not limited to, those shown in Table 1.

TABLE 1

| Exam- | | |
|---|---|---|
| ple | | |
| # | Structure | Name |
| 1 | 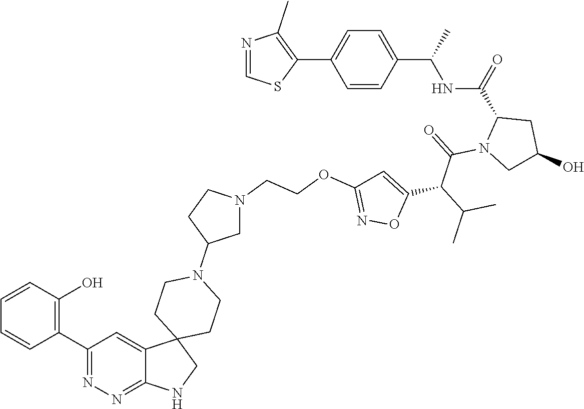 [SMILES: Cc1ncsc1-c1ccc([C@H](C)NC(=O)[C@@H]2C[C@@H](O)CN2C(=O)[C@@H](c2cc(OCCN3CCC(N4CCC5(CC4)CNc4nnc(-c6ccccc6O)cc45)C3)no2)C(C)C)cc1] | (2S,4R)-4-hydroxy-1- ((2R)-2-(3-(2-(3-(3′-(2- hydroxyphenyl)-6′,7′- dihydrospiro[piperidine- 4,5′-pyrrolo[2,3- c]pyridazin]-1- yl)pyrrolidin-1- yl)ethoxy)isoxazol-5- yl)-3-methylbutanoyl)- N-((S)-1-(4-(4- methylthiazol-5- yl)phenyl)ethyl) pyrrolidine-2- carboxamide |
| 2 | 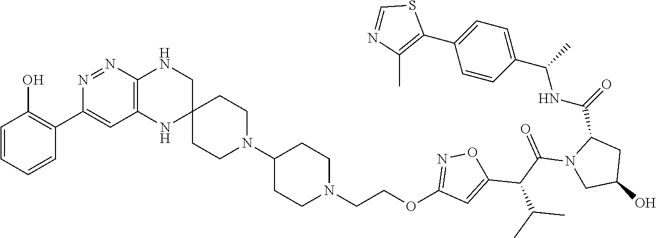 [SMILES: Cc1ncsc1-c1ccc([C@H](C)NC(=O)[C@@H]2C[C@@H](O)CN2C(=O)[C@@H](c2cc(OCCN3CCC(N4CCC5(CC4)CNc4nnc(-c6ccccc6O)cc4N5)CC3)no2)C(C)C)cc1] | (2S,4R)-4-hydroxy-1- ((R)-2-(3-(2-(4-(3′-(2- hydroxyphenyl)-7′,8′- dihydro-5′H- spiro[piperidine-4,6′- pyrazino[2,3- c]pyridazin]-1- yl)piperidin-1- yl)ethoxy)isoxazol-5- yl)-3-methylbutanoyl)- N-((S)-1-(4-(4- methylthiazol-5- yl)phenyl)ethyl) pyrrolidine-2- carboxamide |
| 3 | 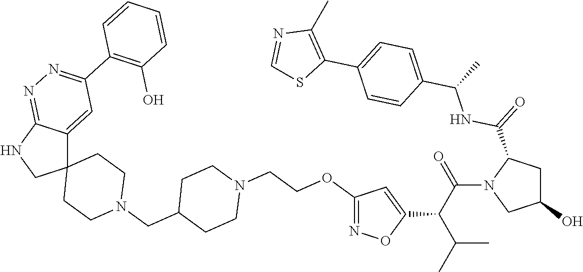 [SMILES: Cc1ncsc1-c1ccc([C@H](C)NC(=O)[C@@H]2C[C@@H](O)CN2C(=O)[C@@H](c2cc(OCCN3CCC(CN4CCC5(CC4)CNc4nnc(-c6ccccc6O)cc45)CC3)no2)C(C)C)cc1] | (2S,4R)-4-hydroxy-1- ((R)-2-(3-(2-(4-((3′-(2- hydroxyphenyl)-6′,7′- dihydrospiro[piperidine- 4,5′-pyrrolo[2,3- c]pyridazin]-1- yl)methyl)piperidin-1- yl)ethoxy)isoxazol-5- yl)-3-methylbutanoyl)- N-((S)-1-(4-(4- methylthiazol-5- yl)phenyl)ethyl) pyrrolidine-2- carboxamide |
| 4 | 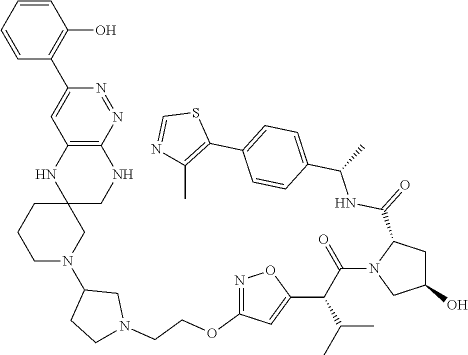 [SMILES: Cc1ncsc1-c1ccc([C@H](C)NC(=O)[C@@H]2C[C@@H](O)CN2C(=O)[C@@H](c2cc(OCCN3CCC(N4CCCC5(CNc6nnc(-c7ccccc7O)cc6N5)C4)C3)no2)C(C)C)cc1] | (2S,4R)-4-hydroxy-1- ((2R)-2-(3-(2-(3-(3′-(2- hydroxyphenyl)-7′,8′- dihydro-5′H- spiro[piperidine-3,6′- pyrazino[2,3- c]pyridazin]-1- yl)pyrrolidin-1- yl)ethoxy)isoxazol-5- yl)-3-methylbutanoyl)- N-((S)-1-(4-(4- methylthiazol-5- yl)phenyl)ethyl) pyrrolidine-2- carboxamide |
| 5 | 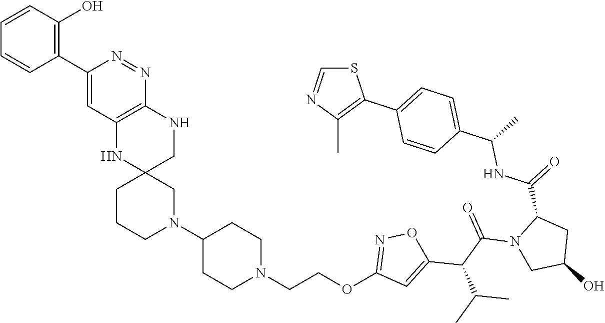 [SMILES: Cc1ncsc1-c1ccc([C@H](C)NC(=O)[C@@H]2C[C@@H](O)CN2C(=O)[C@@H](c2cc(OCCN3CCC(N4CCCC5(CNc6nnc(-c7ccccc7O)cc6N5)C4)CC3)no2)C(C)C)cc1] | (2S,4R)-4-hydroxy-1- ((2R)-2-(3-(2-(4-(3′-(2- hydroxyphenyl)-7′,8′- dihydro-5′H- spiro[piperidine-3,6′- pyrazino[2,3- c]pyridazin]-1- yl)piperidin-1- yl)ethoxy)isoxazol-5- yl)-3-methylbutanoyl)- N-((S)-1-(4-(4- methylthiazol-5- yl)phenyl)ethyl) pyrrolidine-2- carboxamide |
| 6 | 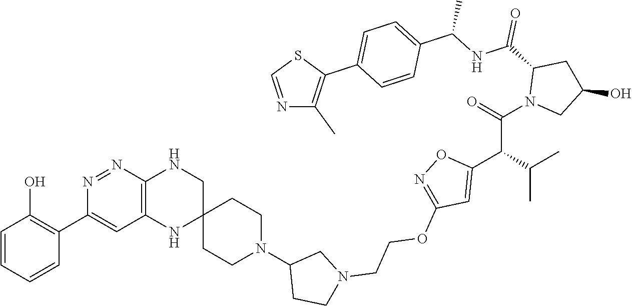 [SMILES: Cc1ncsc1-c1ccc([C@H](C)NC(=O)[C@@H]2C[C@@H](O)CN2C(=O)[C@@H](c2cc(OCCN3CCC(N4CCC5(CC4)CNc4nnc(-c6ccccc6O)cc4N5)C3)no2)C(C)C)cc1] | (2S,4R)-4-hydroxy-1- ((2R)-2-(3-(2-(3-(3′-(2- hydroxyphenyl)-7′,8′- dihydro-5′H-spiro [piperidine-4,6′- pyrazino[2,3- c]pyridazin]-1- yl)pyrrolidin-1- yl)ethoxy)isoxazol-5- yl)-3-methylbutanoyl)- N-((S)-1-(4-(4- methylthiazol-5- yl)phenyl)ethyl) pyrrolidine-2- carboxamide |
| 7 | 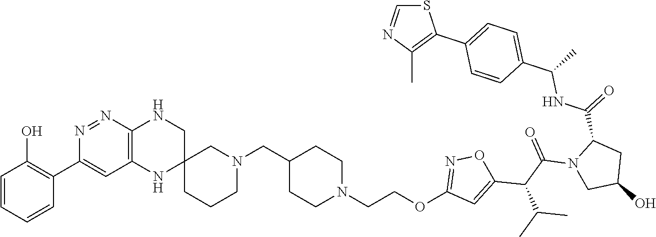 [SMILES: Cc1ncsc1-c1ccc([C@H](C)NC(=O)[C@@H]2C[C@@H](O)CN2C(=O)[C@@H](c2cc(OCCN3CCC(CN4CCCC5(CNc6nnc(-c7ccccc7O)cc6N5)C4)CC3)no2)C(C)C)cc1] | (2S,4R)-4-hydroxy-1- ((2R)-2-(3-(2-(4-((3′-(2- hydroxyphenyl)-7′,8′- dihydro-5′H- spiro[piperidine-3,6′- pyrazino[2,3- c]pyridazin]-1- yl)methyl)piperidin-1- yl)ethoxy)isoxazol-5- yl)-3-methylbutanoyl)- N-((S)-1-(4-(4- methylthiazol-5- yl)phenyl)ethyl) pyrrolidine-2- carboxamide |
| 8 | 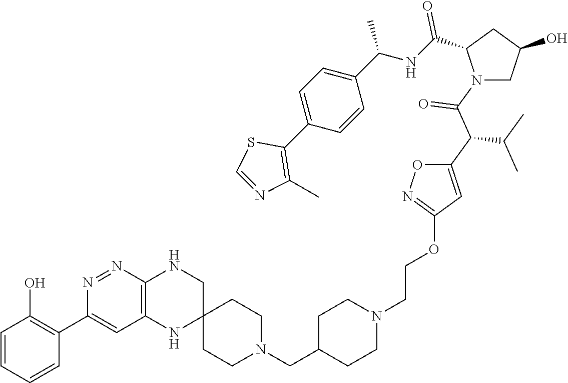 [SMILES: Cc1ncsc1-c1ccc([C@H](C)NC(=O)[C@@H]2C[C@@H](O)CN2C(=O)[C@@H](c2cc(OCCN3CCC(CN4CCC5(CC4)CNc4nnc(-c6ccccc6O)cc4N5)CC3)no2)C(C)C)cc1] | (2S,4R)-4-hydroxy-1- ((R)-2-(3-(2-(4-((3′-(2- hydroxyphenyl)-7′,8′- dihydro-5′H-spiro [piperidine-4,6′- pyrazino[2,3- c]pyridazin]-1- yl)methyl)piperidin-1- yl)ethoxy)isoxazol-5- yl)-3-methylbutanoyl)- N-((S)-1-(4-(4- methylthiazol-5- yl)phenyl)ethyl) pyrrolidine-2- carboxamide |

### EXAMPLES

The compounds of the invention may be prepared using the general procedures, schemes and intermediates described below.

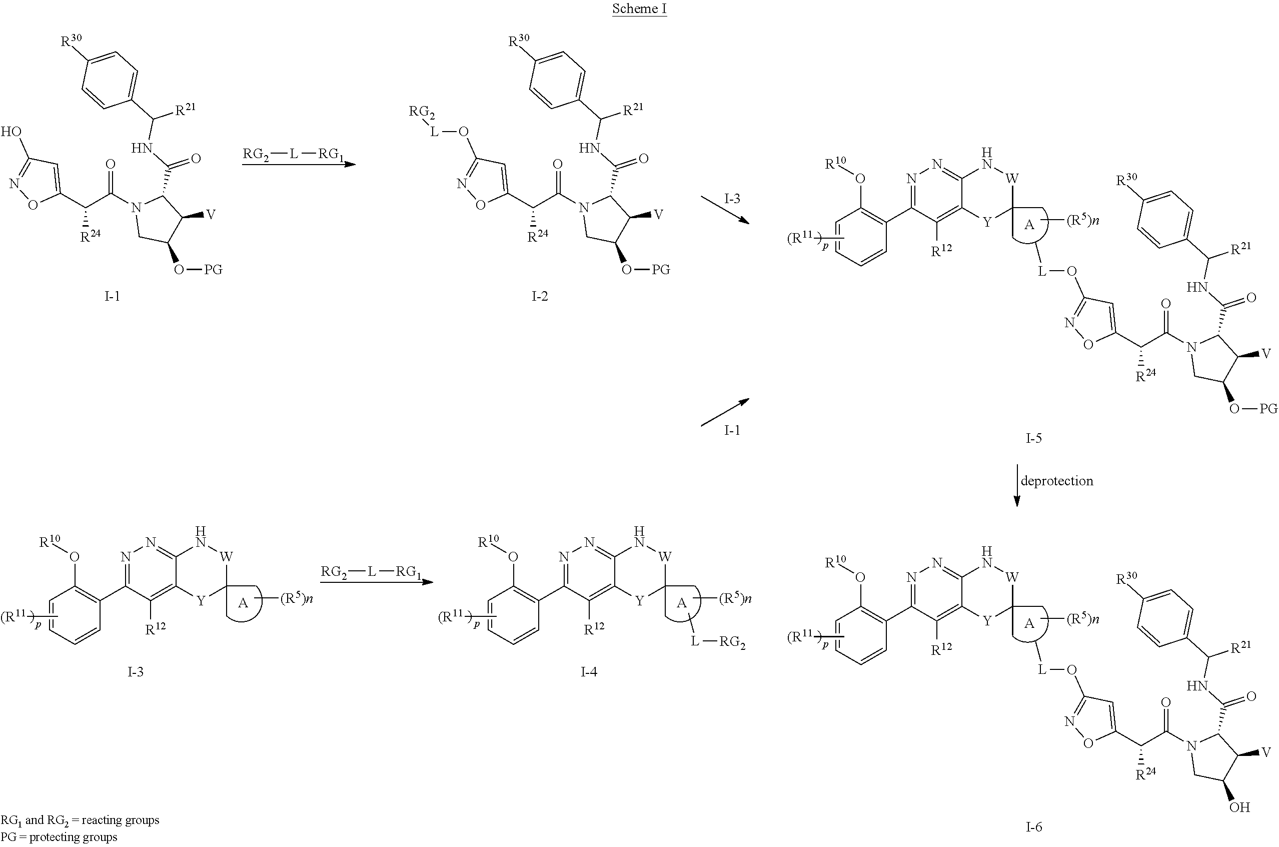

The compounds of formula I-6 can be synthesized using, for example, the sequences shown in Scheme I. Coupling of compounds I-1 with RG₁ using appropriate synthetic methods (such as but not limited to SN2 reaction, SNAr reaction, Mitsunobu reaction, etc.) can afford compounds I-2. Compounds I-3 can be introduced using appropriate synthetic methods (such as, but not limited to, SN2 reaction, SNAr reaction, reductive amination, Buchwald reaction, amide formation, Mitsunobu reaction, olefin metathesis, etc.) to give compounds I-5. Alternatively, the synthesis of I-4 can be achieved by the coupling of I-3 with RG₁, followed by the introduction of intermediates I-1 using appropriate synthetic methods mentioned above. Removal of the protecting groups can result in compounds of formula I-6.

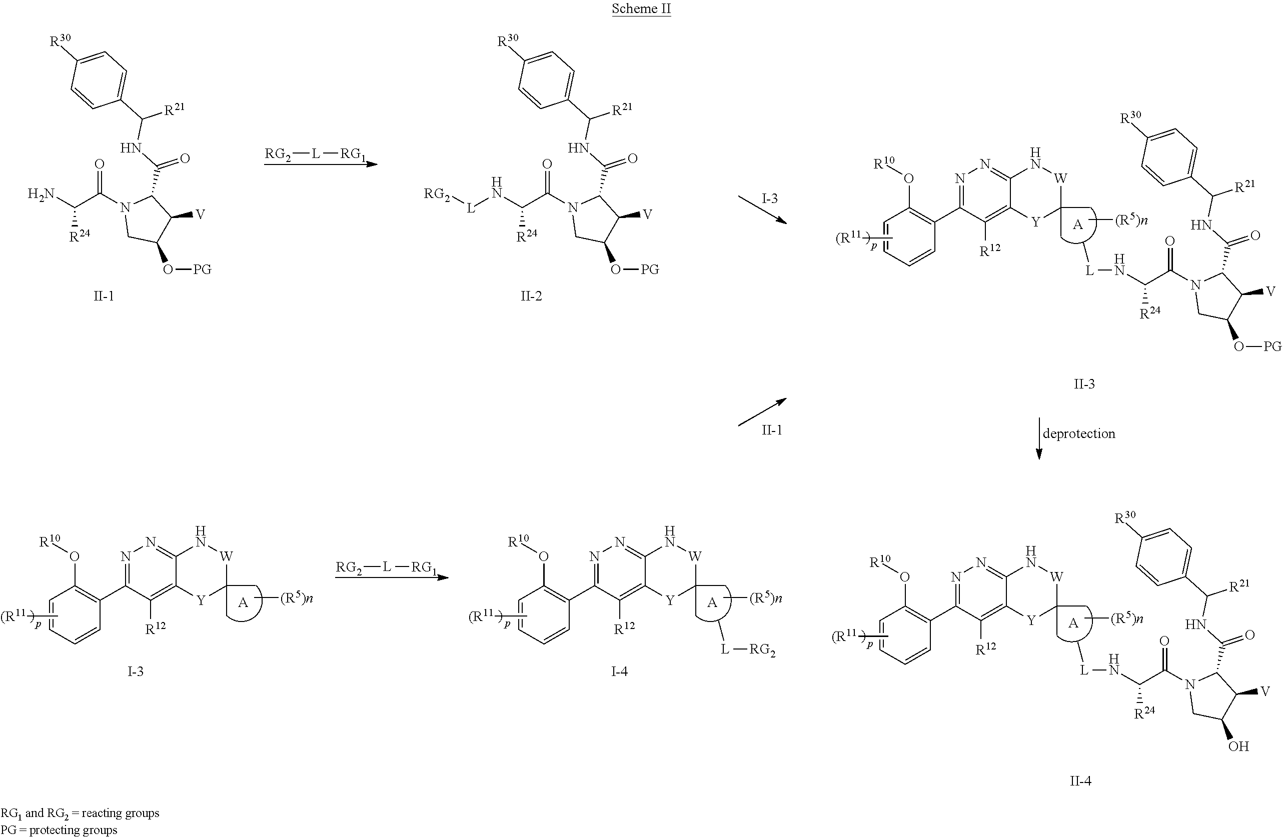

Compounds of formula II-4 can be synthesized using, for example, the sequences shown in Scheme II. Coupling of compounds II-1 with RG₁ using appropriate synthetic methods (such as but not limited to amide formation, SN2 reaction, reductive amination, etc.; e.g., RG₁ is a leaving group, such as a bromide and is displaced the amine of II-1) can afford compounds II-2.

Compounds I-3 can be introduced using appropriate synthetic methods (such as, but not limited to, SN2 reaction, SNAr reaction, reductive amination, Buchwald reaction, amide formation, Mitsunobu reaction, olefin metathesis, etc.) to give compounds II-3. Alternatively, the synthesis of II-3 can be achieved by the coupling of I-3 with RG₁, followed by the introduction of II-1 using appropriate synthetic methods mentioned above. Removal of the protecting groups can afford compounds of formula II-4.

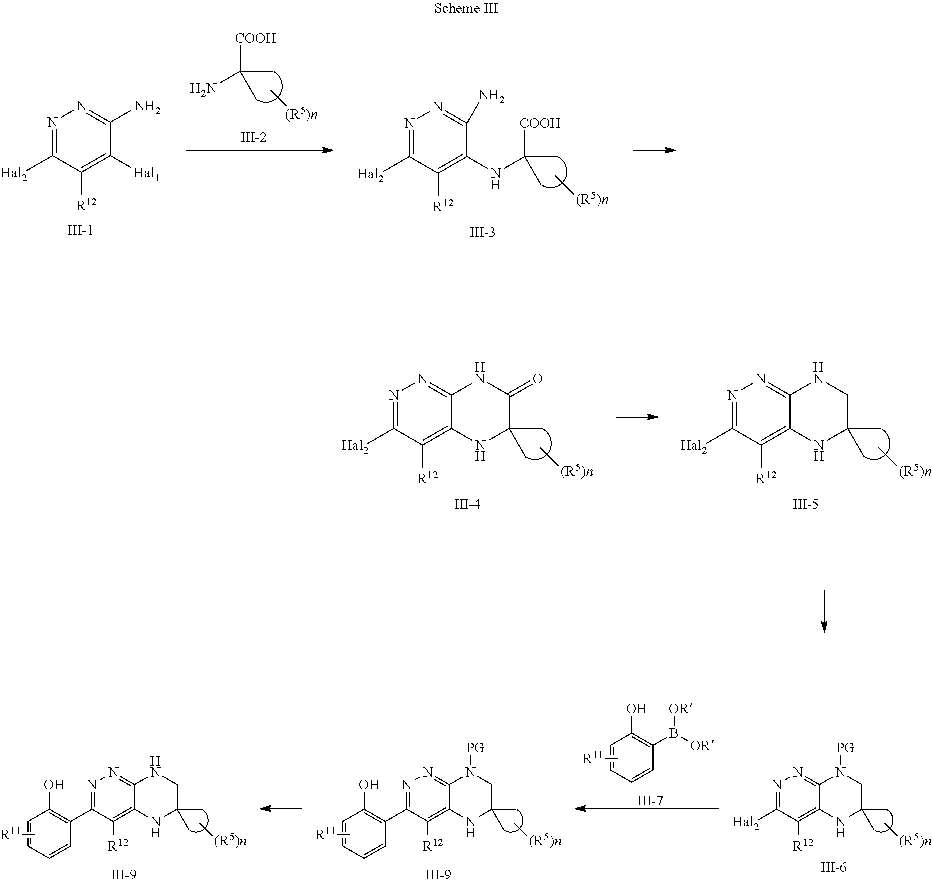

Compounds of formula III-9 can be synthesized using, for example, the sequences shown in Scheme III. The coupling between III-1 and amino acid III-2 under standard Ullmann conditions (e.g., in the presence of a Cu catalyst, such as CuI, and a base, such as K₂CO₃) can give compound III-3. The cyclization under amide formation conditions (e.g. treatment with an appropriate base such as DIPEA or trimethylanine and in the presence of coupling agents such as HATU, HOBt, or PyBOP) can afford compound III-4. The reduction of carbonyl group using BH₃ or LAI can provide compound III-5. After protection of the NH groups and the coupling between III-6 and III-7 under standard Suzuki conditions (e.g. in the presence of a palladium catalyst, such as tetrakis(triphenylphosphine)palladium(0) or [1,1′-bis(diphenylphosphino) ferrocene]dichloropalladium (II), complex with dichloromethane and a base (e.g., a carbonate base) can produce compound III-8. Removal of the protecting groups can afford compounds of formula III-9.

### Intermediate 1: 2-(6′,7′-dihydrospiro[piperidine-4,5′-pyrrolo[2,3-c]pyridazin]-3′-yl)phenol (Int-1)

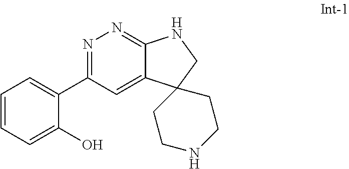 [SMILES: Oc1ccccc1-c1cc2c(nn1)NCC21CCNCC1]

### Step 1: 3,6-dichloro-N-(2,4-dimethoxybenzyl)pyridazin-4-amine

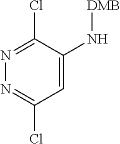

A solution of 2,4-dimethoxybenzylamine (150 g, 900 mmol, 1.1 eq) and 3,4,6-trichloropyridazine (150 g, 818 mmol, 1.0 eq) in i-PrOH (300 mL) was stirred at 75° C. for 18 h. The reaction mixture was concentrated and the residue was purified by column chromatography on a silica gel column (PE/EA=2/1) to afford 3,6-dichloro-N-[(2,4-dimethoxyphenyl)methyl]pyridazin-4-amine (184 g, 584 mmol, 71.5% yield) as a white solid. LCMS calculated for C₁₃H₁₄Cl₂N₃O₂ (M+H)⁺ m/z=314.0/316.0; found: 314.0/316.0.

### Step 2: benzyl 3′-chloro-7′-(2,4-dimethoxybenzyl)-6′-oxo-6′,7′-dihydrospiro[piperidine-4,5′-pyrrolo[2,3-c]pyridazine]-1-carboxylate

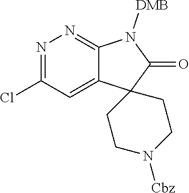

A solution of benzyl 4-carbonochloridoylpiperidine-1-carboxylate (117 g, 414 mmol, 2.6 eq), TEA (80.5 g, 796 mmol, 5.0 eq) and 3,6-dichloro-N-[(2,4-dimethoxyphenyl)methyl]pyridazin-4-amine (50.0 g, 159 mmol, 1.0 eq) in DCM (300 mL) was stirred at 45° C. for 3 days. The reaction mixture was diluted with water and extracted with EtOAc (250 mL×2). The combined organic layers were washed with sat. aqueous NaHCO₃ and brine, dried over anhydrous Na₂SO₄, and filtered. The filtrate was concentrated under reduced pressure. The residue and Cs₂CO₃ (51.9 g, 159 mmol, 1.0 eq) in DMF (100 mL) was stirred at 80° C. for 2 h. The volatiles were removed and the residue was purified by column chromatography on a silica gel column (PE/EA=2/1) to afford benzyl 3′-chloro-7′-[(2,4-dimethoxyphenyl)methyl]-6′-oxospiro[piperidine-4,5′-pyrrolo[2,3-c]pyridazine]-1-carboxylate (64.0 g, 122 mmol, 76.9% yield) as a yellow oil. LCMS calculated for C₂₇H₂₈ClN₄O₅ (M+H)⁺ m/z=523.2; found: 523.3.

### Step 3: benzyl 7′-(2,4-dimethoxybenzyl)-3′-(2-hydroxyphenyl)-6′-oxo-6′,7′-dihydrospiro[piperidine-4,5′-pyrrolo[2,3-c]pyridazine]-1-carboxylate (GXL008-11-1)

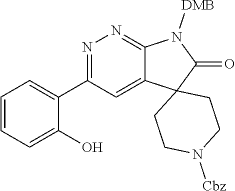

A mixture of benzyl 3′-chloro-7′-[(2,4-dimethoxyphenyl)methyl]-6′-oxospiro [piperidine-4,5′-pyrrolo[2,3-c]pyridazine]-1-carboxylate (64.0 g, 122 mmol, 1.0 eq), 2-hydroxyphenylboronic acid (33.8 g, 245 mmol, 2.0 eq), Na₂CO₃ (45.4 g, 428 mmol, 3.5 eq) and Pd(dppf)Cl₂ (7.98 g, 12.2 mmol, 0.1 eq) in toluene (500 mL) and water (100 mL) was stirred at 105° C. under N₂ for 18 h. The reaction mixture was diluted with water and extracted with EtOAc (250 mL×2). The combined organic layers were washed with brine, dried over anhydrous Na₂SO₄, and filtered. The filtrate was concentrated and the residue was purified by column chromatography on a silica gel column (PE/EA=2/1) to afford benzyl 7′-[(2,4-dimethoxyphenyl)methyl]-3′-(2-hydroxyphenyl)-6′-oxospiro[piperidine-4,5′-pyrrolo[2,3-c]pyridazine]-1-carboxylate (42.0 g, 72.3 mmol, 59.1% yield) as a white solid. LCMS calculated for C₃₂H₃₁N₄O₅ (M+H)⁺ m/z=581.2; found: 581.2.

### Step 4: benzyl 7′-(2,4-dimethoxybenzyl)-3′-(2-hydroxyphenyl)-6′,7′-dihydrospiro [piperidine-4,5′-pyrrolo[2,3-c]pyridazine]-1-carboxylate

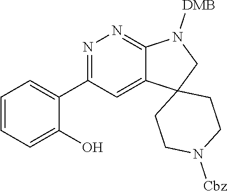

To the mixture of TiCl₄ (217 mL, 217 mmol, 3.0 eq) in DME (1.13 L) was added NaBH₄ (169 g, 434 mmol, 6.0 eq) in portions at 5° C. The mixture was stirred at this temperature for 1 h. Then benzyl 7′-[(2,4-dimethoxyphenyl)methyl]-3′-(2-hydroxyphenyl)-6′-oxospiro[piperidine-4,5′-pyrrolo[2,3-c]pyridazine]-1-carboxylate (42.0 g, 72.3 mmol, 1.0 eq) was slowly added and the mixture was stirred at 5° C. under N₂ for 2 h. The reaction mixture was quenched with ice/water and basified with NH₃·H₂O. The organic phase was separated, and the aqueous phase was extracted with EA (500 mL×2). The combined organic layers were washed with brine, dried over anhydrous Na₂SO₄, and filtered. The filtrate was concentrated and the residue was purified by column chromatography on a silica gel column (PE/EA=2/1) to afford benzyl 7-[(2,4-dimethoxyphenyl)methyl]-3-(2-hydroxyphenyl)spiro[6H-pyrrolo[2,3-c]pyridazine-5,4′-piperidine]-1′-carboxylate (8.20 g, 14.5 mmol, 20.0% yield) as a white solid. LCMS calculated for C₃₃H₃₅N₄O₅ (M+H)⁺ m/z=567.3; found: 567.2.

### Step 5: 2-(6′,7′-dihydrospiro[piperidine-4,5′-pyrrolo[2,3-c]pyridazin]-3′-yl)phenol (GXL008-43-1)

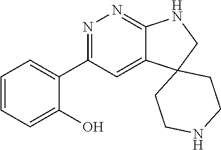 [SMILES: Oc1ccccc1-c1cc2c(nn1)NCC21CCNCC1]

To the mixture of benzyl 7-[(2,4-dimethoxyphenyl)methyl]-3-(2-hydroxyphenyl) spiro[6H-pyrrolo[2,3-c]pyridazine-5,4′-piperidine]-1′-carboxylate (8.20 g, 14.5 mmol, 1.0 eq) in TfOH (12.8 mL, 145 mmol, 10 eq) was added Tf₂O (4.31 mL, 58.0 mmol, 4.0 eq) at 5° C. under N₂. Then the reaction was stirred at room temperature for 3 h. The reaction mixture was quenched with ice/water. The precipitate was removed by filtration and the filtrate was concentrated and purified by prep-HPLC on a C₁₈ column (20-35 uM, 100 A, 330 g) with mobile phase: H₂O (0.1% TFA)/MeOH at flow rate: 50 mL/min to afford 2-spiro[6,7-dihydropyrrolo[2,3-c]pyridazine-5,4′-piperidine]-3-ylphenol (5.80 g, 14.1 mmol, 97.4% yield) as a TFA salt. LCMS calculated for C₁₆H₁₉N₄O (M+H)⁺ m/z=283.3; found: 283.2. ¹H NMR (400 MHz, CD₃OD) δ 8.10 (s, 1H), 7.64 (dd, J=8.0 Hz, 1.6 Hz, 1H), 7.32-7.39 (m, 1H), 6.99-7.02 (m, 2H), 3.94 (s, 2H), 3.48-3.52 (m, 2H), 3.22-3.31 (m, 2H), 2.12-2.22 (m, 4H).

### Intermediate 2: 2-(7′,8′-dihydro-5′H-spiro[piperidine-4,6′-pyrazino[2,3-c]pyridazin]-3′-yl)phenol (Int-2)

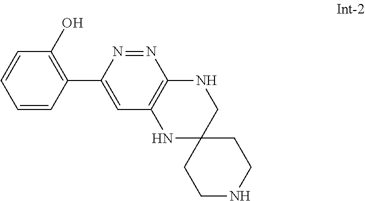 [SMILES: Oc1ccccc1-c1cc2c(nn1)NCC1(CCNCC1)N2]

### Step 1: 4-((3-amino-6-chloropyridazin-4-yl)amino)-1-((benzyloxy)carbonyl)piperidine-4-carboxylic acid

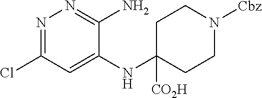 [SMILES: Nc1nnc(Cl)cc1NC1(C(=O)O)CCN(C(=O)OCc2ccccc2)CC1]

To a solution of Copper(I) bromide-dimethyl sulfide (37.9 mg, 0.20 mmol) in DMA (5 mL) was added 4-amino-1-phenylmethoxycarbonylpiperidine-4-carboxylic acid (1.0 g, 3.6 mmol), K₂CO₃ (207 mg, 1.5 mmol), 4-bromo-6-chloropyridazin-3-amine (748 mg, 3.6 mmol). The mixture was stirred at 120° C. for 3 h. The reaction mixture was diluted with EtOAc (100 mL) and washed with brine (30 mL×2). The organic layer was concentrated in vacuo and the residue was purified by prep-HPLC to give 4-((3-amino-6-chloropyridazin-4-yl)amino)-1-((benzyloxy)carbonyl)piperidine-4-carboxylic acid (330 mg, 0.81 mmol, 22.6% yield) as a yellow solid. LCMS calculated for C₁₈H₂₁ClN₅O₄ (M+H)⁺ m/z=406.1; found: 406.2.

### Step 2: benzyl 3′-chloro-7′-oxo-7′,8′-dihydro-5′H-spiro[piperidine-4,6′-pyrazino[2,3-c]pyridazine]-1-carboxylate

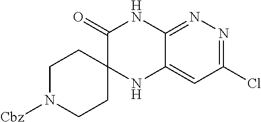 [SMILES: O=C(OCc1ccccc1)N1CCC2(CC1)Nc1cc(Cl)nnc1NC2=O]

To a stirred solution of 4-((3-amino-6-chloropyridazin-4-yl)amino)-1-((benzyloxy) carbonyl)piperidine-4-carboxylic acid (300 mg, 0.74 mmol) in DMF (2 mL) was added DIEA (0.37 mL, 2.22 mmol) and HATU (562 mg, 1.48 mmol) at 25° C. After for 2 h, the reaction mixture was diluted with H₂O and extracted with EtOAc (10 mL×3). The organic layers were combined and the volatiles were removed in vacuo. The residue was purified by silica column gel chromatography to give benzyl 3′-chloro-7′-oxo-7′,8′-dihydro-5′H-spiro[piperidine-4,6′-pyrazino[2,3-c]pyridazine]-1-carboxylate (300 mg, 0.77 mmol, 100% yield) as a yellow oil. LCMS calculated for C₁₈H₁₉ClN₅O₃ (M+H)⁺ m/z=388.1; found: 388.3.

### Step 3: benzyl 3′-chloro-7′,8′-dihydro-5′H-spiro[piperidine-4,6′-pyrazino[2,3-c]pyridazine]-I-carboxylate

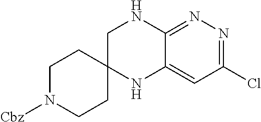 [SMILES: O=C(OCc1ccccc1)N1CCC2(CC1)CNc1nnc(Cl)cc1N2]

To a solution of benzyl 3′-chloro-7′-oxo-7′,8′-dihydro-5′H-spiro[piperidine-4,6′-pyrazino[2,3-c]pyridazine]-1-carboxylate (300 mg, 0.77 mmol) in THF (20 mL) was added borane (1M in THF, 2.3 mL, 2.3 mmol). The mixture was stirred at 65° C. for 16 h. The volatiles were removed under reduced pressure. The residue was dissolved in MeOH (20 mL) and stirred at 65° C. for additional 4 h. The reaction mixture was concentrated in vacuo to afford crude benzyl 3′-chloro-7′,8′-dihydro-5′H-spiro[piperidine-4,6′-pyrazino[2,3-c]pyridazine]-1-carboxylate (300 mg, 0.80 mmol, 100% yield) as a yellow oil, which was used in the next step without further purification. LCMS calculated for C₁₈H₂₁ClN₅O₂ (M+H)⁺ m/z=374.1; found: 374.3.

### Step 4: 1-benzyl 5′,8′-di-tert-butyl 3′-chloro-5′H-spiro[piperidine-4,6′-pyrazino[2,3-c]pyridazine]-1,5′,8′(7′H)-tricarboxylate

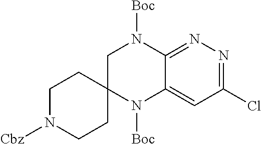 [SMILES: CC(C)(C)OC(=O)N1CC2(CCN(C(=O)OCc3ccccc3)CC2)N(C(=O)OC(C)(C)C)c2cc(Cl)nnc21]

A solution of di-tert butyl dicarbonate (350 mg, 1.6 mmol), benzyl 3-chlorospiro[7,8-dihydro -5H-pyrazino[2,3-c]pyridazine-6,4′-piperidine]-1′-carboxylate (300 mg, 0.80 mmol), TEA (0.22 mL, 1.6 mmol) and 4-(dimethylamino)pyridine (98 mg, 0.80 mmol) in DCM (10 mL) was stirred at 25° C. for 16 h. The volatiles were removed in vacuo and the residue was purified by prep-TLC to get the desired product (130 mg, 0.22 mmol, 28.2% yield). LCMS calculated for C₂₈H₃₇ClN₅O₆ (M+H)⁺ m/z=574.2; found: 574.3.

### Step 5: 1-benzyl 5′,8′-di-tert-butyl 3′-(2-hydroxyphenyl)-5′H-spiro[piperidine-4,6′-pyrazino[2,3-c]pyridazine]-1,5′,8′(7′H)-tricarboxylate

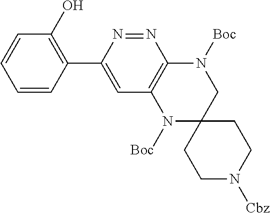 [SMILES: CC(C)(C)OC(=O)N1CC2(CCN(C(=O)OCc3ccccc3)CC2)N(C(=O)OC(C)(C)C)c2cc(-c3ccccc3O)nnc21]

The mixture of benzyl 3′-chloro-7′,8′-dihydro-5′H-spiro[piperidine-4,6′-pyrazino[2,3-c]pyridazine]-1-carboxylate (133 mg, 0.23 mmol), potassium carbonate (321 mg, 2.33 mmol), 1,1′-bis(diphenylphosphino)ferrocene-palladium(II)dichloride dichloromethane complex (9.5 mg, 0.01 mmol) and 2-hydroxyphenylboronic acid (160 mg, 1.16 mmol) in 1,4-dioxane (10 mL) and water (2 mL) was stirred at 110° C. for 16 h under the atmosphere of N₂. The volatiles were removed in vacuo and the residue was purified by prep-HPLC, eluted with MeCN in water, 0.10% TFA, from 10% to 90% to afford 1-benzyl 5′,8′-di-tert-butyl 3′-(2-hydroxyphenyl)-5′H-spiro[piperidine-4,6′-pyrazino[2,3-c]pyridazine]-1,5′,8′(7′H)-tricarboxylate (40 mg, 0.06 mmol, 27.2% yield) and as a yellow oil. LCMS calculated for C₃₄H₄₂N₅O₇ (M+H)⁺ m/z=632.3; found: 632.3.

### Step 6: di-tert-butyl 3′-(2-hydroxyphenyl)-5′H-spiro[piperidine-4,6′-pyrazino[2,3-c]pyridazine]-5′,8′(7′H)-dicarboxylate

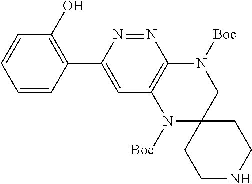 [SMILES: CC(C)(C)OC(=O)N1CC2(CCNCC2)N(C(=O)OC(C)(C)C)c2cc(-c3ccccc3O)nnc21]

1-benzyl 5′,8′-di-tert-butyl 3′-(2-hydroxyphenyl)-5′H-spiro[piperidine-4,6′-pyrazino[2,3-c]pyridazine]-1,5′,8′(7′H)-tricarboxylate (43 mg, 0.07 mmol) and Pd/C (7.42 mg, 0.07 mmol) was added to methanol (10 mL) and stirred at 25° C. for 1 h under the atmosphere of H₂. The reaction mixture was filtered and the filtrate was concentrated to afford the crude product (30 mg, 0.06 mmol, 86.5% yield) and as a yellow oil. LCMS calculated for C₂₆H₃₆N₅O₅ (M+H)⁺ m/z=498.3; found: 498.3.

### Step 7: 2-(7′,8′-dihydro-5′H-spiro[piperidine-4,6′-pyrazino[2,3-c]pyridazin]-3′-yl)phenol (Int-2)

To a stirred solution of di-tert-butyl 3′-(2-hydroxyphenyl)-5′H-spiro[piperidine-4,6′-pyrazino[2,3-c]pyridazine]-5′,8′(7′H)-dicarboxylate (30 mg, 0.06 mmol) in DCM (3 mL) was added TFA (1.0 mL) at rt. After 1 h, the volatiles were removed and the residue was purified by prep-HPLC, eluted with CH₃CN in H₂O (0.1% TFA) from 10% to 90% to get 2-(7′,8′-dihydro-5′H-spiro[piperidine-4,6′-pyrazino[2,3-c]pyridazin]-3′-yl)phenol as its TFA salt (7.2 mg, 0.01 mmol, 21.8% yield). LCMS calculated for C₁₆H₂₀N₅O (M+H)⁺ m/z=298.2; found: 298.2. ¹HNMR (CD₃OD-d₄, 400 MHz): δ 7.45-7.41 (m, 2H), 7.10 (s, 1H), 7.04-7.00 (m, 2H), 3.45-3.30 (m, 6H), 2.07-2.04 (m, 4H).

### Intermediate 3: 2-(7′,8′-dihydro-5′H-spiro[piperidine-3,6′-pyrazino[2,3-c]pyridazin]-3′-yl)phenol

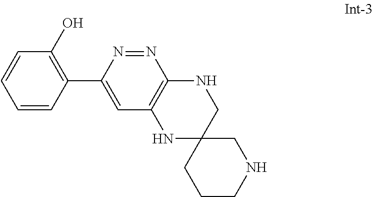 [SMILES: Oc1ccccc1-c1cc2c(nn1)NCC1(CCCNC1)N2]

The title compound was prepared using procedure analogous to those described for Int-2, using appropriate starting materials. LCMS calculated for C₁₆H₂₀N₅O (M+H)⁺ m/z=298.2; found: 298.3. 1HNMR (CD₃OD-d₄, 400 MHz): δ 7.60-7.62 (m, 1H), 7.18-7.23 (m, 1H), 7.05 (s, 1H), 6.83-6.88 (m, 2H), 3.47-3.50 (m, 1H), 3.19-3.22 (m, 1H), 2.64-2.91 (m, 4H), 1.62-1.77 (m, 4H).

### Intermediate 4: (2S,4R)-4-((tert-butyldimethylsilyl)oxy)-1-((R)-2-(3-hydroxyisoxazol-5-yl)-3-methylbutanoyl)-N—((S)-1-(4-(4-methylthiazol-5-yl)phenyl)ethyl)pyrrolidine-2-carboxamide

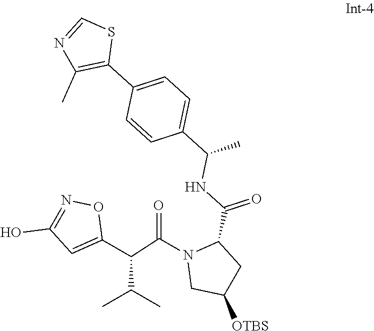 [SMILES: Cc1ncsc1-c1ccc([C@H](C)NC(=O)[C@@H]2C[C@@H](O[Si](C)(C)C(C)(C)C)CN2C(=O)[C@@H](c2cc(O)no2)C(C)C)cc1]

23 g of (2S,4R)-4-((tert-butyldimethylsilyl)oxy)-1-(2-(3-hydroxyisoxazol-5-yl)-3-methylbutanoyl)-N—((S)-1-(4-(4-methylthiazol-5-yl)phenyl)ethyl)pyrrolidine-2-carboxamide (prepared using the procedure described in US2020/0038378) was purified on chiral supercritical fluid chromatography using the following conditions: column: DAICEL CHIRALPAK® Whelk (250*25 mm 10 mm); mobile phase: 50% IPA/CO₂; pressure: 100 bar; flow rate: 70 g/min; UV: 214 nM; injection: 2.5 mL, 76.7 mg/mL in MeOH. 10.7 g (peak 1, t, =5.0 min) (2S,4R)-4-((tert-butyldimethylsilyl)oxy)-1-((R)-2-(3-hydroxyisoxazol-5-yl)-3-methylbutanoyl)-N—((S)-1-(4-(4-methylthiazol-5-yl)phenyl)ethyl)pyrrolidine-2-carboxamide was afforded. LCMS m/z calcd for C₃₁H₄₅N₄O₅SSi [M+H]⁺: 613.3; Found: 613.3.

### Intermediate 5: (2S,4R)-1-((R)-2-(3-(2-bromoethoxy)isoxazol-5-yl)-3-methylbutanoyl)-4-((tert-butyldimethylsilyl)oxy)-N—((S)-1-(4-(4-methylthiazol-5-yl)phenyl)ethyl)pyrrolidine-2-carboxamide (Int-5)

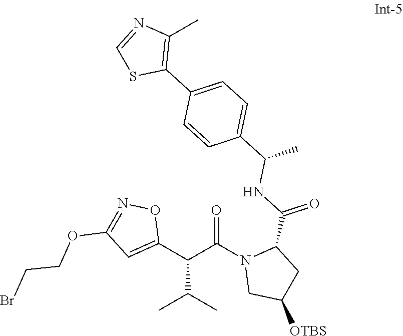 [SMILES: Cc1ncsc1-c1ccc([C@H](C)NC(=O)[C@@H]2C[C@@H](O[Si](C)(C)C(C)(C)C)CN2C(=O)[C@@H](c2cc(OCCBr)no2)C(C)C)cc1]

To a solution of (2S,4R)-4-((tert-butyldimethylsilyl)oxy)-1-((R)-2-(3-hydroxyisoxazol-5-yl)-3-methylbutanoyl)-N—((S)-1-(4-(4-methylthiazol-5-yl)phenyl)ethyl)pyrrolidine-2-carboxamide (100 mg, 0.16 mmol) and 1,2-dibromoethane (45 mg, 0.24 mmol) in DMF (3 mL) was added and NaHCO₃ (60 mg, 0.67 mmol) at 25° C., the mixture was stirred at 30° C. for 16 h. The reaction was taken up in EtOAc (20 mL) and the organics were washed with water (20 mL×3) and brine (20 mL). The organics were then separated and dried (MgSO₄) before concentration to dryness. The crude product was then purified by prep-TLC (EtOAc:PE=2:1) to give (2S,4R)-1-((R)-2-(3-(2-bromoethoxy)isoxazol-5-yl)-3-methylbutanoyl)-4-((tert-butyldimethylsilyl)oxy)-N—((S)-1-(4-(4-methylthiazol-5-yl)phenyl)ethyl)pyrrolidine-2-carboxamide (50 mg, 0.07 mmol, 51% yield) as a sticky colorless solid. LCMS calc'd for C₃₃H₄₇BrN₄O₅SSi: 718.2; Found: LCMS [M+H]: 719.3.

### Intermediate 6: 2-(1-(pyrrolidin-3-yl)-6′,7′-dihydrospiro[piperidine-4,5′-pyrrolo[2,3-c]pyridazin]-3′-yl)phenol

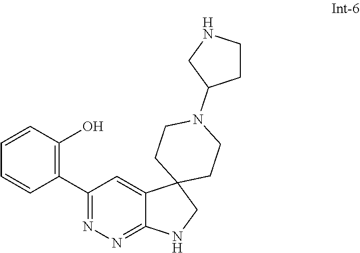 [SMILES: Oc1ccccc1-c1cc2c(nn1)NCC21CCN(C2CCNC2)CC1]

### Step 1: tert-butyl 3-(3′-(2-hydroxyphenyl)-6′,7′-dihydrospiro[piperidine-4,5′-pyrrolo[2,3-c]pyridazin]-1-yl)pyrrolidine-1-carboxylate

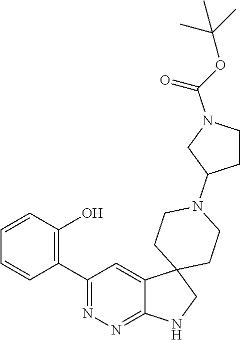 [SMILES: CC(C)(C)OC(=O)N1CCC(N2CCC3(CC2)CNc2nnc(-c4ccccc4O)cc23)C1]

A mixture of boc-3-pyrrolidinone (59 mg, 0.32 mmol) and 2-(6′,7′-dihydrospiro [piperidine-4,5′-pyrrolo[2,3-c]pyridazin]-3′-yl)phenol (45 mg, 0.16 mmol) in methanol (10 mL) was added sodium cyanoborohydride (30 mg, 0.48 mmol). The reaction was stirred at 25° C. for 16 h. The reaction was diluted with H₂O and extracted with EA. The organic layers were combined, dried over Na₂SO₄, filtered, and concentrated to give the crude product as a yellow oil (30 mg, 0.06 mmol, 41.6% yield). LCMS m/z calcd for C₂₅H₃₄N₅O₃ [M+H]⁺: 452.3; found: 452.3.

### Step 2: 2-(1-(pyrrolidin-3-yl)-6′,7′-dihydrospiro[piperidine-4,5′-pyrrolo[2,3-c]pyridazin]-3′-yl)phenol

A mixture of TFA (0.5 mL) and tert-butyl 3-(3′-(2-hydroxyphenyl)-6′,7′-dihydrospiro [piperidine-4,5′-pyrrolo[2,3-c]pyridazin]-1-yl)pyrrolidine-1-carboxylate (35 mg, 0.08 mmol) in DCM (5 mL) was stirred at 25° C. for 1 h. The reaction was quenched with saturated aqueous NaHCO₃ and extracted with DCM/MeOH (10/1 v/v). The organic layers were combined, dried over Na₂SO₄, filtered, and concentrated to give the crude product as a yellow oil (35 mg, 0.09 mmol, 100% yield) as a yellow oil. LCMS m/z calcd for C₂₀H₂₆N₅O [M+H]⁺: 352.3; found: 352.3.

### Intermediates 7-14

The intermediates shown below in Table 2 were prepared by the method used in preparing Intermediate 6 (Int-6) using appropriate starting materials.

TABLE 2

| | | | Calcd. | Found |
|---|---|---|---|---|
| | | | (M + H)⁺ | (M + H)⁺ |
| Int. | Structure | Name | m/z | m/z |
| Int-7 | 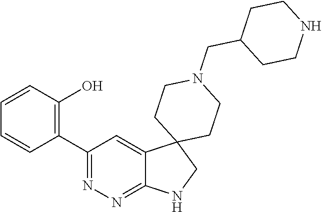 [SMILES: Oc1ccccc1-c1cc2c(nn1)NCC21CCN(CC2CCNCC2)CC1] | 2-(1-(piperidin-4- ylmethyl)-6′,7′- dihydrospiro[piperidine- 4,5′-pyrrolo[2,3- c]pyridazin]-3′-yl) phenol | 380.2 | 380.2 |
| Int-8 | 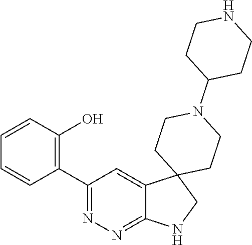 [SMILES: Oc1ccccc1-c1cc2c(nn1)NCC21CCN(C2CCNCC2)CC1] | 2-(1-(piperidin-4-yl)- 6′,7′-dihydrospiro [piperidine-4,5′- pyrrolo[2,3- c]pyridazin]-3′-yl) phenol | 366.2 | 366.3 |
| Int-9 | 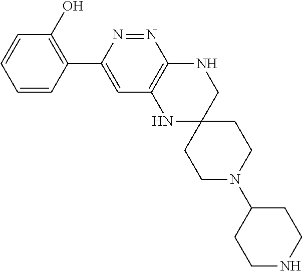 [SMILES: Oc1ccccc1-c1cc2c(nn1)NCC1(CCN(C3CCNCC3)CC1)N2] | 2-(1-(piperidine-4-yl)- 7′,8′-dihydro-5′H-spiro [piperidine-4,6′- pyrazino [2,3- c]pyridazin]-3′-yl) phenol | 381.2 | 381.3 |
| Int-10 | 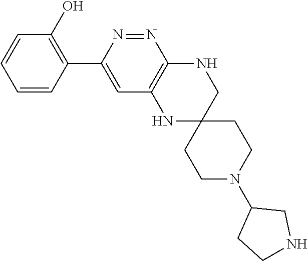 [SMILES: Oc1ccccc1-c1cc2c(nn1)NCC1(CCN(C3CCNC3)CC1)N2] | 2-(1-(pyrrolidin-3-yl)- 7′,8′-dihydro-5′H-spiro [piperidine-4,6′- pyrazino [2,3- c]pyridazin]-3′-yl) phenol | 367.2 | 367.3 |
| Int-11 | 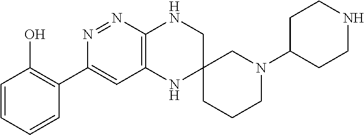 [SMILES: Oc1ccccc1-c1cc2c(nn1)NCC1(CCCN(C3CCNCC3)C1)N2] | 2-(1-(piperidine-4-yl)- 7′,8′-dihydro-5′H-spiro [piperidine-3,6′- pyrazino [2,3- c]pyridazin]-3′-yl) phenol | 381.2 | 381.2 |
| Int-12 | 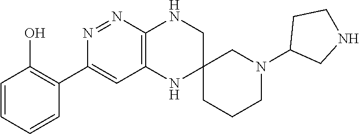 [SMILES: Oc1ccccc1-c1cc2c(nn1)NCC1(CCCN(C3CCNC3)C1)N2] | 2-(1-(pyrrolidin-3-yl)- 7′,8′-dihydro-5′H-spiro [piperidine-3,6′- pyrazino [2,3- c]pyridazin]-3′-yl) phenol | 367.2 | 367.2 |
| Int-13 | 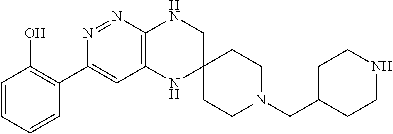 [SMILES: Oc1ccccc1-c1cc2c(nn1)NCC1(CCN(CC3CCNCC3)CC1)N2] | 2-(1-(piperidin-4- ylmethyl)-7′,8′-dihydro- 5′H-spiro[piperidine- 4,6′-pyrazino[2,3- c]pyridazin]-3′-yl) phenol | 395.2 | 395.2 |
| Int-14 | 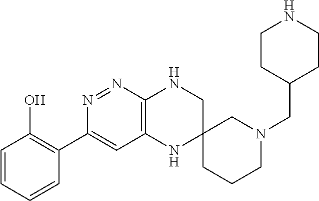 [SMILES: Oc1ccccc1-c1cc2c(nn1)NCC1(CCCN(CC3CCNCC3)C1)N2] | 2-(1-(piperidin-4- ylmethyl)-7′,8′-dihydro- 5′H-spiro[piperidine- 3,6′-pyrazino [2,3- c]pyridazin]-3′-yl) phenol | 395.2 | 395.3 |

### Example 1: (2S,4R)-4-hydroxy-1-((2R)-2-(3-(2-(3-(3′-(2-hydroxyphenyl)-6′,7′-dihydrospiro [piperidine-4,5′-pyrrolo[2,3-c]pyridazin]-1-yl)pyrrolidin-1-yl)ethoxy) isoxazol-5-yl)-3-methylbutanoyl)-N—((S)-1-(4-(4-methylthiazol-5-yl)phenyl)ethyl) pyrrolidine-2-carboxamide

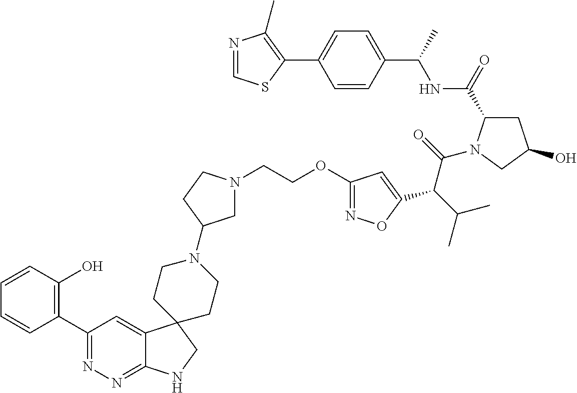 [SMILES: Cc1ncsc1-c1ccc([C@H](C)NC(=O)[C@@H]2C[C@@H](O)CN2C(=O)[C@@H](c2cc(OCCN3CCC(N4CCC5(CC4)CNc4nnc(-c6ccccc6O)cc45)C3)no2)C(C)C)cc1]

### Step 1: (2S,4R)-4-((tert-butyldimethylsilyl)oxy)-1-((2R)-2-(3-(2-(3-(3′-(2-hydroxyphenyl)-6′,7′-dihydrospiro[piperidine-4,5′-pyrrolo[2,3-c]pyridazin]-1-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide

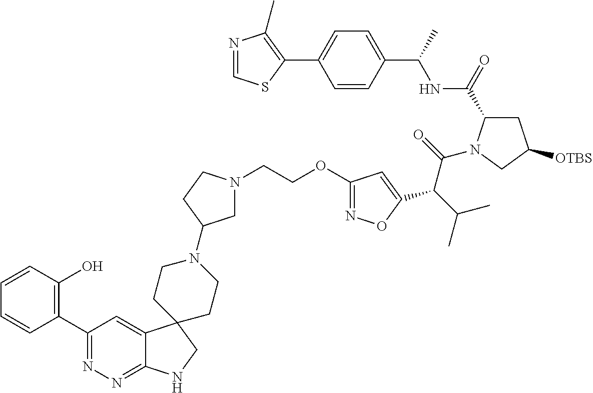 [SMILES: Cc1ncsc1-c1ccc([C@H](C)NC(=O)[C@@H]2C[C@@H](O[Si](C)(C)C(C)(C)C)CN2C(=O)[C@@H](c2cc(OCCN3CCC(N4CCC5(CC4)CNc4nnc(-c6ccccc6O)cc45)C3)no2)C(C)C)cc1]

A mixture of (2S,4R)-1-((R)-2-(3-(2-bromoethoxy)isoxazol-5-yl)-3-methylbutanoyl)-4-((tert-butyldimethylsilyl)oxy)-N—((S)-1-(4-(4-methylthiazol-5-yl)phenyl)ethyl)pyrrolidine-2-carboxamide (164 mg, 0.23 mmol) and 2-(1-(pyrrolidin-3-yl)-6′,7′-dihydrospiro[piperidine-4,5′-pyrrolo[2,3-c]pyridazin]-3′-yl)phenol (40 mg, 0.11 mmol) in DMF (5 mL) was added sodium bicarbonate (0.14 mL, 1.14 mmol). The resulted mixture was stirred at 60° C. for 16 h. The reaction mixture was diluted with H₂O and extracted with EA. The organic layers were combined, dried over Na₂SO₄, filtered, and concentrated. The residue was purified by column chromatography on a silica gel column to give the desired product as a yellow oil (20 mg, 0.02 mmol, 17.7% yield). LCMS m/z calcd for C₅₃H₇₂N₉O₆SSi [M+H]⁺: 990.5; found: 990.7.

### Step 2: (2S,4R)-4-hydroxy-1-((2R)-2-(3-(2-(3-(3′-(2-hydroxyphenyl)-6′,7′-dihydrospiro [piperidine-4,5′-pyrrolo[2,3-c]pyridazin]-1-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide

(2S,4R)-4-((tert-butyldimethylsilyl)oxy)-1-((2R)-2-(3-(2-(3-(3′-(2-hydroxyphenyl)-6′,7′-dihydrospiro[piperidine-4,5′-pyrrolo[2,3-c]pyridazin]-1-yl)pyrrolidin-1-yl)ethoxy) isoxazole-5-yl)-3-methylbutanoyl)-N—((S)-1-(4-(4-methylthiazol-5-yl)phenyl)ethyl)pyrrolidine-2-carboxamide (20 mg, 0.02 mmol) was added to TFA (3.0 mL). The resulted mixture was stirred at 40° C. for 1 h. The volatiles were removed and the residue was purified by prep-HPLC, eluted with CH₃CN in H₂O (0.1% NH₄OH) from 10% to 95% to give (2S,4R)-4-hydroxy-1-((2R)-2-(3-(2-(3-(3′-(2-hydroxyphenyl)-6′,7′-dihydrospiro[piperidine-4,5′-pyrrolo[2,3-c]pyridazin]-1-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide (1.9 mg, 0.002 mmol, 10.5% yield) as a white solid. LCMS m/z calcd for C₄₇H₅₈N₉O₆S [M+H]⁺: 876.4; found: 876.4. ¹H NMR (400 MHz, CD₃OD) δ 8.87 (s, 1H), 7.97-7.96 (m, 1H), 7.85-7.83 (m, 1H), 7.45-7.39 (m, 4H), 7.25-7.23 (m, 1H), 6.95-6.91 (m, 2H), 6.03 (s, 1H), 5.11-5.01 (m, 1H), 4.62-4.33 (m, 4H), 3.85-3.82 (m, 1H), 3.69-3.59 (m, 4H), 3.00-2.92 (m, 4H), 2.78-2.77 (m, 1H), 2.63-2.62 (m, 1H), 2.52-2.36 (m, 5H), 2.18-1.94 (m, 7H), 1.83-1.80 (m, 2H), 1.60-1.50 (m, 4H), 1.05-1.04 (m, 3H), 0.92-0.88 (m, 3H).

### Examples 2-8

Examples 2-8 shown below in Table 3 were prepared according to the same method as Example 1 using appropriate starting materials.

TABLE 3

| | | Calcd. | Found | |
|---|---|---|---|---|
| | | (M + H)⁺ | (M + H)⁺ | |
| Ex # | Name | m/z | m/z | ¹HNMR |
| Ex 2 | (2S,4R)-4-hydroxy-1-((R)-2-(3- | 904.4 | 904.6 | ¹HNMR (CD₃OD-d₄, 400 |
| | (2-(4-(3′-(2-hydroxyphenyl)- | | | MHz): δ 8.86 (s, 1H), 7.59(s, |
| | 7′,8′-dihydro-5′H-spiro | | | 1H), 7.45-7.39 (m, 4H), |
| | [piperidine-4,6′-pyrazino[2,3- | | | 7.21-7.22 (m, 1H), 7.09 (s, |
| | c]pyridazin]-1-yl)piperidin-1- | | | 1H), 6.89-6.87 (m, 2H), 6.01 |
| | yl)ethoxy)isoxazol-5-yl)-3- | | | (s, 1H), 5.03-5.00 (m, 2H), |
| | methylbutanoyl)-N-((S)-1-(4- | | | 4.57-4.32 (m, 4H), 3.82-3.62 |
| | (4-methylthiazol-5-yl)phenyl) | | | (m, 2H), 3.14-3.11 (m, 2H), |
| | ethyl)pyrrolidine-2- | | | 2.84-2.73 (m, 6H), 2.57-2.23 |
| | carboxamide | | | (m, 8H), 1.95-1.52(m, 10H), |
| | | | | 1.52-1.50 (m, 3H), 1.33- |
| | | | | 1.30(m, 2H), 1.05-1.04 (m, |
| | | | | 3H), 0.93-0.92 (m, 3H). |
| Ex 3 | (2S,4R)-4-hydroxy-1-((R)-2-(3- | 904.4 | 904.4 | |
| | (2-(4-((3′-(2-hydroxyphenyl)- | | | |
| | 6′,7′-dihydrospiro[piperidine- | | | |
| | 4,5′-pyrrolo[2,3-c]pyridazin]-1- | | | |
| | yl)methyl)piperidin-1- | | | |
| | yl)ethoxy)isoxazol-5-yl)-3- | | | |
| | methylbutanoyl)-N-((S)-1-(4- | | | |
| | (4-methylthiazol-5-yl)phenyl) | | | |
| | ethyl)pyrrolidine-2- | | | |
| | carboxamide | | | |
| Ex 4 | (2S,4R)-4-hydroxy-1-((2R)-2- | 891.4 | 891.3 | ¹HNMR(CD₃OD-d₄, 400 |
| | (3-(2-(3-(3′-(2-hydroxyphenyl)- | | | MHz): δ 7.50-7.40 (m, 7H), |
| | 7′,8′-dihydro-5′H-spiro | | | 7.26-7.24 (m, 1H), 7.05-7.00 |
| | [piperidine-3,6′-pyrazino[2,3- | | | (m, 2H), 6.09-6.04 (m, 1H), |
| | c]pyridazin]-1-yl)pyrrolidin-1- | | | 5.04-7.01 (m, 2H), 4.61-4.43 |
| | yl)ethoxy)isoxazol-5-yl)-3- | | | (m, 4H), 3.87-3.34 (m, 16H), |
| | methylbutanoyl)-N-((S)-1-(4- | | | 3.26-3.12 (m, 2H), 2.55-2.51 |
| | (4-methylthiazol-5-yl)phenyl) | | | (m, 5H), 2.38-2.16 (m, |
| | ethyl)pyrrolidine-2- | | | 6H), 1.60-1.50 (m, 3H), 1.06- |
| | carboxamide | | | 1.04 (m, 3H), 0.91-0.87 (m, |
| | | | | 3H) |
| Ex 5 | (2S,4R)-4-hydroxy-1-((2R)-2- | 905.4 | 905.4 | ¹HNMR(CD₃OD-d₄, 400 |
| | (3-(2-(4-(3′-(2-hydroxyphenyl)- | | | MHz): δ 7.50-7.40 (m, 7H), |
| | 7′,8′-dihydro-5′H-spiro | | | 7.26-7.24 (m, 1H), 7.05-7.00 |
| | [piperidine-3,6′-pyrazino[2,3- | | | (m, 2H), 6.09-6.04 (m, 1H), |
| | c]pyridazin]-1-yl)piperidin-1- | | | 5.04-7.01 (m, 2H), 4.61-4.43 |
| | yl)ethoxy)isoxazol-5-yl)-3- | | | (m, 4H), 3.87-3.34 (m, 16H), |
| | methylbutanoyl)-N-((S)-1-(4- | | | 3.26-3.12 (m, 2H), 2.55-2.51 |
| | (4-methylthiazol-5-yl)phenyl) | | | (m, 5H), 2.38-2.16 (m, |
| | ethyl)pyrrolidine-2- | | | 6H), 1.60-1.50 (m, 3H), 1.06- |
| | carboxamide | | | 1.04 (m, 3H), 0.91-0.87(m, |
| | | | | 3H) |
| Ex 6 | (2S,4R)-4-hydroxy-1-((2R)-2- | 891.4 | 891.4 | ¹HNMR (CD₃OD-d₄, 400 |
| | (3-(2-(3-(3′-(2-hydroxyphenyl)- | | | MHz): δ 8.86 (s, 1H), 7.59(s, |
| | 7′,8′-dihydro-5′H-spiro | | | 1H), 7.45-7.39 (m, 4H), |
| | [piperidine-4,6′-pyrazino[2,3- | | | 7.21-7.22 (m, 1H), 7.09 (s, |
| | c]pyridazin]-1-yl)pyrrolidin-1- | | | 1H), 6.89-6.87 (m, 2H), 6.01 |
| | yl)ethoxy)isoxazol-5-yl)-3- | | | (s, 1H), 5.03-5.00 (m, 2H), |
| | methylbutanoyl)-N-((S)-1-(4- | | | 4.57-4.32 (m, 4H), 3.82-3.62 |
| | (4-methylthiazol-5-yl)phenyl) | | | (m, 2H), 3.14-3.11 (m, 2H), |
| | ethyl)pyrrolidine-2- | | | 2.84-2.73 (m, 6H), 2.57-2.23 |
| | carboxamide | | | (m, 8H), 1.95-1.52(m, 10H), |
| | | | | 1.52-1.50 (m, 3H), 1.33- |
| | | | | 1.30(m, 2H), 1.05-1.04 (m, |
| | | | | 3H), 0.93-0.92 (m, 3H) |
| Ex 7 | (2S,4R)-4-hydroxy-1-((2R)-2- | 919.5 | 919.6 | ¹HNMR (CD₃OD-d₄, 400 |
| | (3-(2-(4-((3′-(2-hydroxyphenyl)- | | | MHz): δ 9.93 (s, 1H), 7.57- |
| | 7′,8′-dihydro-5′H-spiro | | | 7.42(m, 6H), 7.27-7.02 (m, |
| | [piperidine-3,6′-pyrazino[2,3- | | | 3H), 6.10 (s, 1H), 5.05-5.03 |
| | c]pyridazin]-1-yl)methyl) | | | (m, 2H), 4.61-4.44 (m, 4H), |
| | piperidin-1-yl)ethoxy)isoxazol- | | | 3.61-3.30 (m, 12H), 3.21 (m, |
| | 5-yl)-3-methylbutanoyl)-N- | | | 4H), 2.61 (s, 3H), 2.38-1.93 |
| | ((S)-1-(4-(4-methylthiazol-5- | | | (m, 10H), 1.57-1.51(m, 5H), |
| | yl)phenyl)ethyl) pyrrolidine-2- | | | 1.05-1.04 (m, 3H), 0.88-0.87 |
| | carboxamide | | | (m, 3H) |
| Ex 8 | (2S,4R)-4-hydroxy-1-((R)-2-(3- | 919.5 | 919.5 | |
| | (2-(4-((3′-(2-hydroxyphenyl)- | | | |
| | 7′,8′-dihydro-5′H-spiro | | | |
| | [piperidine-4,6′-pyrazino[2,3- | | | |
| | c]pyridazin]-1-yl)methyl) | | | |
| | piperidin-1-yl)ethoxy)isoxazol- | | | |
| | 5-yl)-3-methylbutanoyl)-N- | | | |
| | ((S)-1-(4-(4-methylthiazol-5- | | | |
| | yl)phenyl)ethyl) pyrrolidine-2- | | | |
| | carboxamide | | | |

### Example A—Biological Assays

SMARCA2 HiBiT and SMARCA4 HiBiT Degradation Assay (cellular)

### Preparation of SMARCA2/4-HiBiT Knock-In Cells

SMARCA2-HiBiT knock-in Hela monoclonal cell (CS302366) and SMARCA4-HiBiT knock-in Hela monoclonal cell (CS3023226) were purchased from Promega. The heterozygous HiBiT-knock-in was confirmed by sanger sequence in both SMARCA2-HiBiT and SMARCA4-HiBiT monoclonal cells.

### SMARCA2 HiBiT and SMARCA4 HiBiT Degradation Assay in HeLa Cells

Dispense 10 μL aliquot of prepared Hela-SMARCA2-HiBiT or Hela-SMARCA4-HiBiT cells (1:1 ratio of cells:Trypan Blue (#1450013, Bio-Rad)) onto cell counting slide (#145-0011, Bio-Rad) and obtain cell density and cell viability using cell counter (TC20, Bio-Rad). Remove appropriate volume of resuspended cells from culture flask to accommodate 2500 cells/well @ 20 μL/well. Transfer Hela-HiBiT cells to 50 ml conical (#430290, Corning). Spin down at 1000 rpm for 5 min using tabletop centrifuge (SPINCHRON 15, Beckman). Discard supernatant and resuspend cell pellet in modified EMEM (#30-2003, ATCC) cell culture media containing 10% FBS (F2422-500ML, Sigma), and 1× Penicillin/Streptomycin (200 g/l) (30-002-CI, Corning) to a cell density of 125,000 cells/ml. Dispense 20 μL of resuspended Hela-HiBit cells per well in 384-well TC treated plate (#12-565-343, Thermo Scientific) using standard cassette (#50950372, Thermo Scientific) on Multidrop Combi (#5840310, Thermo Scientific) inside laminar flow cabinet. Dispense test compounds onto plates using digital liquid dispenser (D300E, Tecan). Incubate plates in humidified tissue culture incubator @37° C. for 18 hours. Add 20 ul of prepared Nano-Glo® HiBiT Lytic detection buffer (N3050, Promega) to each well of 384-well plate using small tube cassette (#24073295, Thermo Scientific) on Multidrop Combi, incubate @ RT for 30-60 min. Read plates on microplate reader (Envision 2105, PerkinElmer) using 384 well Ultra-Sensitive luminescence mode. Raw data files and compound information reports are swept into centralized data lake and deconvoluted using automated scripts designed by TetraScience, Inc. Data analysis, curve-fitting and reporting done in Dotmatics Informatics Suite using Screening Ultra module.

The results are shown below in Table 4 for the HiBiT assay. In Table 4, the DC₅₀/Dₘₐₓ% (SM2-HeLa) refers to SMARCA2 degradation potency/maximum SMARCA2 degradation within the concentrations tested in HeLa HiBiT assay. In Table 4, the DC₅₀/Dₘₐₓ% (SM4-HeLa) refers to SMARCA4 degradation potency/maximum SMARCA4 degradation within the concentrations tested in HeLa HiBiT assay.

In Table 4, A=IC₅₀ or DC₅₀<0.1 μM; B=0.1 μM=<IC₅₀ or DC₅₀<1 μM; C=1 μM=<IC₅₀ or DC₅₀<10 μM; D=IC₅₀ or DC₅₀>=10 μM; or A=Dₘₐₓ>75%; B=50%<Dₘₐₓ<=75%; C=Dₘₐₓ<=50%.

TABLE 4

| Example | DC₅₀/Dₘₐₓ % (SM2-HeLa) | DC₅₀/Dₘₐₓ % (SM4-HeLa) |
|---|---|---|
| 1 | A/A | D/C |
| 2 | A/B | A/A |
| 3 | A/B | D/C |
| 4 | A/B | A/A |
| 5 | A/B | A/A |
| 6 | C/C | B/A |
| 7 | A/A | A/A |
| 8 | A/A | A/A |

1. A compound of Formula I
A′-L-B (I)
or a pharmaceutically acceptable salt or solvate thereof, wherein
A′ is a heterocycle that binds SMARCA2;
L is a bond or chemical moiety that links A′ and B;
B is a VHL binding moiety;
wherein A′ is defined as formula (II) 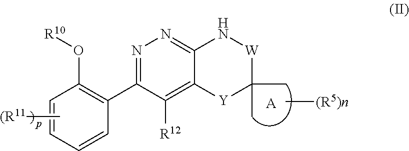
wherein
ring A is a carbocycle or a heterocycle;
W is a bond or (CR¹R²)ₘ;
Y is a bond, (CR¹R²)ₘ, —O—(CR¹R²)ₘ—, —S—(CR¹R²)ₘ—, —S(O)x-(CR¹R²)ₘ—, or —NR^{a}—(CR¹R²)ₘ—;
m is 0, 1, 2 or 3;
n is 0, 1, 2, 3, or 4;
R¹⁰ is H, —C(O)R^{f}, or —P(O)(OR^{g})₂; wherein R^{f} and R^{g} are independently H, C₁₋₄ alkyl, C₁₋₄ substituted alkyl, C₃₋₈ cyclcoalkyl, C₃₋₈ substituted cyclcoalkyl, C₃₋₈ heterocyclcoalkyl, or C₃₋₈ substituted heterocyclcoalkyl;
p is 1, 2, 3 or 4;
each R¹¹ is independently H, D, halogen, —OH, —OR¹², —CN, —NO₂, —C₁-C₆alkyl, —C₂-C₆alkenyl, —C₂-C₆alkynyl;
each R¹² is independently H, D, halogen, C₁-C₄ alkoxide, C₁-C₄ alkyl, haloalkyl or CN;
each R¹, R², or R⁵ is independently H, D, halogen, —C₁-C₆ alkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl, C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)OR^{c}OR^{b}, —S(O)₂R^{b}, —S(O)₂NR^{c}R^{d}, or SiR^{b}₃; or R¹ and R² taken together with the carbon atom they are attached to form —C═O.
each R^{a} and R^{b}, is independently H, D, —C₁-C₆ alkyl, —C₂-C₆ alkenyl, —C₂-C₆ alkynyl, C₀-C₁alk-aryl, cycloalkyl, cycloalkenyl, C₀-C₁alk-heteroaryl, heterocycloalkyl, or heterocycloalkenyl;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl;
or R^{c} and R^{d}, together with the atom to which they are both attached, form a monocyclic or multicyclic heterocycloalkyl, or a monocyclic or multicyclic heterocyclo-alkenyl group; and
wherein when L is a not a bond, L is a chemical moiety represented by the formula:
-(A)_{q}-,
q is an integer from 1 to 14
each A is independently selected from the group consisting of CR^{1a}R^{1b}, O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR(CR^{1a}R^{b})₁₋₄, NR^{1c}C(═NCN)NR^{1d}NR^{1c}C(═NCN), NR^{1c}C(═CNO₂)NR^{1d}, 3-11 membered cycloalkyl, optionally substituted with 0-6 R^{1a} and/or R^{1b} groups, 3-11 membered heteocyclyl optionally substituted with 0-6 R^{1a} and/or R^{1b} groups, aryl optionally substituted with 0-6 R^{1a} and/or R^{1b} groups, or heteroaryl optionally substituted with 0-6 R^{1a} and/or R^{1b} groups;
wherein R^{1a}, R^{1b}, R^{1c}, R^{1d} and R^{1e} are each independently, —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and where R^{1a} or R^{1b}, each independently may be optionally linked to other groups to form cycloalkyl and/or heterocyclyl moiety, optionally substituted with 0-4 R^{1e} groups;
or a pharmaceutically acceptable salt or solvate thereof.

2. (canceled)

3. (canceled)

4. The compound of claim 1 wherein W is a bond.

5. (canceled)

6. (canceled)

7. (canceled)

8. (canceled)

9. The compound of claim 1 wherein Y is a bond.

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. The compound of claim 1, that is a compound of formula III, formula IV, formula V, formula VI, or formula VII: 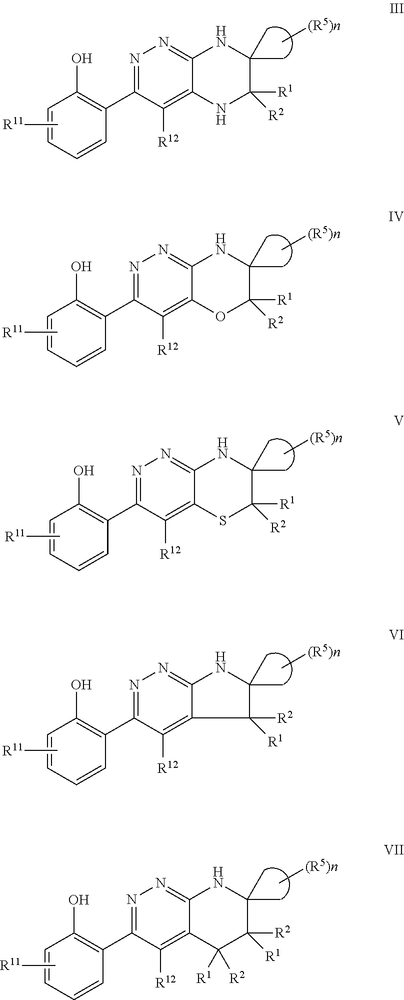
or a pharmaceutically acceptable salt or solvate thereof; wherein L is directly attached to ring A in any of the above formulae or wherein L is attached to ring A by an R⁵ group in any of the above formulae; or wherein L is attached to A′ by an R¹ or R² group in any of the above formulae.

26. The compound of claim 25, that is a compound of formula VII, formula IX, formula X, formula XI, or formula XII: 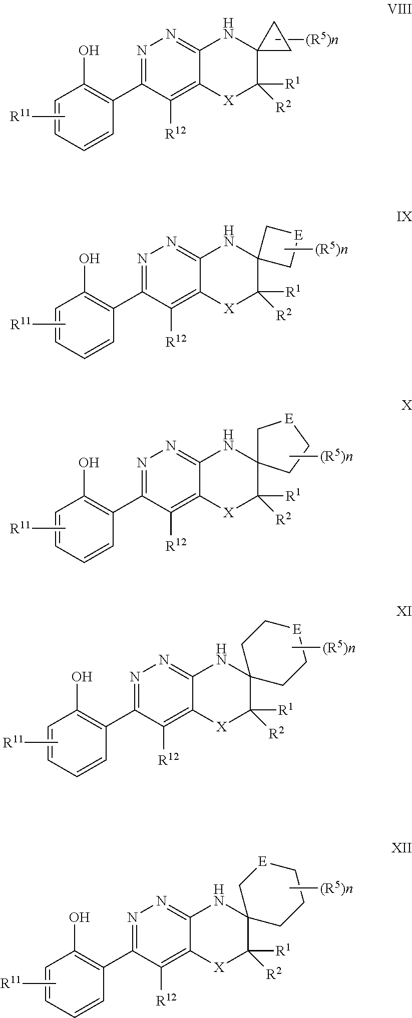
or a pharmaceutically acceptable salt or solvate thereof;
wherein E is CR¹R², O, C(O)NR^{c}R^{d}, N-L-B, C(R¹)(L-B), or NR^{b};
wherein when E is not N-L-B, C(R¹)(L-B), L is attached to ring A by an R¹, R², or R⁵ group included in E in any of the above formulae; or wherein L is attached to A′ by an R¹ or R² group in any of the above formulae; and wherein X is a bond, NR^{a}, O, S or CR¹R².

27. The compound of claim 1, that is a compound of formula XIII, formula XIV, formula XV, formula XVI, formula XVII, formula XVIII, formula XIX, formula XX, formula XXI, or formula XXII: 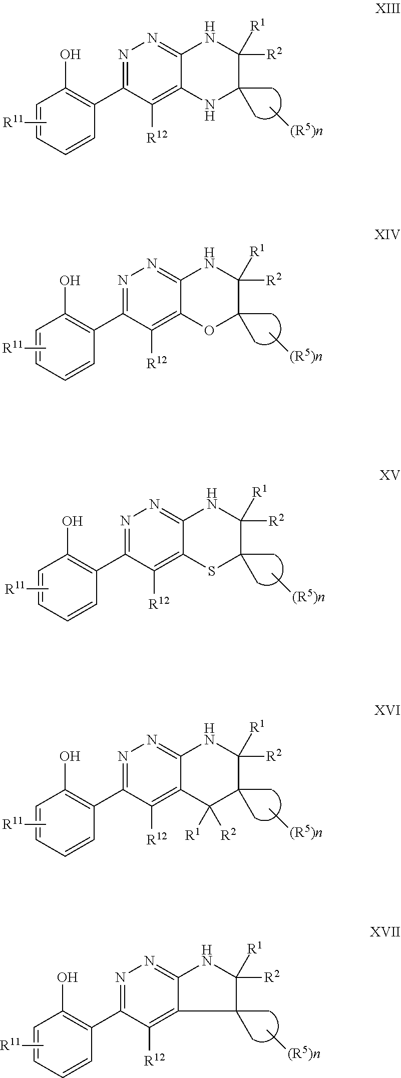 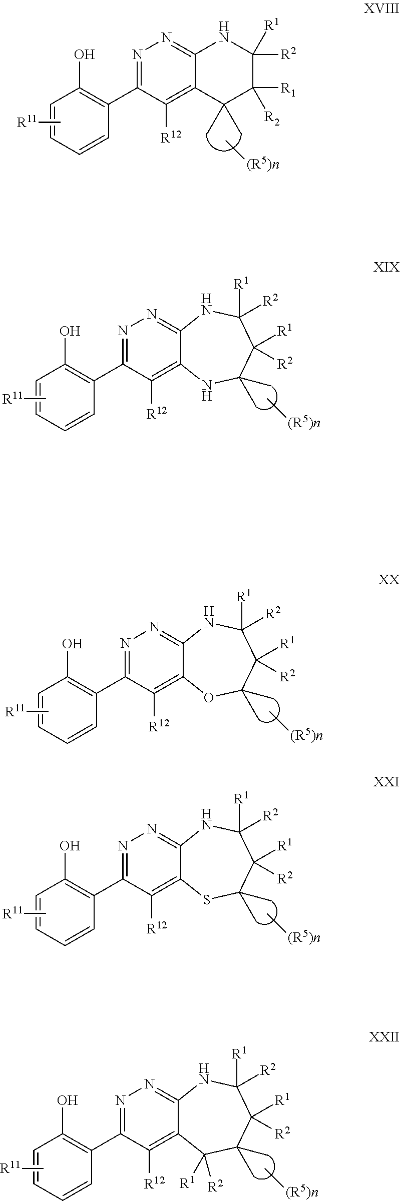
or a pharmaceutically acceptable salt or solvate thereof; wherein L is directly attached to ring A in any of the above formulae or wherein L is attached to ring A by an R⁵ group in any of the above formulae; or wherein L is attached to A′ by an R¹ or R² group in any of the above formulae.

28. The compound of claim 27, that is a compound of formula XXIII, formula XXIV, formula XXV, formula XXVI, or formula XXVII: 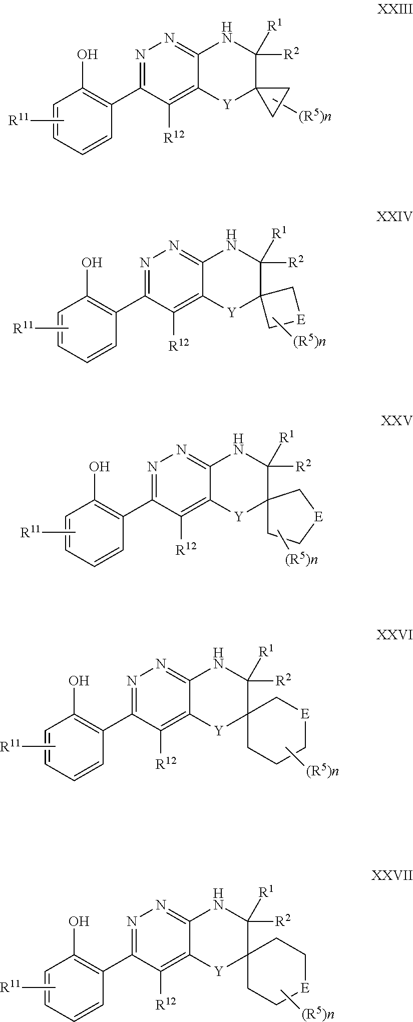
or a pharmaceutically acceptable salt or solvate thereof;
wherein E is CR¹R², O, C(O)NR^{c}R^{d}, -L-B, C(R¹)(L-B), or NR^{b}; wherein when E is not N-L-B or C(R¹)(L-B), L is attached to ring A by an R¹, R², or R⁵ group included in E in any of the above formulae′ or wherein L is attached to A′ by an R¹ or R² group in any of the above formulae;
and wherein Y is a bond, NR^{a}, O, S or CR¹R².

29. The compound of claim 1, that is a compound of formula XXVIII: 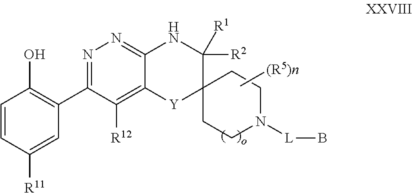
or a pharmaceutically acceptable salt or solvate thereof; wherein o is 0, 1 or 2 and wherein Y is a bond, NR^{a}, O, S or CR¹R².

30. The compound of claim 29, wherein Y is a bond.

31. (canceled)

32. (canceled)

33. (canceled)

34. (canceled)

35. The compound of claim 29, wherein R¹² is H.

36. (canceled)

37. (canceled)

38. (canceled)

39. The compound of claim 29, wherein R¹¹ is H.

40. (canceled)

41. (canceled)

42. The compound of claim 1, that is a compound of formula XXIX: 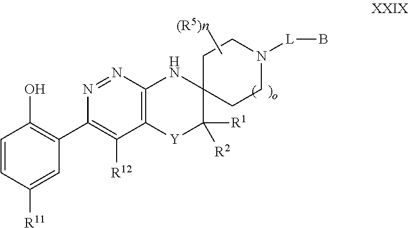
or a pharmaceutically acceptable salt or solvate thereof; wherein o is 0, 1 or 2 and wherein Y is a bond, NR^{a}, O, S or CR¹R².

43. The compound of claim 42, wherein Y is a bond.

44. (canceled)

45. (canceled)

46. (canceled)

47. (canceled)

48. The compound of claim 42, wherein R¹² is H.

49. (canceled)

50. (canceled)

51. (canceled)

52. The compound of claim 42, wherein R¹¹ is H.

53. (canceled)

54. (canceled)

55. The compound according to claim 1, wherein B is a moiety having the Formula B-I 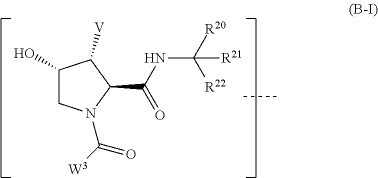
wherein
the dashed line ( [symbol] ) indicates the position of attachment of B-I to L;
V is H or F;
R²⁰ is optionally substituted phenyl, optionally substituted napthyl, or an optionally substituted 5-10 membered heteroaryl;
one of R²¹ or R²² is H, D, haloalkyl, optionally substituted alkyl, optionally substituted cycloalkyl, optionally substituted heterocycloalkyl, —COR^{g}, CONR^{h1}R^{h2}; the other of R²¹ or R²² is H or D;
or R²¹ and R²², together with the carbon atom to which they are both attached, form an optionally substituted 3-5 membered cycloalkyl, heterocyclyl;
W³ is an optionally substituted aryl, optionally substituted heteroaryl, or 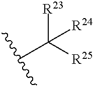
R²³ and R²⁴ are independently H, D, optionally substituted alkyl, optionally substituted cycloalkyl, or optionally substituted haloalkyl,
or R²³, R²⁴, and the carbon atom to which they are attached form an optionally substituted cycloalkyl or optionally substituted heterocyclyl;
R²⁵ is an optionally substituted heterocyclyl, optionally substituted heteroaryl, optionally substituted aryl, CONRⁱR^{j}, NRⁱR^{j}, 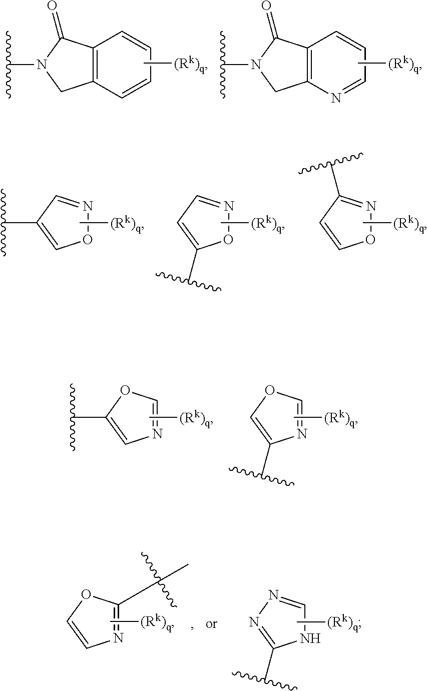
Rⁱ is selected from H or optionally substituted alkyl;
R^{j} is selected from H, —C(O)—* wherein * is a point of attachment to L, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (cycloalkyl)carbonyl, optionally substituted (heterocyclyl) carbonyl, or optionally substituted aralkyl;
each R^{k} is independently H, halo, optionally substituted alkoxy, cyano, optionally substituted alkyl, haloalkyl, or haloalkoxy;
each R^{g} is independently selected from H, optionally substituted alkyl or NR^{h1}R^{h2}; each R^{h1} and R^{h2} is independently H, D, optionally substituted alkyl,
or R^{h1} and R^{h2} together with the nitrogen atom to which they are attached form a 4-7 membered heterocyclyl; and
q is 0, 1, 2, 3, or 4.

56. (canceled)

57. (canceled)

58. (canceled)

59. (canceled)

60. (canceled)

61. (canceled)

62. (canceled)

63. (canceled)

64. The compound according to claim 55, wherein R²⁵ is

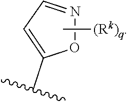

65. (canceled)

66. (canceled)

67. (canceled)

68. (canceled)

69. (canceled)

70. The compound according to claim 55, wherein R²⁰ is optionally substituted phenyl having the formula: 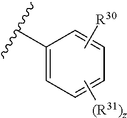
wherein
R³⁰ is H, D, halo, —CN, —OH, —NO₂, —NR^{h1}R^{h2}, —OR^{h1}, —CONR^{h1}R^{h2}, —NR^{h1}COR^{h2}, —SO₂NR^{h1}R^{h2}, —NR^{h1}SO₂R^{h2}, optionally substituted alkyl, optionally substituted alkoxyl, optionally substituted haloalkyl, optionally substituted haloalkoxy; optionally substituted aryl; optionally substituted heteroaryl; optionally substituted cycloalkyl; or optionally substituted heterocyclyl;
R³¹ is H, D, halo, CN, optionally substituted alkyl, optionally substituted haloalkyl, hydroxy, —NH(optionally substituted alkyl), —N(optionally substituted alkyl)₂, optionally substituted alkoxy, or optionally substituted haloalkoxy; and
z is 0, 1, 2, 3, or 4.

71. (canceled)

72. The compound according to claim 70, wherein R³⁰ is

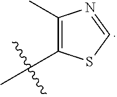 [SMILES: *c1scnc1C]

73. (canceled)

74. (canceled)

75. (canceled)

76. (canceled)

77. The compound according to claim 55, wherein B-I is a compound of formula: 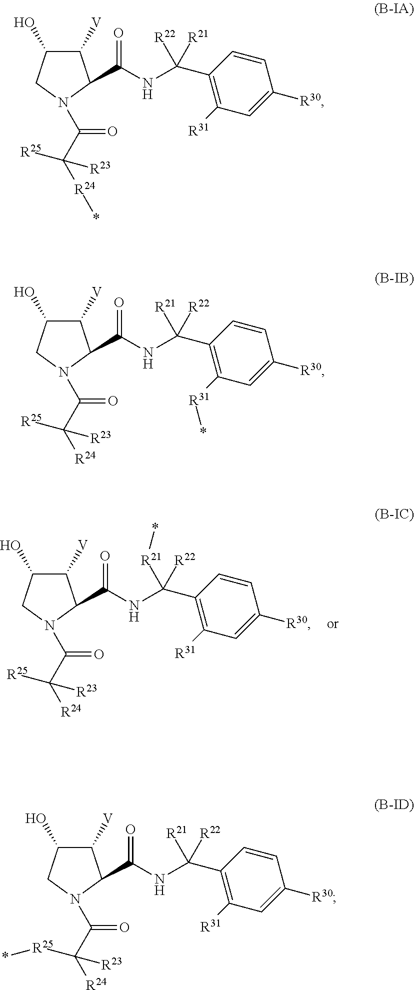
wherein * is a point of attachment to L.

78. (canceled)

79. The compound according to claim 77, wherein the compound of Formula I is a compound of Formula IA-7 or IA-8:

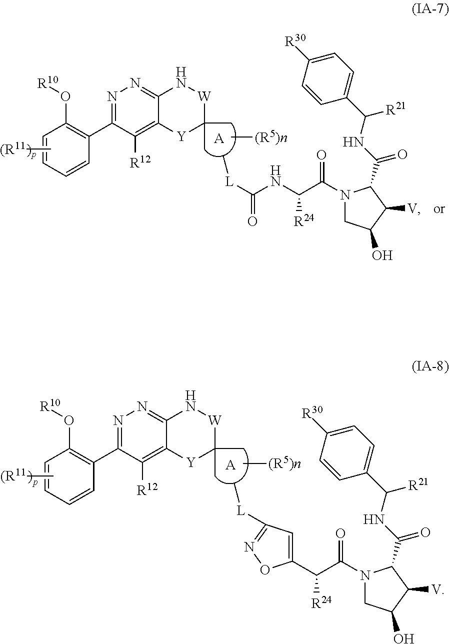

80. The compound according to claim 79, wherein the compound of Formula I is a compound of Formula IA-9, IA-10, IA-11 or IA-12:

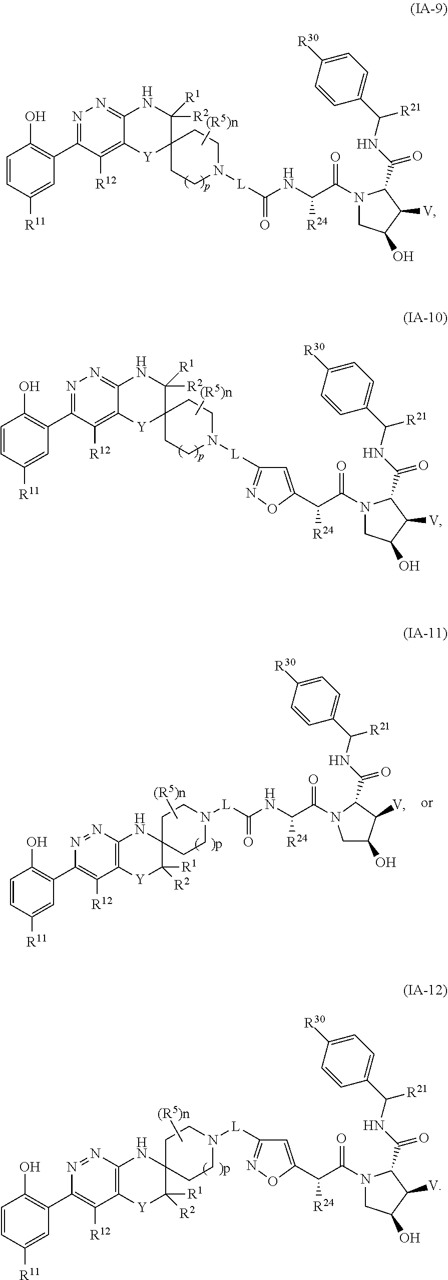

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. The compound according to claim 1, wherein L is —(CR^{1a}R¹)₁₋₅-(3-11 membered cycloalkyl optionally substituted with 0-6 R^{1a} and/or R^{1b} groups)-(CR^{1a}R^{1b})₁₋₅-A- wherein A is O, S, or NR^{1c}.

93. The compound according to claim 1, wherein L is —(CR^{1a}R^{b})₁₋₅-(3-11 membered heterocyclyl optionally substituted with 0-6 R^{1a} and/or R^{1b} groups)-(CR^{1a}R^{1b})₁₋₅-A- wherein A is O, S, or NR^{1c}.

94. (canceled)

95. (canceled)

96. (canceled)

97. (canceled)

98. The compound of claim 1, wherein the compound is:
(2S,4R)-4-hydroxy-1-((2R)-2-(3-(2-(3-(3′-(2-hydroxyphenyl)-6′,7′-dihydrospiro[piperidine-4,5′-pyrrolo[2,3-c]pyridazin]-1-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-(4-(3′-(2-hydroxyphenyl)-7′,8′-dihydro-5′H-spiro[piperidine-4,6′-pyrazino[2,3-c]pyridazin]-1-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-(4-((3′-(2-hydroxyphenyl)-6′,7′-dihydrospiro [piperidine-4,5′-pyrrolo[2,3-c]pyridazin]-1-yl)methyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((2R)-2-(3-(2-(3-(3′-(2-hydroxyphenyl)-7′,8′-dihydro-5′H-spiro[piperidine-3,6′-pyrazino[2,3-c]pyridazin]-1-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((2R)-2-(3-(2-(4-(3′-(2-hydroxyphenyl)-7′,8′-dihydro-5′H-spiro[piperidine-3,6′-pyrazino[2,3-c]pyridazin]-1-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((2R)-2-(3-(2-(3-(3′-(2-hydroxyphenyl)-7′,8′-dihydro-5′H-spiro[piperidine-4,6′-pyrazino[2,3-c]pyridazin]-1-yl)pyrrolidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((2R)-2-(3-(2-(4-((3′-(2-hydroxyphenyl)-7′,8′-dihydro-5′H-spiro[piperidine-3,6′-pyrazino[2,3-c]pyridazin]-1-yl)methyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
(2S,4R)-4-hydroxy-1-((R)-2-(3-(2-(4-((3′-(2-hydroxyphenyl)-7′,8′-dihydro-5′H-spiro[piperidine-4,6′-pyrazino[2,3-c]pyridazin]-1-yl)methyl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide;
or a pharmaceutically acceptable salt thereof.

99. The compound of claim 1 in the form of a pharmaceutically acceptable salt.

100. A pharmaceutical composition comprising a compound according to claim 1 and a pharmaceutically acceptable excipient.

101. A method of treating cancer in a subject in need thereof comprising administering to the subject a compound of claim 1 or a pharmaceutical composition comprising the compound.

102. The method of claim 101, wherein the cancer is SMARCA4 deleted cancer.

103. The method of claim 101 wherein the cancer is squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present disclosure include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.
